SEQUENCE LISTING (22 sequences)

SEQ ID NO: 1 - protein, 317 aa, Homo sapiens [sequence omitted]

SEQ ID NO: 2 - protein, 300 aa, Homo sapiens
AKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVRGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 3 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQTAQARLGADMEDVCGRLVQYRGEVQAMLGQSTEELRVRLASHLRKLRKRLLRDPDDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 4 - protein, 300 aa, Homo sapiens
AKVEQAVETEPEPELRQQTEWQSGQRWELALGRFWDYLRWVQTLSEQVQEELLSSQVTQELRALMDETMKELKAYKSELEEQLTPVAEETRARLSKELQTAQARLGADMEDVCGRLVQYRGEVQAMLGQSTEELRVRLASHLRKLRKRLLRDPDDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 5 - protein, 147 aa, Homo sapiens
MASHRLLLLCLAGLVFVSEAGPTGTGESKCPLMVKVLDAVRGSPAINVAVHVFRKAADDTWEPFASGKTSESGELHGLTTEEEFVEGIYKVEIDTKSYWKALGISPFHEHAEVVFTANDSGPRRYTIAALLSPYSYSTTAVVTNPKE

SEQ ID NO: 6 - protein, 1663 aa, Homo sapiens
MGPTSGPSLLLLLLTHLPLALGSPMYSIITPNILRLESEETMVLEAHDAQGDVPVTVTVHDFPGKKLVLSSEKTVLTPATNHMGNVTFTIPANREFKSEKGRNKFVTVQATFGTQVVEKVVLVSLQSGYLFIQTDKTIYTPGSTVLYRIFTVNHKLLPVGRTVMVNIENPEGIPVKQDSLSSQNQLGVLPLSWDIPELVNMGQWKIRAYYENSPQQVFSTEFEVKEYVLPSFEVIVEPTEKFYYIYNEKGLEVTITARFLYGKKVEGTAFVIFGIQDGEQRISLPESLKRIPIEDGSGEVVLSRKVLLDGVQNPRAEDLVGKSLYVSATVILHSGSDMVQAERSGIPIVTSPYQIHFTKTPKYFKPGMPFDLMVFVTNPDGSPAYRVPVAVQGEDTVQSLTQGDGVAKLSINTHPSQKPLSITVRTKKQELSEAEQATRTMQALPYSTVGNSNNYLHLSVLRTELRPGETLNVNFLLRMDRAHEAKIRYYTYLIMNKGRLLKAGRQVREPGQDLVVLPLSITTDFIPSFRLVAYYTLIGASGQREVVADSVWVDVKDSCVGSLVVKSGQSEDRQPVPGQQMTLKIEGDHGARVVLVAVDKGVFVLNKKNKLTQSKIWDVVEKADIGCTPGSGKDYAGVFSDAGLTFTSSSGQQTAQRAELQCPQPAARRRRSVQLTEKRMDKVGKYPKELRKCCEDGMRENPMRFSCQRRTRFISLGEACKKVFLDCCNYITELRRQHARASHLGLARSNLDEDIIAEENIVSRSEFPESWLWNVEDLKEPPKNGISTKLMNIFLKDSITTWEILAVSMSDKKGICVADPFEVTVMQDFFIDLRLPYSVVRNEQVEIRAVLYNYRQNQELKVRVELLHNPAFCSLATTKRRHQQTVTIPPKSSLSVPYVIVPLKTGLQEVEVKAAVYHHFISDGVRKSLKVVPEGIRMNKTVAVRTLDPERLGREGVQKEDIPPADLSDQVPDTESETRILLQGTPVAQMTEDAVDAERLKHLIVTPSGCGEQNMIGMTPTVIAVHYLDETEQWEKFGLEKRQGALELIKKGYTQQLAFRQPSSAFAAFVKRAPSTWLTAYVVKVFSLAVNLIAIDSQVLCGAVKWLILEKQKPDGVFQEDAPVIHQEMIGGLRNNNEKDMALTAFVLISLQEAKDICEEQVNSLPGSITKAGDFLEANYMNLQRSYTVAIAGYALAQMGRLKGPLLNKFLTTAKDKNRWEDPGKQLYNVEATSYALLALLQLKDFDFVPPVVRWLNEQRYYGGGYGSTQATFMVFQALAQYQKDAPDHQELNLDVSLQLPSRSSKITHRIHWESASLLRSEETKENEGFTVTAEGKGQGTLSVVTMYHAKAKDQLTCNKFDLKVTIKPAPETEKRPQDAKNTMILEICTRYRGDQDATMSILDISMMTGFAPDTDDLKQLANGVDRYISKYELDKAFSDRNTLIIYLDKVSHSEDDCLAFKVHQYFNVELIQPGAVKVYAYYNLEESCTRFYHPEKEDGKLNKLCRDELCRCAEENCFIQKSDDKVTLEERLDKACEPGVDYVYKTRLVKVQLSNDFDEYIMAIEQTIKSGSDEVQVGQQRTFISPIKCREALKLEEKKHYLMWGLSSDFWGEKPNLSYIIGKDTWVEHWPEEDECQDEENQKQCQDLGAFTESMVVFGCPN

SEQ ID NO: 7 - protein, 203 aa, Homo sapiens
SNLDEDIIAEENIVSRSEFPESWLWNVEDLKEPPKNGISTKLMNIFLKDSITTWEILAVSMSDKKGICVADPFEVTVMQDFFIDLRLPYSVVRNEQVEIRAVLYNYRQNQELKVRVELLHNPAFCSLATTKRRHQQTVTIPPKSSLSVPYVIVPLKTGLQEVEVKAAVYHHFISDGVRKSLKVVPEGIRMNKTVAVRTLDPER

SEQ ID NO: 8 - protein, 203 aa, Homo sapiens; identical to SEQ ID NO: 7

SEQ ID NO: 9 - protein, 1256 aa, Homo sapiens
MGPTSGPSLLLLLLTHLPLALGSPMYSIITPNILRLESEETVVLEAHDAQGDVPVTVIVHDFPGKKLVLSSEKTVLTPATNHMGNVTFMIPANREFKSEKGRNKFVTVQATFGAQVVEKVVLVSLQSGYLFIQTDKTIYTPGSTVLYRIFTVNHKLLPVGRTVMVNIEVPARGGPRGSRGTGLGEAKRSRETEKDTPEGVQFLYGKKVEGTAFVIFGIQDGEQRISLPESLKRIPIEDGLGEVVLSRKVLLEGVHNPRAEDLVGKSLYVSVTVILHSGSDMVQAERSGIPIVTSPYQIHFTKTPKYFKPGMPFDLMVFVTNPDGSPAYRVPVAVQGEDTVQSLTQGDGVAKLSINTHPSQKPLSITVRTKKQELSEAEQATSTMQALPYSTVGNSNNYLHLSVPRTELRPGETLNVNFLLRMDRAHEAKIRYYTYLIMNKGRLLKAGRQVREPGQDLVVLPLSITTDFIPSFRLVAYYTLIGASGQREVVADSVWVDVKDSCVGSLAGQSGQSEDRQPVPGQQMTLKIEGDHGARVVLVAVDKGVFVLNKKNKLTQSKIWDVVEKADIGCTPGSGKDYAGVFSDAGLTFTSSSGQQTAQRAELQCPQPAARRRRSVLLTEKRMDKVGKYPKELRKCCEDGMRENPMRFSCQRRTRFISLGEACKKVFLDCCNYITELRRQHARAGHLGLGRSDLDEDIIAEENIVSRSEFPESWLWNVEDLKEPPKNGISTKLMNIFLKDSITTWEILAVSMSDKKGERGCWLVPGRESASHIRQTRVSGSGGRGSGGARGLVACCTHTCPDPFSPWQVRVELLHNPAFCSLATTKRRHQQTVTIPPKSSLSVPYVIVPLKTGLQEVEVKAAVYHHFISDGVRKSLKVVPEGIRMNKTVAVRTLDPERLGREGVQKEDIPPADLSDQVPDTESETRILLQGTPVAQMTEDAVDAERLKHLIVTPSGCGEQNMIGMTPTVIAVHYLDETEQWEKFGLEKRQGALELIKKGYTQQLAFRQPSSAFAAFVKRAPSTWLTAYVVKVFSLAVNLIAIDSQVLCGAVKWLILEKQKPDGVFQEDAPVIHQEMIGGLRNNNEKDMALTAFVLISLQEAKDICEEQVNSLPGSITKAGDFLEANYMNLQRSYTVAIAGYALAQMGRLKGPLLNKFLTTAKDKNRWEDPGKQLYNVEATSYALLALLQLKDFDFVPPVVRWLNEQRYYGGGYGSTQASGPTAPRHMHPCLLRLPTGLLEKTLRPSEAVLHSHEPV

SEQ ID NO: 10 - protein, 349 aa, Homo sapiens
EGVQKEDIPPADLSDQVPDTESETRILLQGTPVAQMTEDAVDAERLKHLIVTPSGCGEQNMIGMTPTVIAVHYLDETEQWEKFGLEKRQGALELIKKGYTQQLAFRQPSSAFAAFVKRAPSTWLTAYVVKVFSLAVNLIAIDSQVLCGAVKWLILEKQKPDGVFQEDAPVIHQEMIGGLRNNNEKDMALTAFVLISLQEAKDICEEQVNSLPGSITKAGDFLEANYMNLQRSYTVAIAGYALAQMGRLKGPLLNKFLTTAKDKNRWEDPGKQLYNVEATSYALLALLQLKDFDFVPPVVRWLNEQRYYGGGYGSTQATFMVFQALAQYQKDAPDHQELNLDVSLQLPSR

SEQ ID NO: 11 - protein, 355 aa, Homo sapiens
EGVQKEDIPPADLSDQVPDTESETRILLQGTPVAQMTEDAVDAERLKHLIVTPSGCGEQNMIGMTPTVIAVHYLDETEQWEKFGLEKRQGALELIKKGYTQQLAFRQPSSAFAAFVKRAPSTWLTAYVVKVFSLAVNLIAIDSQVLCGAVKWLILEKQKPDGVFQEDAPVIHQEMIGGLRNNNEKDMALTAFVLISLQEAKDICEEQVNSLPGSITKAGDFLEANYMNLQRSYTVAIAGYALAQMGRLKGPLLNKFLTTAKDKNRWEDPGKQLYNVEATSYALLALLQLKDFDFVPPVVRWLNEQRYYGGGYGSTQASGPTAPRHMHPCLLRLPTGLLEKTLRPSEAVLHSHEPV

SEQ ID NO: 12 - protein, 505 aa, Homo sapiens
KIVLDPSGSMNIYLVLDGSDSIGASNFTGAKKCLVNLIEKVASYGVKPRYGLVTYATYPKIWVKVSEADSSNADWVTKQLNEINYEDHKLKSGTNTKKALQAVYSMMSWPDDVPPEGWNRTRHVIILMTDGLHNMGGDPITVIDEIRDLLYIGKDRKNPREDYLDVYVFGVGPLVNQVNINALASKKDNEQHVFKVKDMENLEDVFYQMIDESQSLSLCGMVWEHRKGTDYHKQPWQAKISVIRPSKGHESCMGAVVSEYFVLTAAHCFTVDDKEHSIKVSVGGEKRDLEIEVVLFHPNYNINGKKEAGIPEFYDYDVALIKLKNKLKYGQTIRPICLPCTEGTTRALRLPPTTTCQQQKEELLPAQDIKALFVSEEEKKLTRKEVYIKNGDKKGSCERDAQYAPGYDKVKDISEVVTPRFLCTGGVSPYADPNTCRGDSGGPLIVHKRSRFIQVGVISWGVVDVCKNQKRQKQVPAHARDFHINLFQVLPWLKEKLQDEDLGFL

SEQ ID NO: 13 - protein, 1231 aa, Homo sapiens
MRLLAKIICLMLWAICVAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNHGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR

SEQ ID NO: 14 - protein, 449 aa, Homo sapiens
MRLLAKIICLMLWAICVAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNHGRKFVQGKSIDVACHPGYALPKAQTTVTCMENGWSPTPRCIRVSFTL

SEQ ID NO: 15 - protein, 342 aa, Homo sapiens
MRLLAKIICLMLWAICVAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRR

SEQ ID NO: 16 - protein, 930 aa, Homo sapiens
MKPPRPVRTCSKVLVLLSLLAIHQTTTAEKNGIDIYSLTVDSRVSSRFAHTVVTSRVVNRANTVQEATFQMELPKKAFITNFSMNIDGMTYPGIIKEKAEAQAQYSAAVAKGKNAGLVKATGRNMEQFQVSVSVAPNAKITFELVYEELLKRRLGVYELLLKVRPQQLVKHLQMDIHIFEPQGISFLETESTFMTNQLVDALTTWQNKTKAHIRFKPTLSQQQKSPEQQETVLDGNLIIRYDVDRAISGGSIQIENGYFVHYFAPEGLTTMPKNVVFVIDKSGSMSGRKIQQTREALIKILDDLSPRDQFNLIVFSTEATQWRPSLVPASAENVNKARSFAAGIQALGGTNINDAMLMAVQLLDSSNQEERLPEGSVSLIILLTDGDPTVGETNPRSIQNNVREAVSGRYSLFCLGFGFDVSYAFLEKLALDNGGLARRIHEDSDSALQLQDFYQEVANPLLTAVTFEYPSNAVEEVTQNNFRLLFKGSEMVVAGKLQDRGPDVLTATVSGKLPTQNITFQTESSVAEQEAEFQSPKYIFHNFMERLWAYLTIQQLLEQTVSASDADQQALRNQALNLSLAYSFVTPLTSMVVTKPDDQEQSQVAEKPMEGESRNRNVHSGSTFFKYYLQGAKIPKPEASFSPRRGWNRQAGAAGSRMNFRPGVLSSRQLGLPGPPDVPDHAAYHPFRRLAILPASAPPATSNPDPAVSRVMNMKIEETTMTTQTPAPIQAPSAILPLPGQSVERLCVDPRHRQGPVNLLSDPEQGVEVTGQYEREKAGFSWIEVTFKNPLVWVHASPEHVVVTRNRRSSAYKWKETLFSVMPGLKMTMDKTGLLLLSDPDKVTIGLLFWDGRGEGLRLLLRDTDRFSSHVGGTLGQFYQEVLWGSPAASDDGRRTLRVQGNDHSATRERRLDYQEGPPGVEISCWSVEL

SEQ ID NO: 17 - protein, 242 aa, Homo sapiens
RLAILPASAPPATSNPDPAVSRVMNMKIEETTMTTQTPAPIQAPSAILPLPGQSVERLCVDPRHRQGPVNLLSDPEQGVEVTGQYEREKAGFSWIEVTFKNPLVWVHASPEHVVVTRNRRSSAYKWKETLFSVMPGLKMTMDKTGLLLLSDPDKVTIGLLFWDGRGEGLRLLLRDTDRFSSHVGGTLGQFYQEVLWGSPAASDDGRRTLRVQGNDHSATRERRLDYQEGPPGVEISCWSVEL

SEQ ID NO: 18 - protein, 256 aa, Homo sapiens
RLAILPASAPPATSNPDPAVSRVMNMKIEETTMTTQTPACPSCSRSRAPAVPAPIQAPSAILPLPGQSVERLCVDPRHRQGPVNLLSDPEQGVEVTGQYEREKAGFSWIEVTFKNPLVWVHASPEHVVVTRNRRSSAYKWKETLFSVMPGLKMTMDKTGLLLLSDPDKVTIGLLFWDGRGEGLRLLLRDTDRFSSHVGGTLGQFYQEVLWGSPAASDDGRRTLRVQGNDHSATRERRLDYQEGPPGVEISCWSVEL

SEQ ID NO: 19 - protein, 281 aa, Homo sapiens [sequence omitted]

SEQ ID NO: 20 - protein, 583 aa, Homo sapiens
MKLLHVFLLFLCFHLRFCKVTYTSQEDLVEKKCLAKKYTHLSCDKVFCQPWQRCIEGTCVCKLPYQCPKNGTAVCATNRRSFPTYCQQKSLECLHPGTKFLNNGTCTAEGKFSVSLKHGNTDSEGIVEVKLVDQDKTMFICKSSWSMREANVACLDLGFQQGADTQRRFKLSDLSINSTECLHVHCRGLETSLAECTFTKRRTMGYQDFADVVCYTQKADSPMDDFFQCVNGKYISQMKACDGINDCGDQSDELCCKACQGKGFHCKSGVCIPSQYQCNGEVDCITGEDEVGCAGFASVAQEETEILTADMDAERRRIKSLLPKLSCGVKNRMHIRRKRIVGGKRAQLGDLPWQVAIKDASGITCGGIYIGGCWILTAAHCLRASKTHRYQIWTTVVDWIHPDLKRIVIEYVDRIIFHENYNAGTYQNDIALIEMKKDGNKKDCELPRSIPACVPWSPYLFQPNDTCIVSGWGREKDNERVFSLQWGEVKLISNCSKFYGNRFYEKEMECAGTYDGSIDACKGDSGGPLVCMDANNVTYVWGVVSWGENCGKPEFPGFYTKVANYFDWISYHVGRPFISQYNV

SEQ ID NO: 21 - protein, 215 aa, Homo sapiens
GIVLTQSPGTLSLSSGEGATLSCRASQSVSNGQLTWYQQKPGQAPRLLIYGASSRATGIPDRFSGSGSGTDFTLTISRLEPEDFAVYYCHQYDGSPETFGQGTKVEINRTVAAPSVFIFPPSDEQLKSGTASVVCLLNNFYPREAKVQWKVDNALQSGNSQESVTEQDSKDSTYSLSSTLTLSKADYEKHKVYACEVTHQGLRSPVTKSFNRGEC

SEQ ID NO: 22 - protein, 396 aa, Homo sapiens [sequence omitted]

(19) United States
(12) Patent Application Publication
(10) Pub. No.: US 2010/0167937 A1
(43) Pub. Date: Jul. 1, 2010

(54) Multiple forms of Alzheimer's disease based on differences in concentrations of protein biomarkers in blood serum

(75) Inventors: Ira L. Goldknopf, The Woodlands, TX (US); Jennifer K. Bryson, The Woodlands, TX (US); Essam A. Sheta, The Woodlands, TX (US); Jaffer K. Khalil, The Woodlands, TX (US); Silvia C. Quintero, The Woodlands, TX (US)

Correspondence Address: BENJAMIN A. ADLER, PH.D., J.D., 8011 CANDLE LANE, HOUSTON, TX, 77071, US

(73) Assignee: Power3 Medical Products, Inc., The Woodlands, TX (US)

(21) Appl. No.: 12/217,885

(22) Filed: Jul. 8, 2008

(51) Int. Cl. C40B 30/00; G01N 33/68; C12Q 1/02; G01N 33/92; G01N 33/53; G01N 33/561

(52) U.S. Cl. 506/7; 436/86; 435/29; 436/71; 435/7.92; 204/461

(57) ABSTRACT

The present invention relates to identification and uses of biomarkers for neurodegenerative disease, including Alzheimer's disease, and the related diseases. More specifically, the present invention relates to the identification of protein biomarkers useful for the screening, diagnosis, and differentiation of Alzheimer's disease from Parkinson's disease, other neurodegenerative diseases, and normal controls, and in the monitoring of Alzheimer's disease severity and disease mechanisms in patients.

### CROSS REFERENCE TO RELATED APPLICATIONS

This application claims priority to U.S. Utility Patent Application Ser. No. 11/503,881 filed Aug. 14, 2006 which claims priority to U.S. Provisional patent application Ser. No. 60/708,992 filed on Aug. 17, 2005, now abandoned.

### BACKGROUND OF THE INVENTION

1. Field of the Invention

This invention relates to the identification of the relationships between two or more biomarkers for differential diagnosis of neurodegenerative disease. More specifically, the present invention relates to protein biomarkers for Alzheimer's disease, whereby lack of detection, and/or the quantity of a first protein biomarker in a biological sample from Alzheimer's disease patients correlates with significant differences in the quantities of other protein biomarkers of Alzheimer's disease. When Alzheimer's disease patients and age-matched normal control subjects are each placed into separate categories based on whether they do or do not have detectable quantities of the first protein biomarker, the protein identities of, and the differences in the quantities of the first protein biomarker and/or one or more other protein biomarkers in the biological sample provide opportunities: improve sensitivity and specificity of differential diagnosis; measure disease severity and monitor drug response; monitor drug clinical trial stratification of patients; indicate differences in neuronal degeneration mechanisms in the patients; measure the activity of these mechanisms of neuronal degeneration; determine which of these mechanisms of neuronal degeneration predominates; determine which biomarkers and disease mechanisms measure the severity of Alzheimer's disease in the patients; discover new targets for drug therapies; and develop companion diagnostics.

More particularly, the present invention relates to the identification of the relationships between two or more of an Apolipoprotein E4 protein, an Apolipoprotein E3 protein, an Apolipoprotein A-IV Protein, a Transthyretin protein, a Complement C3c1 protein, a Complement C3c2a protein, a complement C3dg protein, a Complement Factor Bb protein, a Complement Factor H/Hs protein, a Complement Factor I protein, an Immunoglobulin protein, a Haptoglobin protein, and/or an Inter-alpha Trypsin Inhibitor heavy chain (H4) related protein, as biomarkers for distinguishing between different categories or types of Alzheimer's disease, and for early detection, screening, diagnosis, differential diagnosis, and monitoring of disease severity and disease mechanisms of patients with Alzheimer's disease (AD), Alzheimer's disease Like (AD-Like) dementias, Amyotrophic Lateral Sclerosis (ALS, Lou Gehrig's disease), and Parkinson's disease.

2. Description of the Related Art

Proteomics is a new field of medical research wherein the proteins of an organism, including a human being are studied as a group, are identified, and linked to biological functions, including roles in a variety of disease states. With the completion of the mapping of the human genome, the identification of unique gene products, or proteins, has increased exponentially. In addition, molecular diagnostic testing for the presence of certain proteins already known to be involved in certain biological functions has progressed from research applications alone to use in disease screening and diagnosis for clinicians. However, proteomic testing for diagnostic purposes remains in its infancy. There is, however, a great deal of interest in using proteomics for the elucidation of potential disease biomarkers and their uses in diagnosis and treatment of diseases.

Detection of abnormalities in the genome, including genetic mutations and minor genetic variants, can reveal the risk or potential risk for individuals to develop a disease. The transition from such risk to the emergence of disease can be characterized as an expression of genomic abnormalities or other abnormalities, not of genetic origin, in the proteome, i.e. in proteins. Thus, the appearance of abnormalities in the proteome signals the beginning of the process of cascading effects that can result in the deterioration of the health of the patient. Therefore, detection of proteomic abnormalities at an early stage is desirable in order to allow for detection of disease either before it is established or in its earliest stages where treatment may be most effective.

Recent progress using a novel form of mass spectrometry called surface enhanced laser desorption and ionization time of flight (SELDI-TOF) for the testing of ovarian cancer has led to an increased interest in proteomics as a diagnostic tool (Petricoin E F, et al). Furthermore, proteomics has been applied to the study of breast cancer through use of 2D gel electrophoresis and image analysis to study the development and progression of breast carcinoma in patients (Kuerer, H M, et al.).

Detection of biomarker molecules is an active field of research. For example, U.S. Pat. No. 5,958,785 discloses a biomarker for detecting long-term or chronic alcohol consumption. The biomarker disclosed is a single biomarker and is identified as an alcohol-specific ethanol glycoconjugate. U.S. Pat. No. 6,124,108 discloses a biomarker for mustard chemical injury. The biomarker is a specific protein band detected through gel electrophoresis and the patent describes use of the biomarker to produce protective antibodies in a kit to identify the presence or absence of the biomarker in individuals who may have been exposed to mustard poisoning. U.S. Pat. No. 6,326,209 discloses measurement of total urinary 17 ketosteroid-sulfates as biomarkers of biological age. U.S. Pat. No. 6,693,177 discloses a process for preparation of a single biomarker specific for 0-2 acetylated sialic acid and useful for diagnosis and outcome monitoring in patients with lymphoblastic leukemia.

Neurodegenerative diseases such as Alzheimer's disease (AD) are difficult to diagnose, particularly in their earlier stages. Currently there are no biomarkers in blood available for early diagnosis, differential diagnosis, determination and monitoring of disease severity and mechanisms, or for use as drug targets for treatment of neurodegenerative diseases such as Alzheimer's disease.

Therefore, there remains a need for better ways to objectively and accurately detect, diagnose, and distinguish AD from other neurodegenerative diseases, to accurately and specifically diagnose patients, to predict therapeutic response, to stratify patients for clinical trials, to measure disease severity, to monitor patient's response to treatment, and to find new drug targets to design new drugs.

In Alzheimer's disease, one genetic abnormality, the dementia risk Apo E ε4 gene allele, is inherited as one of three Apo E alleles, termed ε2, ε3, and ε4, with mean frequencies in the general population of about 8%, 78%, and 14%, respectively (Utermann G, et al.). The degree of risk of dementia conferred by the Apo E ε4 allele rises in a “gene dose” dependent manner (Corder, E. H. et al.), increasing with the number of Apo E ε4 alleles inherited, from: zero, i.e. Apo E ε4 non-carriers; to carriers of one Apo E ε4 allele, i.e. ε4/ε3; ε4/ε2 hetero-zygotes; to two Apo E ε4 alleles, i.e. zygotes (Greenwood P M, et al.), all of whom are capable of developing Alzheimer's disease, although those lacking the Apo E ε4 allele may tend to get the disease at a later age of onset (Poirier J, J.).

### SUMMARY OF THE INVENTION

The present invention relates to blood serum protein biomarkers for Alzheimer's disease, whereby the detection and/or concentration, or the lack of detection of one or more proteins correlates with significant increases or decreases in one or more other proteins in a disease specific manner. More specifically, the present invention relates to blood serum protein biomarkers for Alzheimer's disease, whereby the detection, and/or concentration, or the lack of detection, of a first biomarker such as an Apolipoprotein E4 protein in the blood serum of Alzheimer's disease patients correlates with significant differences in the blood serum concentrations of additional protein biomarkers of Alzheimer's disease, such as an Apolipoprotein E3 protein, an Apolipoprotein A-IV Protein, a Transthyretin protein, a Complement C3c1 protein, a Complement C3c2a protein, a Complement C3dg protein, a Complement Factor Bb protein, a Complement Factor H/Hs protein, a Complement Factor I protein, an Immunoglobulin protein, a Haptoglobin protein, and/or an Inter-alpha Trypsin Inhibitor Heavy Chain (H4) related 35 KD protein. Also in the present invention, Alzheimer's disease patients, and age-matched normal control subjects, are each placed into separate categories based on whether they do or do not have detectable blood serum levels of a first biomarker such as an Apolipoprotein E4 protein, and the differences in these and other Alzheimer's disease blood serum biomarker protein profiles indicate differences in Alzheimer's disease mechanisms, providing opportunities for improvements in differential diagnosis, disease severity and drug response monitoring, drug clinical trial stratification of patients, and for discovery of new targeted therapies.

One aspect of the present invention is the use of blood serum protein biomarkers for screening, diagnosis, differential diagnosis, and determining and monitoring of disease severity and mechanisms of Alzheimer's disease, comprising obtaining a blood serum sample from a test subject; determining whether a quantity of an Apolipoprotein E4 protein can be detected in the blood serum sample, wherein detection of a quantity of a first protein biomarker such as an Apolipoprotein E4 protein in the test subject blood serum sample is indicative of one form of Alzheimer's disease or a normal condition with a potential to develop that form of Alzheimer's disease, and the lack of detection of a quantity of a first protein biomarker such as an Apolipoprotein E4 protein in the test subject blood serum sample is indicative of another form of Alzheimer's disease or a normal condition with a potential to develop the other form of Alzheimer's disease.

Yet another aspect of the present invention is the use of the blood serum protein biomarkers for screening, diagnosis, or differential diagnosis of Alzheimer's disease comprising obtaining a blood serum sample from a test subject; determining whether or not a quantity of a first protein biomarker such as an Apolipoprotein E4 protein can be detected in the blood serum sample; and determining the quantity of the first protein biomarker such as an Apolipoprotein E4 protein, and of one or more additional protein biomarkers such as an Apolipoprotein E3 protein, an Apolipoprotein A-IV Protein, a Transthyretin protein, a Complement C3c1 protein, a Complement C3c2a protein, a complement C3dg protein, a Complement Factor Bb protein, a Complement Factor H/Hs protein, a Complement Factor I protein, an Immunoglobulin protein, a Haptoglobin protein, and/or an Inter-alpha Trypsin Inhibitor Heavy Chain (H4) related 35 KD protein in the blood serum sample, and determining whether the first protein biomarker such as an Apolipoprotein E4 protein can be detected and determining the quantities of a first protein biomarker such as an Apolipoprotein E4 protein, and of one or more additional protein biomarkers such as an Apolipoprotein E3 protein, an Apolipoprotein A-IV Protein, a Transthyretin protein, a Complement C3c1 protein, a Complement C3c2a protein, a complement C3dg protein, a Complement Factor Bb protein, a Complement Factor H/Hs protein, a Complement Factor I protein, an Immunoglobulin protein, a Haptoglobin protein, and/or an Inter-alpha Trypsin Inhibitor Heavy Chain (H4) related 35 KD protein in blood serum samples from normal control individuals, from patients with Alzheimer's disease, with Parkinson's disease, and with AD-Like and Mixed dementias, wherein the detection of the first protein biomarker such as an Apolipoprotein E4 protein in the test subject blood serum sample is indicative of one form of Alzheimer's disease or a normal condition with a potential to develop that form of Alzheimer's disease, and the quantity of the first protein biomarker such as an Apolipoprotein E4 protein, and of one or more additional protein biomarkers such as an Apolipoprotein E3 protein, an Apolipoprotein A-IV Protein, a Transthyretin protein, a Complement C3c1 protein, a Complement C3c2a protein, a complement C3dg protein, a Complement Factor Bb protein, a Complement Factor H/Hs protein, a Complement Factor I protein, an Immunoglobulin protein, a Haptoglobin protein, and/or an Inter-alpha Trypsin Inhibitor Heavy Chain (H4) related 35 KD protein in the blood serum sample of the test subject within the ranges of that form of Alzheimer's disease values is indicative of the presence of that form of Alzheimer's disease, and the quantity of the first protein biomarker such as an Apolipoprotein E4 protein, and of one or more additional protein biomarkers such as an Apolipoprotein E3 protein, an Apolipoprotein A-IV Protein, a Transthyretin protein, a Complement C3c1 protein, a Complement C3c2a protein, a complement C3dg protein, a Complement Factor Bb protein, a Complement Factor H/Hs protein, a Complement Factor I protein, an Immunoglobulin protein, a Haptoglobin protein, and/or an Inter-alpha Trypsin Inhibitor Heavy Chain (H4) related 35 KD protein in the blood serum sample of the test subject outside the range of that form of Alzheimer's disease values are indicative of the absence of that form of Alzheimer's disease and the presence of a normal condition, or another neurological disorder, such as Parkinson's disease, or an AD-Like or Mixed dementia, such as: Frontotemporal dementia (FTD); Lewy body dementia (LBD); Alcohol related dementia; Semantic dementia; Vascular (Multi-infarct) dementia; Stroke (CVA); Post-irradiation Encephalopathy and Seizures; Alzheimer's disease combined with Vascular (Multi-Infarct) dementia; Alzheimer's disease combined with Lewy body dementia; Parkinson's disease combined with Lewy body dementia; Alzheimer's and Parkinson's disease combined with Lewy body dementia; Frontotemporal dementia combined with Chronic Inflammatory Demyelinating Polyneuropathy; and Thalamic CVA combined with HX of Lung CA, and wherein the lack of detection of a quantity of the first protein biomarker such as an Apolipoprotein E4 protein in the test subject blood serum sample is indicative of another form of Alzheimer's disease or a normal condition with a potential to develop that other form of Alzheimer's disease, and the quantity of the first protein biomarker such as an Apolipoprotein E4 protein, and of one or more additional protein biomarkers such as an Apolipoprotein E3 protein, an Apolipoprotein A-IV Protein, a Transthyretin protein, a Complement C3c1 protein, a Complement C3c2a protein, a complement C3dg protein, a Complement Factor Bb protein, a Complement Factor H/Hs protein, a Complement Factor I protein, an Immunoglobulin protein, a Haptoglobin protein, and/or an Inter-alpha Trypsin Inhibitor Heavy Chain (H4) related 35 KD protein in the blood serum sample of the test subject within the ranges of that other form of Alzheimer's disease values is indicative of the presence of that other form of Alzheimer's disease, and the quantity of the first protein biomarker such as an Apolipoprotein E4 protein, and of one or more additional protein biomarkers such as an Apolipoprotein E3 protein, an Apolipoprotein A-IV Protein, a Transthyretin protein, a Complement C3c1 protein, a Complement C3c2a protein, a complement C3dg protein, a Complement Factor Bb protein, a Complement Factor H/Hs protein, a Complement Factor I protein, an Immunoglobulin protein, a Haptoglobin protein, and/or an Inter-alpha Trypsin Inhibitor Heavy Chain (H4) related 35 KD protein in the blood serum sample of the test subject outside the range of that other form of Alzheimer's disease values are indicative of the absence of that form of Alzheimer's disease and the presence of a normal condition, or another neurological disorder, such as Parkinson's disease, or an AD-Like or Mixed dementia, such as: Frontotemporal dementia (FTD); Lewy body dementia (LBD); Alcohol related dementia; Semantic dementia; Vascular (Multi-infarct) dementia; Stroke (CVA); Post-irradiation Encephalopathy and Seizures; Alzheimer's disease combined with Vascular (Multi-Infarct) dementia; Alzheimer's disease combined with Lewy body dementia; Parkinson's disease combined with Lewy body dementia; Alzheimer's and Parkinson's disease combined with Lewy body dementia; Frontotemporal dementia combined with Chronic Inflammatory Demyelinating Polyneuropathy; and Thalamic CVA combined with HX of Lung CA.

Yet another aspect of the present invention is the use of the blood serum protein biomarkers for screening, diagnosis, or differential diagnosis of Alzheimer's disease comprising obtaining a blood serum sample from a test subject; determining whether or not an Apolipoprotein E4 protein can be detected in the blood serum sample; and determining the quantity of a first protein biomarker such as Apolipoprotein E4 protein, and of one or more additional protein biomarkers such as an Apolipoprotein E3 protein, an Apolipoprotein A-IV Protein, a Transthyretin protein, a Complement C3c1 protein, a Complement C3c2a protein, a complement C3dg protein, a Complement Factor Bb protein, a Complement Factor H/Hs protein, a Complement Factor I protein, an Immunoglobulin protein, a Haptoglobin protein, and/or an Inter-alpha Trypsin Inhibitor Heavy Chain (H4) related 35 KD protein in the blood serum sample, by quantitative two-dimensional gel electrophoresis; and determining whether a quantity of the first protein biomarker such as an Apolipoprotein E4 protein can be detected, and quantitating the first protein biomarker such as an Apolipoprotein E4 protein, and of one or more additional protein biomarkers such as an Apolipoprotein E3 protein, an Apolipoprotein A-IV Protein, a Transthyretin protein, a Complement C3c1 protein, a Complement C3c2a protein, a complement C3dg protein, a Complement Factor Bb protein, a Complement Factor H/Hs protein, a Complement Factor I protein, an Albumin protein, a Haptoglobin protein, and/or an Inter-alpha Trypsin Inhibitor Heavy Chain (H4) related 35 KD protein in the protein expression patterns of the 2D gels of the serum samples; wherein the detection of the first protein biomarker such as an Apolipoprotein E4 protein in the test subject blood serum sample is indicative of one form of Alzheimer's disease or a normal condition with a potential to develop that form of Alzheimer's disease, and the quantity of the first protein biomarker such as an Apolipoprotein E4 protein, and of one or more additional protein biomarkers such as an Apolipoprotein E3 protein, an Apolipoprotein A-IV Protein, a Transthyretin protein, a Complement C3c1 protein, a Complement C3c2a protein, a complement C3dg protein, a Complement Factor Bb protein, a Complement Factor H/Hs protein, a Complement Factor I protein, an Immunoglobulin protein, a Haptoglobin protein, and/or an Inter-alpha Trypsin Inhibitor Heavy Chain (H4) related 35 KD protein in the blood serum sample of the test subject within the ranges of that form of Alzheimer's disease values is indicative of the presence of that form of Alzheimer's disease, and the quantity of the first protein biomarker such as an Apolipoprotein E4 protein, and of one or more additional protein biomarkers such as an Apolipoprotein E3 protein, an Apolipoprotein A-IV Protein, a Transthyretin protein, a Complement C3c1 protein, a Complement C3c2a protein, a complement C3dg protein, a Complement Factor Bb protein, a Complement Factor H/Hs protein, a Complement Factor I protein, an Immunoglobulin protein, a Haptoglobin protein, and/or an Inter-alpha Trypsin Inhibitor Heavy Chain (H4) related 35 KD protein in the blood serum sample of the test subject outside the range of that form of Alzheimer's disease values are indicative of the absence of that form of Alzheimer's disease and the presence of a normal condition, or another neurological disorder, such as Parkinson's disease, or an AD-Like or Mixed dementia, and wherein the lack of detection of a quantity of the first protein biomarker such as an Apolipoprotein E4 protein in the test subject blood serum sample is indicative of another form of Alzheimer's disease or a normal condition with a potential to develop that other form of Alzheimer's disease, and the quantity of the first protein biomarker such as an Apolipoprotein E4 protein, and of one or more additional protein biomarkers such as an Apolipoprotein E3 protein, an Apolipoprotein A-IV Protein, a Transthyretin protein, a Complement C3c1 protein, a Complement C3c2a protein, a complement C3dg protein, a Complement Factor Bb protein, a Complement Factor H/Hs protein, a Complement Factor I protein, an immunoglobulin protein, a Haptoglobin protein, and/or an Inter-alpha Trypsin Inhibitor Heavy Chain (H4) related 35 KD protein in the blood serum sample of the test subject within the ranges of that other form of Alzheimer's disease values is indicative of the presence of that other form of Alzheimer's disease, and the quantity of the first protein biomarker such as an Apolipoprotein E4 protein, and of one or more additional protein biomarkers such as an Apolipoprotein E3 protein, an Apolipoprotein A-IV Protein, a Transthyretin protein, a Complement C3c1 protein, a Complement C3c2a protein, a complement C3dg protein, a Complement Factor Bb protein, a Complement Factor H/Hs protein, a Complement Factor I protein, an Immunoglobulin protein, a Haptoglobin protein, and/or an Inter-alpha Trypsin Inhibitor Heavy Chain (H4) related 35 KD protein in the blood serum sample of the test subject outside the range of that other form of Alzheimer's disease values are indicative of the absence of that form of Alzheimer's disease and the presence of a normal condition, or another neurological disorder, such as Parkinson's disease, or an AD-Like or Mixed dementia, such as: Frontotemporal dementia (FTD); Lewy body dementia (LBD); Alcohol related dementia; Semantic dementia; Vascular (Multi-infarct) dementia; Stroke (CVA); Post-irradiation Encephalopathy and Seizures; Alzheimer's disease combined with Vascular (Multi-Infarct) dementia; Alzheimer's disease combined with Lewy body dementia; Parkinson's disease combined with Lewy body dementia; Alzheimer's and Parkinson's disease combined with Lewy body dementia; Frontotemporal dementia combined with Chronic Inflammatory Demyelinating Polyneuropathy; and Thalamic CVA combined with HX of Lung CA.

Yet another aspect of the present invention is the use of the blood serum protein biomarkers for screening, diagnosis, or differential diagnosis of Alzheimer's disease comprising obtaining a blood serum sample from a test subject; determining whether or not a first protein biomarker such as an Apolipoprotein E4 protein can be detected in the blood serum sample; and determining the quantity of the first protein biomarker such as an Apolipoprotein E4 protein, and of one or more additional protein biomarkers such as an Apolipoprotein E3 protein, an Apolipoprotein A-IV Protein, a Transthyretin protein, a Complement C3c1 protein, a Complement C3c2a protein, a complement C3dg protein, a Complement Factor Bb protein, a Complement Factor H/Hs protein, a Complement Factor I protein, an immunoglobulin protein, a Haptoglobin protein, and/or an Inter-alpha Trypsin Inhibitor Heavy Chain (H4) related 35 KD protein in the blood serum sample, by an immunoassay using an antibody that recognizes the first protein biomarker such as an Apolipoprotein E4 protein and one or more other antibodies that recognize one or more additional protein biomarkers such as an Apolipoprotein E4 protein, an Apolipoprotein E3 protein, an Apolipoprotein A-IV Protein, a Transthyretin protein, a Complement C3c1 protein, a Complement C3c2a protein, a complement C3dg protein, a Complement Factor Bb protein, a Complement Factor H/Hs protein, a Complement Factor I protein, an immunoglobulin protein, a Haptoglobin protein, and/or an Inter-alpha Trypsin Inhibitor Heavy Chain (H4) related 35 KD protein, in the blood serum sample of the test subject, wherein the detection of the first protein biomarker such as an Apolipoprotein E4 protein in the test subject blood serum sample is indicative of one form of Alzheimer's disease or a normal condition with a potential to develop that form of Alzheimer's disease, and the quantity of the first protein biomarker such as an Apolipoprotein E4 protein, and of additional protein biomarkers such as an Apolipoprotein E3 protein, an Apolipoprotein A-IV Protein, a Transthyretin protein, a Complement C3c1 protein, a

Complement C3c2a protein, a complement C3dg protein, a Complement Factor Bb protein, a Complement Factor H/Hs protein, a Complement Factor I protein, an Immunoglobulin protein, a Haptoglobin protein, and/or an Inter-alpha Trypsin Inhibitor Heavy Chain (H4) related 35 KD protein in the blood serum sample of the test subject within the ranges of that form of Alzheimer's disease values is indicative of the presence of that form of Alzheimer's disease, and the quantity of the first protein biomarker such as an Apolipoprotein E4 protein, and of additional protein biomarkers such as an Apolipoprotein E3 protein, an Apolipoprotein A-IV Protein, a Transthyretin protein, a Complement C3c1 protein, a Complement C3c2a protein, a complement C3dg protein, a Complement Factor Bb protein, a Complement Factor H/Hs protein, a Complement Factor I protein, an Immunoglobulin protein, a Haptoglobin protein, and/or an Inter-alpha Trypsin Inhibitor Heavy Chain (H4) related 35 KD protein in the blood serum sample of the test subject outside the range of that form of Alzheimer's disease values are indicative of the absence of that form of Alzheimer's disease and the presence of a normal condition, or another neurological disorder, such as Parkinson's disease, or an AD-Like or Mixed dementia, such as: Frontotemporal dementia (FTD); Lewy body dementia (LBD); Alcohol related dementia; Semantic dementia; Vascular (Multi-infarct) dementia; Stroke (CVA); Post-irradiation Encephalopathy and Seizures; Alzheimer's disease combined with Vascular (Multi-Infarct) dementia; Alzheimer's disease combined with Lewy body dementia; Parkinson's disease combined with Lewy body dementia; Alzheimer's and Parkinson's disease combined with Lewy body dementia; Frontotemporal dementia combined with Chronic Inflammatory Demyelinating Polyneuropathy; and Thalamic CVA combined with HX of Lung CA, and wherein the lack of detection of a quantity of the first protein biomarker such as an Apolipoprotein E4 protein in the test subject blood serum sample is indicative of another form of Alzheimer's disease or a normal condition with a potential to develop that other form of Alzheimer's disease, and the quantity of the first protein biomarker such as an Apolipoprotein E4 protein, and of additional protein biomarkers such as an Apolipoprotein E3 protein, an Apolipoprotein A-IV Protein, a Transthyretin protein, a Complement C3c1 protein, a Complement C3c2a protein, a complement C3dg protein, a Complement Factor Bb protein, a Complement Factor H/Hs protein, a Complement Factor I protein, an Immunoglobulin protein, a Haptoglobin protein, and/or an Inter-alpha Trypsin Inhibitor Heavy Chain (H4) related 35 KD protein in the blood serum sample of the test subject within the ranges of that other form of Alzheimer's disease values is indicative of the presence of that other form of Alzheimer's disease, and the quantity of the first protein biomarker such as an Apolipoprotein E4 protein, and of additional protein biomarkers such as an Apolipoprotein E3 protein, an Apolipoprotein A-IV Protein, a Transthyretin protein, a Complement C3c1 protein, a Complement C3c2a protein, a complement C3dg protein, a Complement Factor Bb protein, a Complement Factor H/Hs protein, a Complement Factor I protein, an Immunoglobulin protein, a Haptoglobin protein, and/or an Inter-alpha Trypsin Inhibitor Heavy Chain (H4) related 35 KD protein in the blood serum sample of the test subject outside the range of that other form of Alzheimer's disease values are indicative of the absence of that form of Alzheimer's disease and the presence of a normal condition, or another neurological disorder, such as Parkinson's disease, or an AD-Like or Mixed dementia, such as: Frontotemporal dementia (FTD); Lewy body dementia (LBD); Alcohol related dementia; Semantic dementia; Vascular (Multi-infarct) dementia; Stroke (CVA); Post-irradiation Encephalopathy and Seizures; Alzheimer's disease combined with Vascular (Multi-Infarct) dementia; Alzheimer's disease combined with Lewy body dementia; Parkinson's disease combined with Lewy body dementia; Alzheimer's and Parkinson's disease combined with Lewy body dementia; Frontotemporal dementia combined with Chronic Inflammatory Demyelinating Polyneuropathy; and Thalamic CVA combined with HX of Lung CA.

Yet another aspect of the present invention is the use of blood serum protein biomarkers, for early detection and for monitoring the disease severity and response to therapy of patients with Alzheimer's disease, comprising obtaining a blood serum sample from a test subject; determining whether a first protein biomarker such as an Apolipoprotein E4 protein can be detected and determining the quantity of the first protein biomarker such as an Apolipoprotein E4 protein, and of additional protein biomarkers such as an Apolipoprotein E3 protein, an Apolipoprotein A-IV Protein, a Transthyretin protein, a Complement C3c1 protein, a Complement C3c2a protein, a complement C3dg protein, a Complement Factor Bb protein, a Complement Factor H/Hs protein, a Complement Factor I protein, an Immunoglobulin protein, a Haptoglobin protein, and/or an Inter-alpha Trypsin Inhibitor Heavy Chain (H4) related 35 KD protein in the blood serum sample from the test subject, and in blood serum samples from normal control individuals, and from patients with mild (MMSE score=25-20), moderate (MMSE score=19-11) and severe (MMSE≦10) Alzheimer's disease, wherein, whether an Apolipoprotein E4 protein can be detected and the quantity of an Apolipoprotein E4 protein, and of additional protein biomarkers such as an the first protein biomarker such as Apolipoprotein E3 protein, an Apolipoprotein A-IV Protein, a Transthyretin protein, a Complement C3c1 protein, a Complement C3c2a protein, a complement C3dg protein, a Complement Factor Bb protein, a Complement Factor H/Hs protein, a Complement Factor I protein, an Immunoglobulin protein, a Haptoglobin protein, and/or an Inter-alpha Trypsin Inhibitor Heavy Chain (H4) related 35 KD protein in the blood serum sample from the test subject, indicates the degree of severity of Alzheimer's disease in the test subject.

The foregoing has outlined rather broadly several aspects of the present invention in order that the detailed description of the invention that follows may be better understood. Additional features and advantages of the invention will be described hereinafter which form the subject of the claims of the invention. It should be appreciated by those skilled in the art that the conception and the specific embodiment disclosed might be readily utilized as a basis for modifying or redesigning the structures for carrying out the same purposes as the invention. It should be realized by those skilled in the art that such equivalent constructions do not depart from the spirit and scope of the invention as set forth in the appended claims.

### BRIEF DESCRIPTION OF THE DRAWINGS

For a more complete understanding of the present invention, and the advantages thereof, reference is now made to the following descriptions taken in conjunction with the accompanying drawings, in which:

FIGS. 1A-1B illustrate the dynamic range of the assay and the reproducibility within triplicate assays of quantitative 2D gel electrophoresis of human blood serum. Shown in FIG. 1A are several protein spots (circles) within the 2D gel pattern of human blood serum, with spot concentrations ranging from 55 ppm to 15,789 ppm (white arrows). FIG. 1B shows triplicate analysis (details of three 2D gels run with the same blood serum sample) with a coefficient of variation=of 13.8% for the triplicate analysis of the individual spot concentrations. See also Table 2 for reproducibility over the dynamic range.

FIG. 2 illustrates the location (circles and numbers) of biomarker protein spots within a 2D Gel electrophoresis protein expression profile of human blood serum, namely Apolipoprotein E4 protein spot N5302; Apolipoprotein E3 protein spot N3314; Transthyretin “Dimer” protein spot N3307; Complement C3c1 protein spot N7310; Complement C3c2a protein spot N9311; Complement C3dg protein spot N1511; Complement Factor Bb protein spot N7616; Complement Factor H/Hs protein spot N4411; Inter alpha Trypsin Inhibitor Heavy Chain H4 related 35 KD protein spot N2307; Immunoglobulin Light Chain protein spot N6224; Apolipoprotein A-IV protein spot N2502; Complement Factor I protein spot N1416: and Haptoglobin protein spots N1514, N2401, N2407, and N3409. These spots are among the differentially expressed proteins detected in 2D gels of blood serum collected from normal subjects, patients with neurodegenerative diseases and patients with like-disease disorders, where the indicated protein spots were identified by LC-MS/MS analysis of in-gel trypsin digests of the spots.

FIG. 3A is a comparative statistical Dot, Box and Whiskers graph (constructed using Analyze-it software for Microsoft Excel, illustrating the differential expression level (PPM) of an Apolipoprotein E4 protein spot N5302 in blood serum, based on the quantitative 2D gel triplicate analysis data obtained with blood serum samples from 75 normal control individuals (Controls), 115 Alzheimer's disease patients (AD), 12 Parkinson's disease patients (PD), and 12 patients with AD-Like and Mixed disorders, including Frontotemporal dementia (FTD), Frontotemporal dementia combined with Chronic Inflammatory Demyelinating polyneuropathy, Lewy body dementia (LBD), Vascular (Multi-infarct) dementia, Thalamic CVA combined with HX or Lung CA, Post-irradiation Encephalopathy, Seizures, Alcohol related dementia, Semantic dementia, Memory Dysfunction, Neuro Exam “Normal,” and Corticalbasal Ganglionic Degeneration (CBGD), with examples of the differences between the ranges of the patient and control groups and their statistical significance by analysis of variance (ANOVA-P). FIG. 3B shows receiver Operating Characteristic (ROC) curve of Apolipoprotein E4 spot N5302 when used as a single biomarker to differentiate between Alzheimer's disease patients and age-matched control (AMC) subjects with an area under the curve (AUC) of 0.66±0.02, sensitivity, specificity, and ROC probability (ROC-P), calculated by Analyze-it for Microsoft Excel with these data.

FIG. 4A shows statistical Dot, Box and Whiskers graph constructed using Analyze-it software for Microsoft Excel, illustrating the differential expression level (PPM) of a Apolipoprotein E4 protein spot N5302 in blood serum, based on the quantitative 2D gel triplicate analysis data (dots) obtained with blood serum samples from age matched normal controls, and patients with Alzheimer's disease, (Total N5302 and N5302>0) constructed using Analyze-it software for Microsoft Excel. Blood serum samples were from: 75 Age matched normal control individuals (Controls); of which 23 Age matched normal control individuals (31%) had detectable quantities of Apolipoprotein E4 protein spot N5302 (N5302>0) in their blood serum; and 115 Alzheimer's disease patients (AD); of which 67 Alzheimer's disease patients (58%), had detectable quantities of Apolipoprotein E4 protein spot N5302 (N5302>0) in their blood serum. FIG. 4B is Receiver Operating Characteristic (ROC) curve of Apolipoprotein E4 spot N5302 from populations where the biomarker level (ppm) was greater than zero (N5302>0) was used as a single biomarker to differentiate between 67 Alzheimer's disease (AD) patients and 23 age-matched control (AMC) subjects with an area under the curve (AUC) of 0.61±0.04.

FIG. 5A is a comparative statistical Box and Whiskers graph constructed using Analyze-it software for Microsoft Excel, illustrating the differential expression level (PPM) of an Apolipoprotein E3 protein spot N3314 in blood serum, based on the quantitative 2D gel triplicate analysis data obtained with blood serum samples from: 75 normal control individuals (Controls), 115 Alzheimer's disease patients (AD), 12 Parkinson's disease patients (PD), and 12 patients with AD-Like and Mixed disorders, including: Frontotemporal dementia (FTD), Frontotemporal dementia combined with Chronic Inflammatory Demyelinating polyneuropathy, Lewy body dementia (LBD), Vascular (Multi-infarct) dementia, Thalamic CVA combined with HX or Lung CA, Post-irradiation Encephalopathy, Seizures, Alcohol related dementia, Semantic dementia, Memory Dysfunction, neuro exam “Normal,” and Corticalbasal Ganglionic Degeneration (CBGD), with examples of the differences between the ranges of the patient and control groups and their statistical significance by analysis of variance (ANOVA-P). FIG. 5B is a Receiver Operating Characteristic (ROC) curve of Apolipoprotein E3 spot N3314 when used as a single biomarker to differentiate between Alzheimer's disease (AD) patients and age-matched control (AMC) subjects with an area under the curve (AUC) of 0.71±0.022, sensitivity, specificity, and ROC probability (ROC-P), calculated by Analyze-it for Microsoft Excel with these data.

FIG. 6 shows a comparative statistical Box and Whiskers graph (constructed using Analyze-it software for Microsoft Excel), illustrating the differential expression level (PPM) of an Apolipoprotein E3 protein spot N3314 in blood serum, based on the quantitative 2D gel triplicate analysis data obtained with blood serum samples from patients with Alzheimer's disease (AD) and age matched normal control (AMC) subjects, when Apolipoprotein E4 (spot N5302) was not detected (N5302=0, left panel) and when it was detected (N5302>0, right panel) in the 2D gels of their blood serum.

FIG. 7A shows a plot of the Receiver Operator Characteristics (ROC) curve (calculated by using Analyse-it software for Microsoft Excel) of blood serum concentrations of Apolipoprotein E3 protein spot N3314 when used to distinguish between patients with Alzheimer's disease (AD) and age matched normal controls (AMC) as a function of whether Apolipoprotein E4 protein (spot N5302) is detected (N5302>0) or not detected (N5302=0) in blood serum. FIG. 7B is a Receiver Operator Characteristics (ROC) curve of blood serum concentrations of Apolipoprotein E3 protein (spot N3314) when used to distinguish between two Alzheimer's disease (AD) groups as a function of whether Apolipoprotein E4 protein (spot N5302) is detected (N5302>0) or not detected (N5302=0) in blood serum.

FIG. 8A is Dot, Box and Whiskers graph constructed using Analyze-it software for Microsoft Excel, illustrating the differential expression level (PPM) of Transthyretin “Dimer” protein spot N3307 in blood serum, based on the quantitative 2D gel triplicate analysis data obtained with blood serum samples from: 75 normal control individuals (Controls), 115 Alzheimer's disease patients (AD), 12 Parkinson's disease patients (PD), and 12 patients with AD-Like and Mixed disorders, including: Frontotemporal dementia (FTD), Frontotemporal dementia combined with Chronic Inflammatory Demyelinating polyneuropathy, Lewy body dementia (LBD), Vascular (Multi-infarct) dementia, Thalamic CVA combined with HX or Lung CA, Post-irradiation Encephalopathy, Seizures, Alcohol related dementia, Semantic dementia, Memory Dysfunction, neuro exam “Normal,” and Corticalbasal Ganglionic Degeneration (CBGD), with examples of the differences between the ranges of the patient and control groups and their statistical significance by analysis of variance (ANOVA-P). FIG. 8B is Receiver Operating Characteristic (ROC) curve of Transthyretin “Dimer” spot N3307 when used as a single biomarker to differentiate between Alzheimer's disease (AD) patients and age-matched control (AMC) subjects with an area under the curve (AUC) of 0.66±0.023, sensitivity, specificity, and ROC probability (ROC-P), calculated by Analyze-it for Microsoft Excel with these data.

FIG. 9A is a statistical Box and Whiskers graph (constructed using Analyze-it software for Microsoft Excel), illustrating the differential expression level (PPM) of Transthyretin “Dimer” protein spot N3307 in blood serum, based on the quantitative 2D gel triplicate analysis data obtained with blood serum samples from age matched normal controls (AMC), and patients with Alzheimer's disease. FIG. 9B is a Receiver Operating Characteristic (ROC) curve of Transthyretin (spot N3307) when used to distinguish between patients with Alzheimer's disease (AD) and age-matched control (AMC) subjects as a function of Apolipoprotein E4 spot N5302 when not detected (N5302=0) and when detected (N5302>0) in the 2D gels of their blood serum.

FIG. 10A is a statistical Dot, Box and Whiskers graph (constructed using Analyze-it software for Microsoft Excel), illustrating the differential expression level (PPM) of Complement Factor H/Hs protein spot N4411 in blood serum, based on the quantitative 2D gel triplicate analysis data obtained with blood serum samples from: 75 normal control individuals (Controls), 115 Alzheimer's disease patients (AD), 12 Parkinson's disease patients (PD), and 12 patients with AD-Like and Mixed disorders, including: Frontotemporal dementia (FTD), Frontotemporal dementia combined with Chronic Inflammatory Demyelinating polyneuropathy, Lewy body dementia (LBD), Vascular (Multi-infarct) dementia, Thalamic CVA combined with HX or Lung CA, Post-irradiation Encephalopathy, Seizures, Alcohol related dementia, Semantic dementia, Memory Dysfunction, neuro exam “Normal,” and Corticalbasal Ganglionic Degeneration (CBGD), with examples of the differences between the ranges of the patient and control groups and their statistical significance by analysis of variance (ANOVA-P). FIG. 10B is a Receiver Operating Characteristic (ROC) curve of Complement factor H/Hs protein spot N4411 when used as a single biomarker to differentiate between Alzheimer's disease (AD) patients and age-matched control (AMC) subjects with an area under the curve (AUC) of 0.59±0.024 sensitivity, specificity, and ROC probability (ROC-P), calculated by Analyze-it for Microsoft Excel with these data.

FIG. 11A is a statistical Dot, Box and Whiskers graph (constructed using Analyze-it software for Microsoft Excel), illustrating the differential expression level (PPM) of Complement Factor H/Hs protein (spot N4411) in blood serum, based on the quantitative 2D gel triplicate analysis data obtained with blood serum samples from age matched normal controls (AMC), and patients with Alzheimer's disease. FIG. 11B is a Receiver Operating Characteristic (ROC) curve of Complement Factor H/Hs protein (spot N4411) when used to distinguish between Patients with Alzheimer's disease (AD) and age-matched control (AMC) subjects as a function of Apolipoprotein E4 (spot N5302) when not detected (N5302=0) and when detected (N5302>0) in the 2D gels of their blood serum.

FIG. 12A is a statistical Dot, Box and Whiskers graph (constructed using Analyze-it software for Microsoft Excel), illustrating the differential expression level (PPM) of Complement Factor Bb protein (spot N7616) in blood serum, based on the quantitative 2D gel triplicate analysis data obtained with blood serum samples from: 75 normal control individuals (Controls), 115 Alzheimer's disease patients (AD), 12 Parkinson's disease patients (PD), and 12 patients with AD-Like and Mixed disorders, including: Frontotemporal dementia (FTD), Frontotemporal dementia combined with Chronic Inflammatory Demyelinating polyneuropathy, Lewy body dementia (LBD), Vascular (Multi-infarct) dementia, Thalamic CVA combined with HX or Lung CA, Post-irradiation Encephalopathy, Seizures, Alcohol related dementia, Semantic dementia, Memory Dysfunction, neuro exam “Normal,” and Corticalbasal Ganglionic Degeneration (CBGD), with examples of the differences between the ranges of the patient and control groups and their statistical significance by analysis of variance (ANOVA-P). FIG. 12B is a Receiver Operating Characteristic (ROC) curve of Factor Bb protein (spot N7616) when used as a single biomarker to differentiate between Alzheimer's disease (AD) patients and age-matched control (AMC) subjects with an area under the curve (AUC) of 0.53±0.024 sensitivity, specificity, and ROC probability (ROC-P), calculated by Analyze-it for Microsoft Excel with these data.

FIG. 13A is a statistical Dot, Box and Whiskers graph (constructed using Analyze-it software for Microsoft Excel), illustrating the differential expression level (PPM) of Complement Factor Bb protein (spot N7616) in blood serum, based on the quantitative 2D gel triplicate analysis data obtained with blood serum samples from age matched normal controls (AMC), and patients with Alzheimer's disease. FIG. 13B is a Receiver Operating Characteristic (ROC) curve of Complement Factor Bb protein (spot N7616) when used to distinguish between patients with Alzheimer's disease (AD) and age-matched control (AMC) subjects as a function of Apolipoprotein E4 (spot N5302) when not detected (N5302=0) and when detected (N5302 >0) in the 2D gels of their blood serum.

FIGS. 14A-14D are statistical Box and Whiskers graphs (constructed using Analyze-it software for Microsoft Excel), illustrating the blood serum differential expression levels (PPM) of (FIG. 14A) Complement C3c1 phosphoprotein (spot N7310), (FIG. 14B) Complement C3dg protein spot N1511, derived from a different amino acid sequence of the C3 parent located just downstream of that shared by C3c1 protein spot N7310, and C3c2a protein spot N9311, (FIG. 14C) Complement C3c2a protein spot N9311, unphosphorylated form of Complement C3c1, and (FIG. 14D) the sum of the Complement C3c and C3dg proteins (N7310+N9311+N1511), based on the quantitative 2D gel triplicate analysis data obtained with blood serum samples from: 75 normal control individuals (Controls), 115 Alzheimer's disease patients (AD), 12 Parkinson's disease patients (PD), and 12 patients with AD-Like and Mixed disorders, including: Frontotemporal dementia (FTD), Frontotemporal dementia combined with Chronic Inflammatory Demyelinating polyneuropathy, Lewy body dementia (LBD), Vascular (Multi-infarct) dementia, Thalamic CVA combined with HX or Lung CA, Post-irradiation Encephalopathy, Seizures, Alcohol related dementia, Semantic dementia, Memory Dysfunction, neuro exam “Normal,” and Corticalbasal Ganglionic Degeneration (CBGD),with examples of the differences between the ranges of the patient and control groups and their statistical significance by analysis of variance (ANOVA-P) and by Sensitivity, Specificity, and ROC probability (ROC-P), calculated by Analyze-it for Microsoft Excel with these data.

FIGS. 15A-15D illustrate the Receiver Operator Characteristics (ROC) curves (constructed using Analyze-it software for Microsoft Excel), of: (FIG. 15A) Complement C3c1 phosphoprotein spot N7310, (FIG. 15B) Complement C3dg protein spot N1511, (derived from a different amino acid sequence of the C3 parent located just downstream of that shared by C3c1 protein spot N7310, and C3c2a protein spot N9311, (FIG. 15C) Complement C3c2a protein spot N9311, (unphosphorylated form of Complement C3c1), when each is used separately, and (FIG. 15D) the sum of the Complement C3c and C3dg proteins (N7310+N9311+N1511), to distinguish between patients with Alzheimer's disease (AD) and age-matched control (AMC) subjects.

FIGS. 16A-16D depicts Dot, Box and Whiskers graphs (constructed using Analyze-it software for Microsoft Excel), illustrating the blood serum differential expression levels (PPM) of: (FIG. 16A) Complement C3c1 phosphoprotein spot N7310, (FIG. 16B) Complement C3dg protein spot N1511, derived from a different amino acid sequence of the C3 parent located just downstream of that shared by C3c1 protein spot N7310 and C3c2a protein spot N9311, (FIG. 16C) Complement C3c2a protein spot N9311, unphosphorylated form of Complement C3c1, and (FIG. 16D) the sum of the Complement C3c and C3dg proteins (N7310+N9311+N1511), based on the quantitative 2D gel triplicate analysis data obtained with blood serum samples from age matched normal controls (AMC), and patients with Alzheimer's disease as a function of Apolipoprotein E4 protein spot N5302, when it is detected (N5302>0) or not detected (N5302=0) in the 2D gels of their blood serum.

FIGS. 17A-17D depicts the Receiver Operator Characteristics (ROCs) curves (constructed using Analyze-it software for Microsoft Excel) of (FIG. 17A) Complement C3dg protein spot N1511, derived from a different amino acid sequence of the C3 parent located just downstream of that shared by C3c1 phosphoprotein spot N7310, and C3c2a protein spot N9311, (FIG. 17B) Complement C3c1 phosphoprotein spot N7310 in blood serum, (FIG. 17C) Complement C3c2a protein spot N9311, unphosphorylated form of Complement C3c1, when each is used separately, and (FIG. 17D) the sum of the Complement C3c and C3dg proteins (N7310+N9311+N1511), to distinguish between patients with Alzheimer's disease (AD) and age matched normal control (AMC) subjects as a function of whether Apolipoprotein E4 protein spot N5302, when it is detected (N5302 >0) or not detected (N5302=0) in blood serum.

FIGS. 18A-18F depict the linear regression correlation of the blood serum expression level (PPM) of (FIG. 18A-18C) Complement C3c1 phosphoprotein spot N7310, and (FIG. 18D-18F) Complement C3c2a protein spot N9311, unphosphorylated form of Complement C3c1, with the severity of the Alzheimer's disease, measured clinically by Mini-Mental State Examination (MMSE) score, when the expression level of Apolipoprotein E4 (N5302) is detected (N5302>0; FIG. 18B, FIG. 18E) or not detected (N5302=0; FIG. 18A, FIG. 18D) in the blood serum. Severity of Alzheimer's disease increases with decreasing MMSE score (Mild: MMSE=25-20; Moderate: MMSE=19-11; Severe: MMSE≦10). A Box and Whisker graph (FIG. 18C, FIG. 18F) illustrate the comparative blood serum expression level (PPM) of Complement C3c1 N7310 in age-matched control (AMC) subjects. Linear regression and Box and Whisker graphs were constructed using Analyze-it software for Microsoft Excel.

FIGS. 19A-19C are statistical linear regression correlation of the blood serum expression level (PPM) of Complement C3dg protein spot N1511 with the severity of the Alzheimer's disease, measured clinically by Mini-Mental State Examination (MMSE) score, when the expression level of Apolipoprotein E4 protein spot N5302 is detected (N5302>0; (FIG. 19C) or not detected (N5302=0; FIG. 19A) in the blood serum. Severity of the Alzheimer's disease increases with decreasing MMSE score (Mild: MMSE=25-20; Moderate: MMSE=19-11; Severe: MMSE 510). A Box and Whisker graph (FIG. 19B) illustrates the comparative blood serum expression level (PPM) of Complement C3dg protein spot N1511 in age-matched control (AMC) subjects. Linear regression and Box and Whisker graphs were constructed using Analyze-it software for Microsoft Excel.

FIG. 20 is a summary diagram for the proposed functional relationships between the expression level of Complement protein biomarkers C3c1 protein spot N7310, C3c2a protein spot N9311, and C3dg protein spot N1511, Alzheimer's disease severity, and inflammatory response, when Apolipoprotein E4 N5302 protein was detected (N5302>0) or not detected (N5302=0) in blood serum of Alzheimer's disease patients. The diagram depicts the capacity for early detection of Alzheimer's disease, the measurement of disease severity and of the disease mechanism.

FIGS. 21A-21D depict statistical Dot, Box and Whiskers graphs (constructed using Analyze-it software for Microsoft Excel), illustrating the blood serum differential expression level (PPM) of Haptoglobin HP-1 proteins (FIG. 21A) spot N1514, (FIG. 21B) spot N2401, (FIG. 21C) Spot N2407 and (FIG. 21D) spot N3409, based on the quantitative 2D gel triplicate analysis data, obtained with samples from: 75 normal control individuals (Controls), 115 Alzheimer's disease patients (AD), 12 Parkinson's disease patients (PD), and 12 patients with AD-Like and Mixed disorders, including: Frontotemporal dementia (FTD), Frontotemporal dementia combined with Chronic Inflammatory Demyelinating polyneuropathy, Lewy body dementia (LBD), Vascular (Multi-infarct) dementia, Thalamic CVA combined with HX or Lung CA, Post-irradiation Encephalopathy, Seizures, Alcohol related dementia, Semantic dementia, Memory Dysfunction, neuro exam “Normal,” and Corticalbasal Ganglionic Degeneration (CBGD), with examples of the differences between the ranges of the patient and control groups and their statistical significance by analysis of variance (ANOVA-P) calculated by Analyze-it for Microsoft Excel with these data. E) Receiver Operator Characteristics (ROC) curve (constructed using Analyze-it software for Microsoft Excel), of biomarker N3409 when used as a single marker to distinguish between AD and PD patients.

FIG. 22A depicts a statistical Dot, Box and Whiskers graph (constructed using Analyze-it software for Microsoft Excel), illustrating the differential expression level (PPM) of the Total of Haptoglobin HP-1 proteins (spots N1514+N2401+N2407+N3409), in blood serum, based on the quantitative 2D gel triplicate analysis data obtained with blood serum samples from: 75 normal control individuals (Controls), 115 Alzheimer's disease patients (AD), 12 Parkinson's disease patients (PD), and 12 patients with AD-Like and Mixed disorders, including:

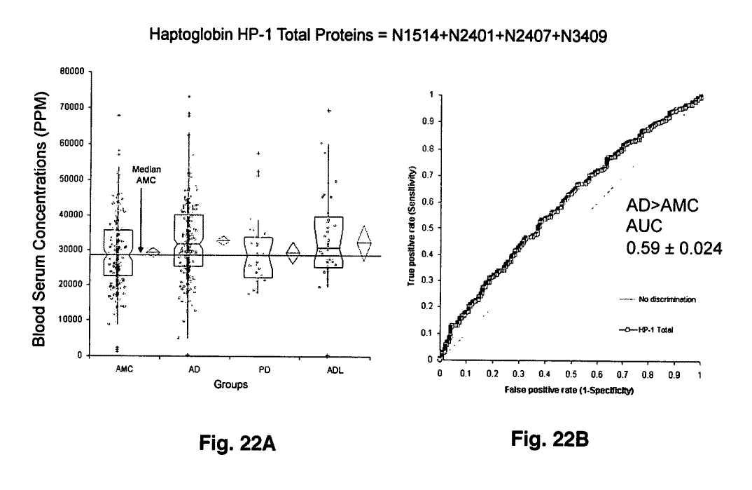

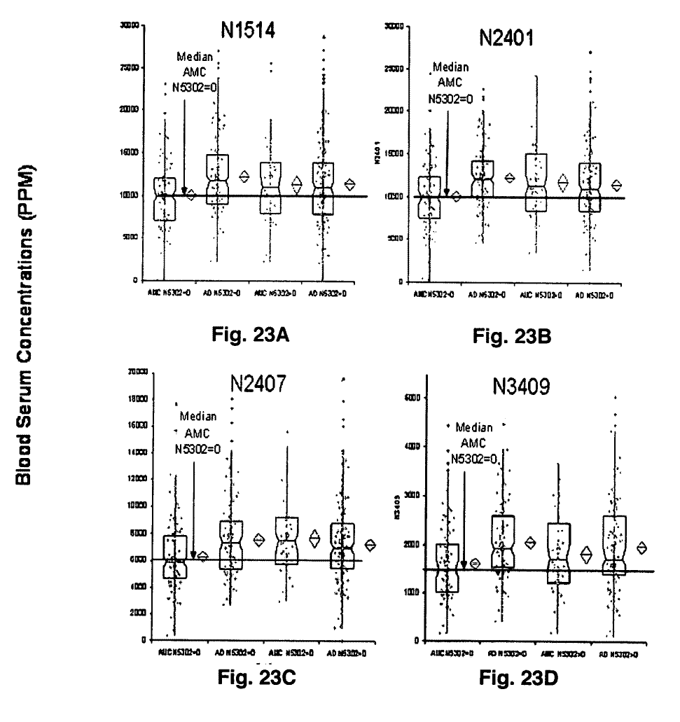

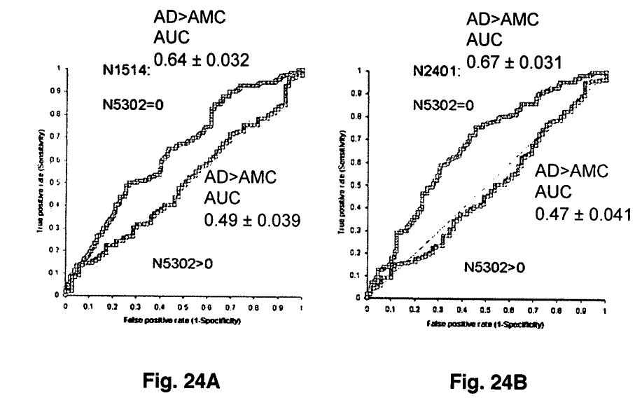

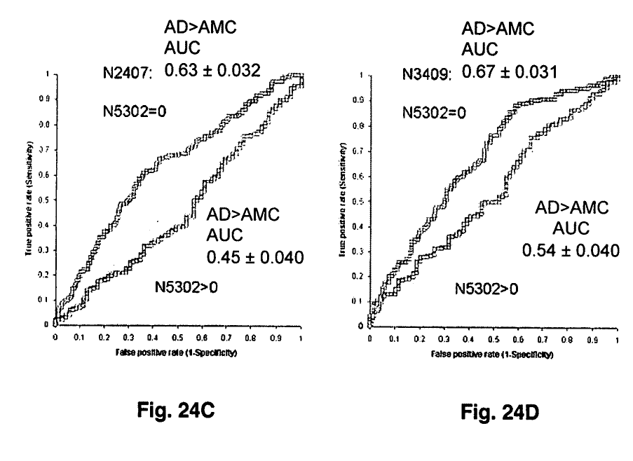

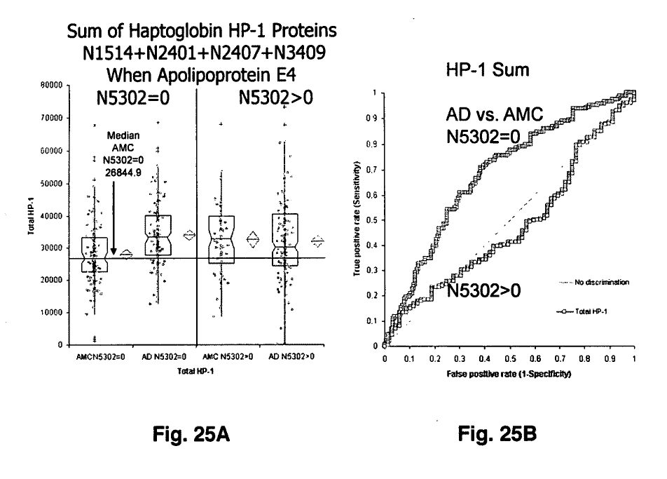

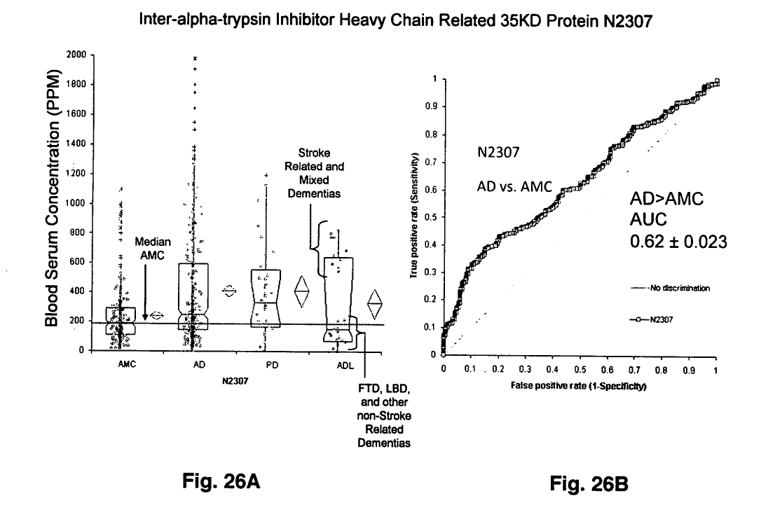

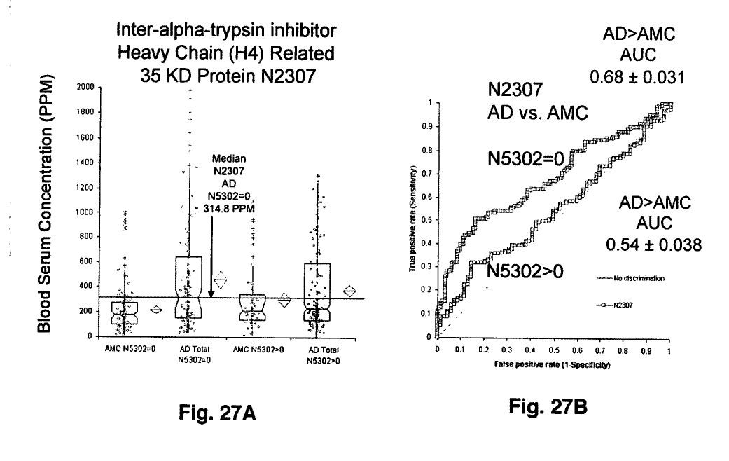

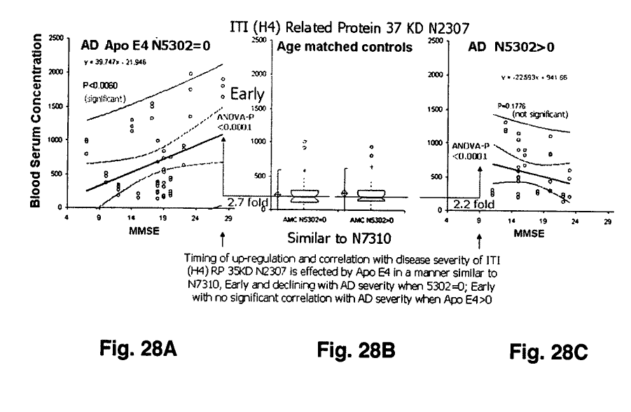

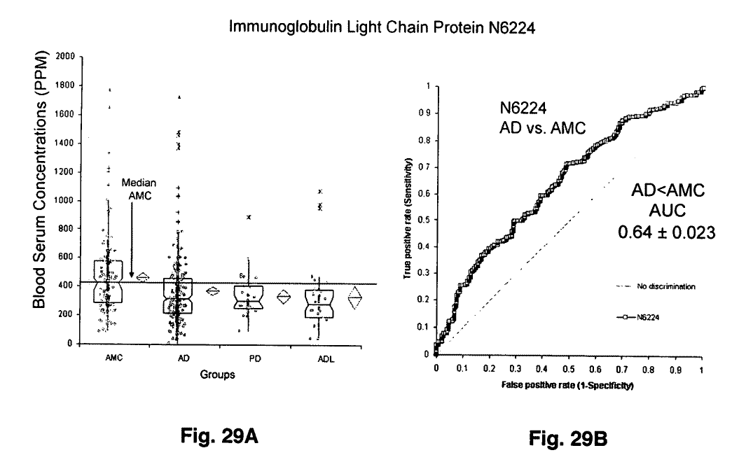

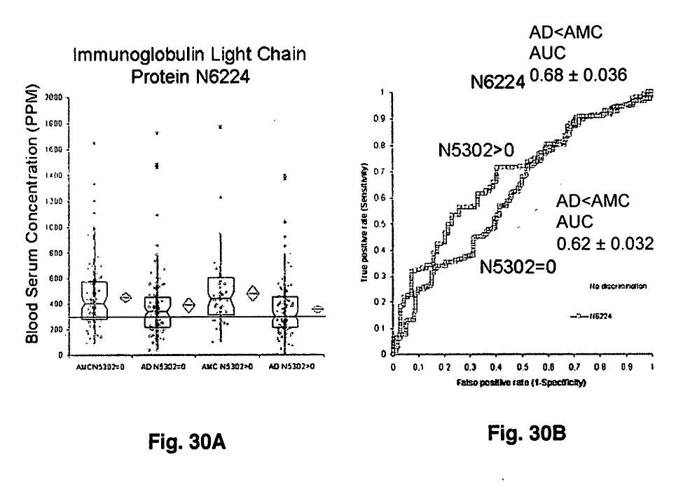

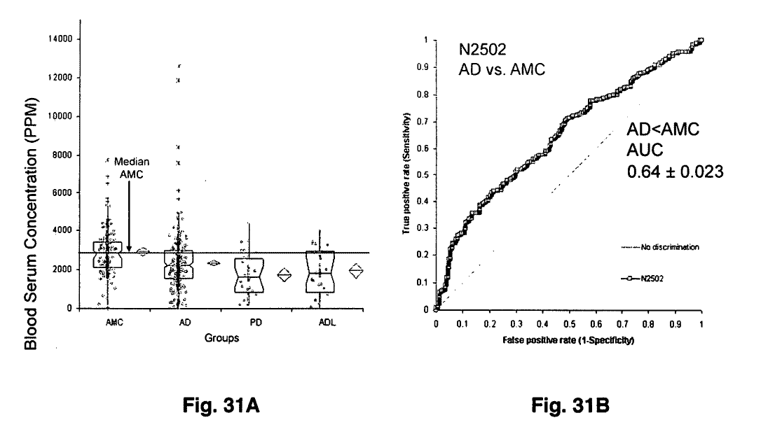

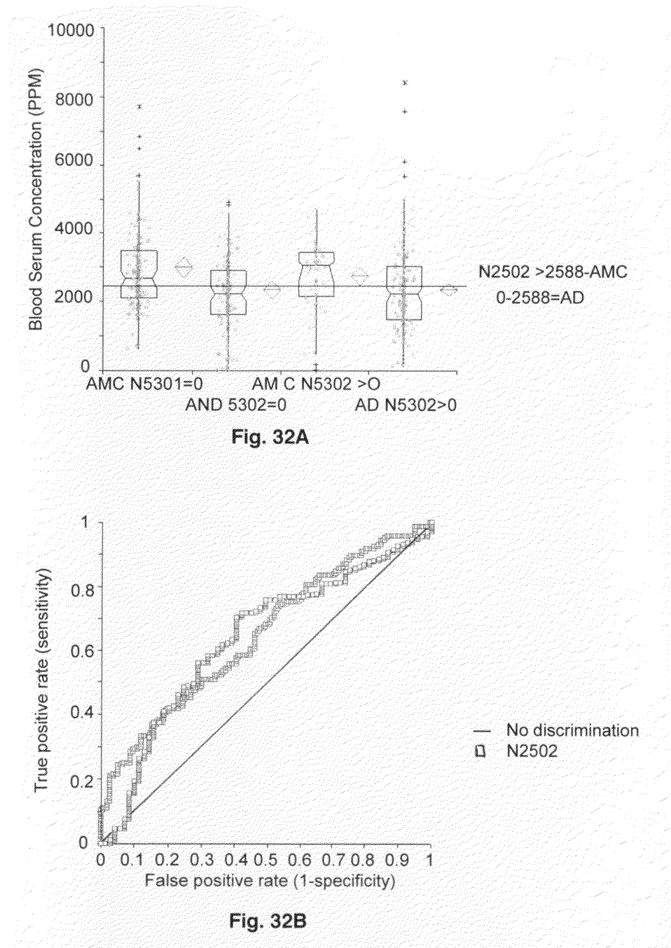

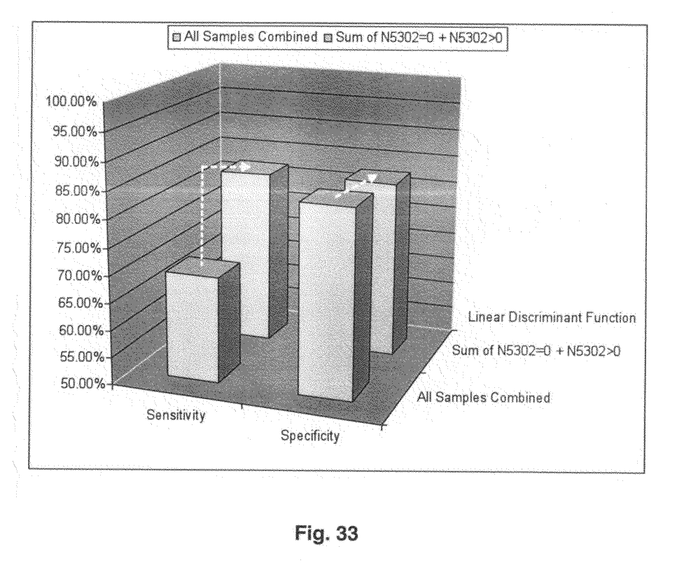

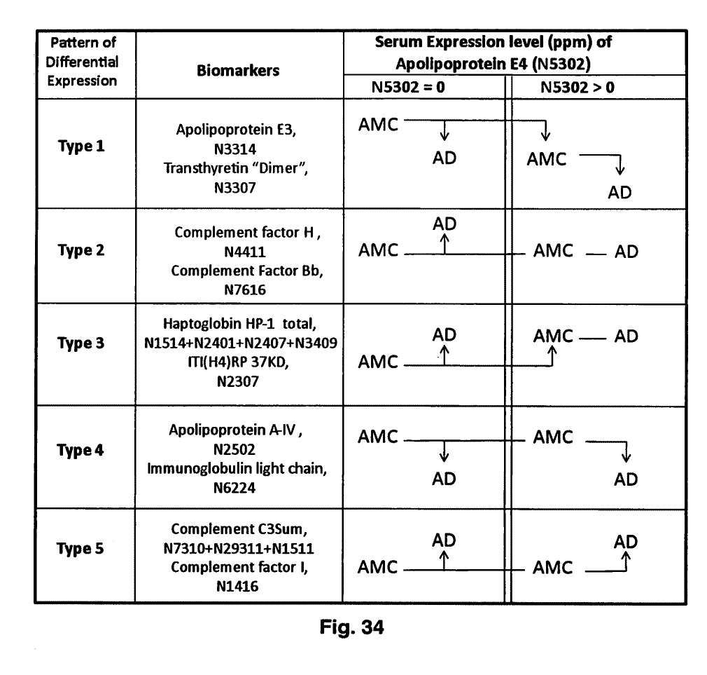

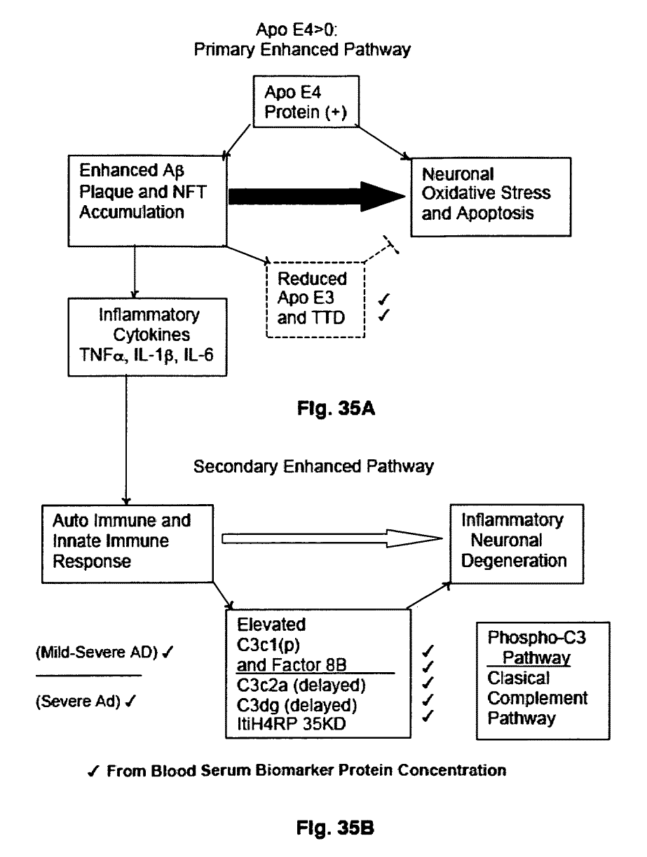

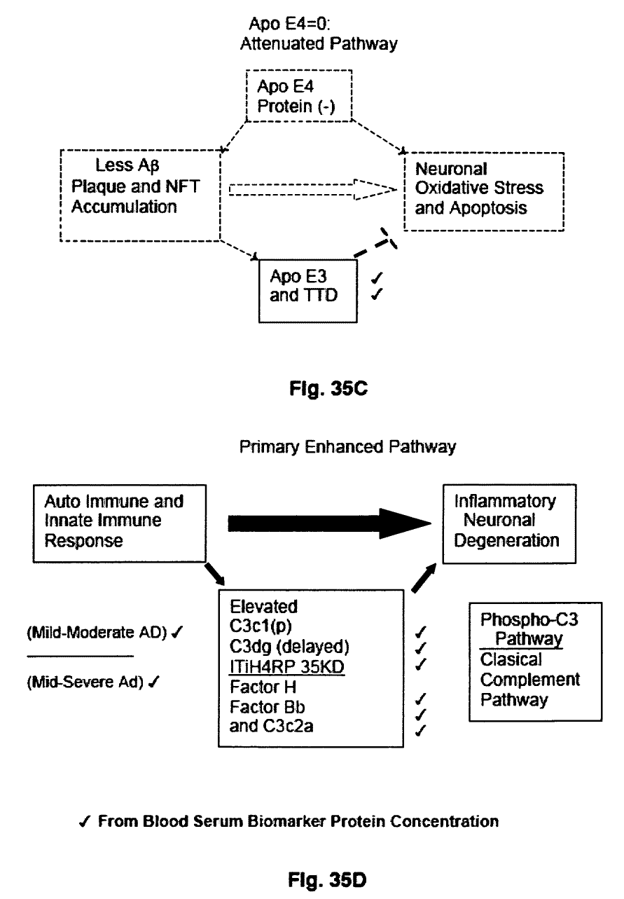

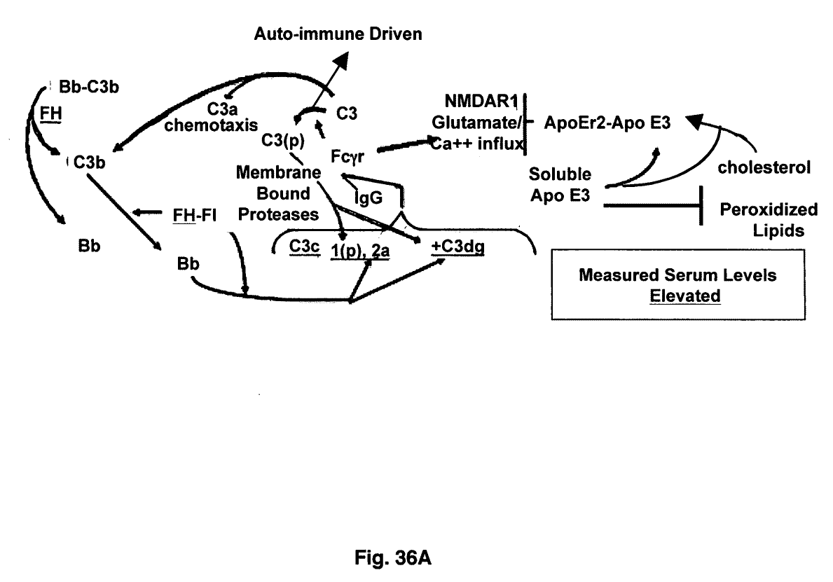

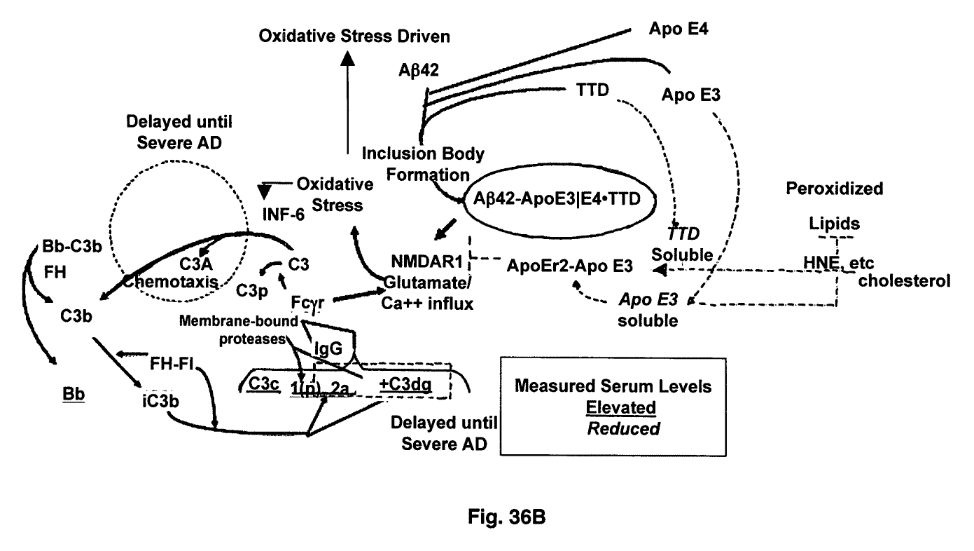

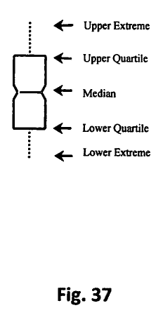

Frontotemporal dementia (FTD), Frontotemporal dementia combined with Chronic Inflammatory Demyelinating polyneuropathy, Lewy body dementia (LBD), Vascular (Multi-infarct) dementia, Thalamic CVA combined with HX or Lung CA, Post-irradiation Encephalopathy, Seizures, Alcohol related dementia, Semantic dementia, Memory Dysfunction, neuro exam “Normal,” and Corticalbasal Ganglionic Degeneration (CBGD), with examples of the differences between the ranges of the patient and control groups and their statistical significance by analysis of variance (ANOVA-P) and by Sensitivity, Specificity, and ROC probability (ROC-P), calculated by Analyze-it for Microsoft Excel with these data.
FIG. 22B is a Receiver Operator Characteristics (ROC) curve (constructed using Analyze-it software for Microsoft Excel) of Haptoglobin HP-1 protein spots N1514+N2401+N2407+N3409, when used to distinguish between AD patients and age-matched control (AMC) subjects with an area under the curve (AUC) of 0.59±0.024.

FIGS. 23A-23D depict statistical Dot, Box and Whiskers graphs (constructed using Analyze-it software for Microsoft Excel), illustrating the blood serum differential expression levels (PPM) of Haptoglobin HP-1 proteins: (FIG. 23A) spot N1514, (FIG. 23B) spot N2401, (FIG. 23C) Spot N2407, and (FIG. 23D) spot N3409, based on the quantitative 2D gel triplicate analysis data obtained with blood serum samples from patients with Alzheimer's disease (AD) and age matched normal controls as a function of whether Apolipoprotein E4 protein spot N5302 is detected (N5302>0) or not detected (N5302=0) in blood serum of the Alzheimer's disease (AD) patients and the age matched normal controls.

FIGS. 24A-24D depict the Receiver Operator Characteristics (ROC) curve (constructed using Analyze-it software for Microsoft Excel) of Haptoglobin HP-1 proteins: FIG. 24A) spot N1514, FIG. 24B) spot N2401, FIG. 24C) Spot N2407, and FIG. 24D) spot N3409, when used separately to distinguish between patients with Alzheimer's disease (AD) and age matched normal controls (AMC) as a function of whether Apolipoprotein E4 protein (spot N5302) is detected (N5302>0) or not detected (N5302=0) in blood serum of the Alzheimer's disease (AD) patients and the age matched normal controls.

FIG. 25A depicts a statistical Dot, Box and Whiskers graph (constructed using Analyze-it software for Microsoft Excel), illustrating the blood serum differential expression level (PPM) of the Total of Haptoglobin HP-1 proteins (spots N1514+N2401+N2407+N3409) as a function of whether Apolipoprotein E4 protein (spot N5302) is detected (N5302>0) or not detected (N5302=0) in blood serum of the Alzheimer's disease (AD) patients and the age matched normal controls. FIG. 25B is a Receiver Operator Characteristics (ROC) curve (constructed using Analyze-it software for Microsoft Excel) of Haptoglobin HP-1 protein total spots (N1514+N2401+N2407+3409) when used to distinguish between AD patients and age-matched control (AMC) subjects as a function of whether Apolipoprotein E4 protein spot N5302 is detected (N5302>0) or not detected (N5302=0) in blood serum of the Alzheimer's disease (AD) patients and the age matched normal controls.

FIG. 26A is a Box and Whiskers graph (constructed using Analyze-it software for Microsoft Excel), illustrating the blood serum differential expression level (PPM) of Inter-alpha-trypsin Inhibitor Heavy Chain (H4) related 35 KD protein (spot N2307), based on the quantitative 2D gel triplicate analysis data obtained with blood serum samples from: 75 normal control individuals (Controls), 115 Alzheimer's disease patients (AD), 12 Parkinson's disease patients (PD), and 12 patients with AD-Like and Mixed disorders, including: Frontotemporal dementia (FTD), Frontotemporal dementia combined with Chronic Inflammatory Demyelinating polyneuropathy, Lewy body dementia (LBD), Vascular (Multi-infarct) dementia, Thalamic CVA combined with HX or Lung CA, Post-irradiation Encephalopathy, Seizures, Alcohol related dementia, Semantic dementia, Memory Dysfunction, neuro exam “Normal,” and Corticalbasal Ganglionic Degeneration (CBGD), with examples of the differences between the ranges of the patient and control groups and their statistical significance by analysis of variance (ANOVA-P) and by Sensitivity, Specificity, and ROC probability (ROC-P), calculated by Analyze-it for Microsoft Excel with these data. FIG. 26B is a Receiver Operator Characteristics (ROC) curve (constructed using Analyze-it software for Microsoft Excel) of Inter-alpha-trypsin Inhibitor Heavy Chain (H4) related 35 KD protein (spot N2307) when used to distinguish between AD patients and age-matched control (AMC) subjects with an area under the curve (AUC) of 0.62±0.023.

FIG. 27A is a statistical Box and Whiskers graph illustrating the blood serum differential expression level (PPM) of Inter-alpha-trypsin Inhibitor Heavy Chain (H4) related 35 KD protein (spot N2307) as a function of whether Apolipoprotein E4 protein (spot N5302) is detected (N5302>0) or not detected (N5302=0) in blood serum of the Alzheimer's disease (AD) patients and the age matched normal controls. FIG. 27B is a Receiver Operator Characteristics (ROC) curve (constructed using Analyze-it software for Microsoft Excel) of Inter-alpha-trypsin Inhibitor Heavy Chain (H4) related 35 KD protein (spot N2307) as a function of whether Apolipoprotein E4 protein spot N5302 when used to distinguish between AD patients and age-matched control (AMC) subjects as a function of whether Apolipoprotein E4 protein spot N5302 is detected (N5302>0) or not detected (N5302=0) in blood serum of the Alzheimer's disease (AD) patients and the age matched normal controls.

FIGS. 28A-28C are statistical linear regression correlation of the blood serum expression level (PPM) of Inter-alpha-trypsin Inhibitor Heavy Chain (H4) related 35 KD protein spot N2307, with the severity of the Alzheimer's disease, measured clinically by Mini-Mental State Examination (MMSE) score, when the expression level of Apolipoprotein E4 protein spot N5302 is detected (N5302>0; FIG. 28C) or not detected (N5302=0; FIG. 28A) in the blood serum. Severity of the Alzheimer's disease increases with decreasing MMSE score (Mild: MMSE=25-20; Moderate: MMSE=19-11; Severe: MMSE≦10). Box and Whisker graph (FIG. 28B) illustrates the comparative blood serum expression level (PPM) of Inter-alpha-trypsin Inhibitor Heavy Chain (H4) related 35 KD protein spot N2307 in age-matched control (AMC) subjects. Linear regression and box and Whisker graphs were constructed using Analyze-it software for Microsoft Excel.

FIG. 29A is a statistical Box and Whiskers graph (constructed using Analyze-it software for Microsoft Excel), illustrating the blood serum differential expression level (PPM) of Immunoglobulin Light Chain Protein spot N6224, based on the quantitative 2D gel triplicate analysis data obtained with blood serum samples from: 75 normal control individuals (Controls), 115 Alzheimer's disease patients (AD), 12 Parkinson's disease patients (PD), and 12 patients with AD-Like and Mixed disorders, including: Frontotemporal dementia (FTD), Frontotemporal dementia combined with Chronic Inflammatory Demyelinating polyneuropathy, Lewy body dementia (LBD), Vascular (Multi-infarct) dementia, Thalamic CVA combined with HX or Lung CA, Post-irradiation Encephalopathy, Seizures, Alcohol related dementia, Semantic dementia, Memory Dysfunction, neuro exam “Normal,” and Corticalbasal Ganglionic Degeneration (CBGD), with examples of the differences between the ranges of the patient and control groups and their statistical significance by analysis of variance (ANOVA-P) and by Sensitivity, Specificity, and ROC probability (ROC-P), calculated by Analyze-it for Microsoft Excel with these data. FIG. 29B is a Receiver Operator Characteristics (ROC) curve (constructed using Analyze-it software for Microsoft Excel) of Immunoglobulin Light Chain Protein (spot N6224) when used to distinguish between AD patients and age-matched control (AMC) subjects with an area under the curve (AUC) of 0.64±0.023.

FIG. 30A is a statistical Box and Whiskers graph (constructed using Analyze-it software for Microsoft Excel), illustrating the blood serum differential expression level (PPM) of Immunoglobulin Light Chain Protein spot N6224 as a function of whether Apolipoprotein E4 protein spot N5302 is detected (N5302>0) or not detected (N5302=0) in blood serum of the Alzheimer's disease (AD) patients and the age matched normal controls. FIG. 30B is a Receiver Operator Characteristics (ROC) curve (constructed using Analyze-it software for Microsoft Excel) of Immunoglobulin Light Chain Protein spot N6224 as a function of whether Apolipoprotein E4 protein (spot N5302) when used to distinguish between AD patients and age-matched control (AMC) subjects as a function of whether Apolipoprotein E4 protein (spot N5302) is detected (N5302>0) or not detected (N5302=0) in blood serum.

FIG. 31A is a statistical Box and Whiskers graph (constructed using Analyze-it software for Microsoft Excel), illustrating the blood serum differential expression level (PPM) of Apolipoprotein A-IV Protein spot N2502, based on the quantitative 2D gel triplicate analysis data obtained with blood serum samples from: 75 normal control individuals (Controls), 115 Alzheimer's disease patients (AD), 12 Parkinson's disease patients (PD), and 12 patients with AD-Like and Mixed disorders, including: Frontotemporal dementia (FTD), Frontotemporal dementia combined with Chronic Inflammatory Demyelinating polyneuropathy, Lewy body dementia (LBD), Vascular (Multi-infarct) dementia, Thalamic CVA combined with HX or Lung CA, Post-irradiation Encephalopathy, Seizures, Alcohol related dementia, Semantic dementia, Memory Dysfunction, neuro exam “Normal,” and Corticalbasal Ganglionic Degeneration (CBGD), with examples of the differences between the ranges of the patient and control groups and their statistical significance by analysis of variance (ANOVA-P) and by Sensitivity, Specificity, and ROC probability (ROC-P), calculated by Analyze-it for Microsoft Excel with these data. FIG. 32B is a Receiver Operator Characteristics (ROC) curve (constructed using Analyze-it software for Microsoft Excel) of Apolipoprotein A-IV Protein (spot N2502) when used to distinguish between AD patients and age-matched control (AMC) subjects with an area under the curve (AUC) of 0.64±0.023.

FIG. 32A is a statistical Box and Whiskers graph illustrating the blood serum differential expression level (PPM) of Apolipoprotein A-IV Protein spot N2502 as a function of whether Apolipoprotein E4 protein spot N5302 is detected (N5302>0) or not detected (N5302=0) in blood serum of the Alzheimer's disease (AD) patients and the age matched normal controls. FIG. 32B is a Receiver Operator Characteristics (ROC) curve (constructed using Analyze-it software for Microsoft Excel) of Apolipoprotein A-IV Protein (pot N2502 as a function of whether Apolipoprotein E4 protein spot N5302, when used to distinguish between AD patients and age-matched control (AMC) subjects as a function of whether Apolipoprotein E4 protein spot N5302 is detected (N5302>0) or not detected (N5302=0) in blood serum.

FIG. 33 illustrates the enhanced sensitivity obtained using the blood serum concentrations of protein biomarkers. The approach employs the separation of Alzheimer's disease patients and age-matched control subjects into two categories, based on the detection or lack of detection of Apolipoprotein E4. N5302 in their blood serum. A multivariate biostatistical analysis is applied to each of the 2 groups, employing all the biomarkers (N3314, N3317, N4411, N7616, HP-1 total [N1514+N2401+N2407+N3409], N7310, N9311, N1511, N2307, N2502, and N6224), followed by summing the separate results of the 2 multivariate biostatistical analyses of the sorted categories. As shown, this approach provides substantial improvement in diagnostic capability (sensitivity increased from 69.6% to 82.3%) over the non-sorted approach, which includes combining all the biomarkers and all the samples into a single multivariate biostatistical analysis.

FIG. 34 illustrates the 5 types of differences in the differential expression of the protein biomarkers in the blood serum of the sorted Alzheimer's disease patients in relation to the measured concentrations of Apolipoprotein E4 protein spot N5302, when it is detected (N5302>0) and not detected (N5302=0) in the blood. These differences form the basis for the improvements of sensitivity of diagnosis of Alzheimer's disease illustrated in FIG. 33. In type 1, the serum expression level (PPM) of biomarkers Apolipoprotein E3 protein spot N3314 and Transthyretin dimer protein spot N3307 in Alzheimer's disease patients are lower than age-matched control (AMC) subjects, when Apolipoprotein E4 protein spot N5302 is detected (N5302>0) or not detected (N5302=0) in serum. In type 2, the serum expression level (PPM) of biomarkers Complement Factor H protein spot N4411 and Complement Factor Bb protein spot N7616 in Alzheimer's disease patients are higher than age-matched control (AMC) subjects, when Apolipoprotein E4 protein spot N5302 is not detected (N5302=0), while equal to the serum expression levels of AMC, when N5302 is detected (N5302>0) in serum. In type 3, the serum expression level (PPM) of biomarkers Haptoglobin HP-1 total protein spots N1514+N2401+N2407+N3409 and ITI (H4) RP 37 KD protein spot N2307 in Alzheimer'patients are higher than age-matched control (AMC) subjects, when Apolipoprotein E4 protein spot N5302 is not detected (N5302=0) and equal to serum expression levels of AMC subjects, when N5302 is detected (N5302>0) in serum. In type 4, the serum expression level (PPM) of biomarkers Apolipoprotein A-IV protein spot N2502 and Immunoglobulin light chain protein spot N6224 in Alzheimer's disease patients are lower than age-matched control (AMC) subjects, when Apolipoprotein E4 protein spot N5302 is detected (N5302>0) and not detected (N5302=0) in serum. In type 5, the serum expression level (PPM) of biomarkers Complement C3csum protein spots N7310+N9311+N1511 and Complement Factor I protein spot N 1416 in Alzheimer's disease patients are higher than age-matched control (AMC) subjects, when Apolipoprotein E4 protein spot N5302 is detected (N5302>0) and not detected (N5302=0) in serum

FIG. 35 illustrates the differences in the disease pathways of neuronal degeneration, and which predominate or are attenuated, based on the differences in the differential expression of the protein biomarkers in the blood serum of the sorted Alzheimer's disease patients as illustrated in FIG. 34. In patients with Alzheimer's disease, when the serum expression level of Apolipoprotein E4 protein spot N5302 is detected (N5302>0, A and B), the elevated level of this biomarker is associated with A) markedly reduced serum expression of Apolipoprotein E3 protein spot N3314 and Transthyretin “Dimer” protein spot N3307, (FIG. 34, Type 1), enhanced A_plaque and accumulation of Neurofibrillary tangles (NET), and elevated inflammatory cytokines in blood. These changes lead to neuronal oxidative stress and apoptosis and also initiate B) secondary immune and innate inflammatory responses that enhance neuronal degeneration, associated with increased serum levels of phosphorylated C3c1 protein spot N7310, Factor Bb protein spot N7616, non-phosphorylated Complement C3c2a protein spot N9311, C3dg protein spot N1511, and ITI(H4)RP. In patients with Alzheimer's disease, when the serum expression level of Apolipoprotein E4 protein spot N5302 is not detected (N5302=0, C and D), the non-detected level of N5302 is associated with slightly decreased serum expression of Apolipoprotein E3 protein spot N3314 and Transthyretin “Dimer” protein spot N3307. The close to normal levels of these 2 biomarkers are associated with neuronal protection. However, these Alzheimer's patients showed elevated serum level of phosphorylated Complement C3c1 protein spot N7310, Factor Bb protein spot N7616, Factor H protein spot N4411, non-phosphorylated Complement C3c2a protein spot N9311, and Complement C3dg protein spot N1511, and ITI(H4)RP protein spot N2307. These biomarkers are associated with autoimmune and innate inflammatory responses, which lead to neuronal degeneration.

FIGS. 36A (auto-immune driven) and 36B (oxidative stress driven) illustrate the differences in Alzheimer's disease biochemical mechanisms of neuronal degeneration, and whether they predominate or are attenuated, based on the identities, the biochemical roles of the protein biomarkers, and the differences in the disease pathways illustrated in FIG. 35.

FIG. 37 shows a visual representation of the statistical confidence levels.

Table 1 depicts the reproducibility of quantitation in 2D gels whereby 9 replicate analyses were performed with an individual sample of bovine serum albumin standard, where the sample was separated by 2D gel electrophoresis into a characteristic set of 5 spots which were then subjected to quantitation. The raw density counts (Gaussian Peak Values) are shown as are the individual values, averages, standard deviations, % Coefficients of Variation, and the quantity of the protein in nanograms (ng) for each spot.

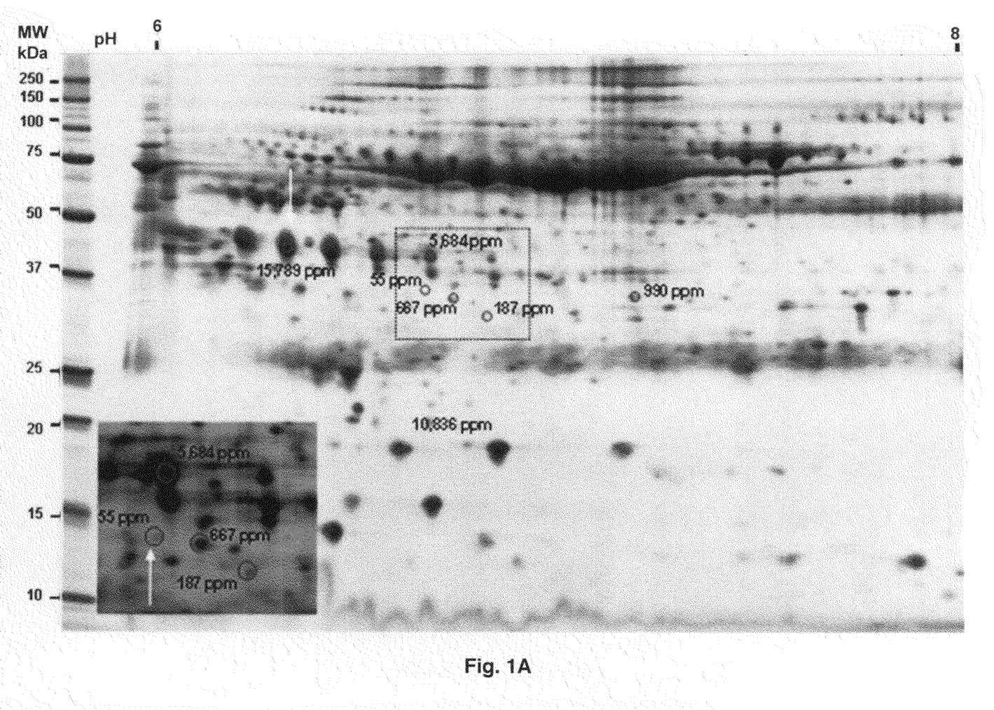

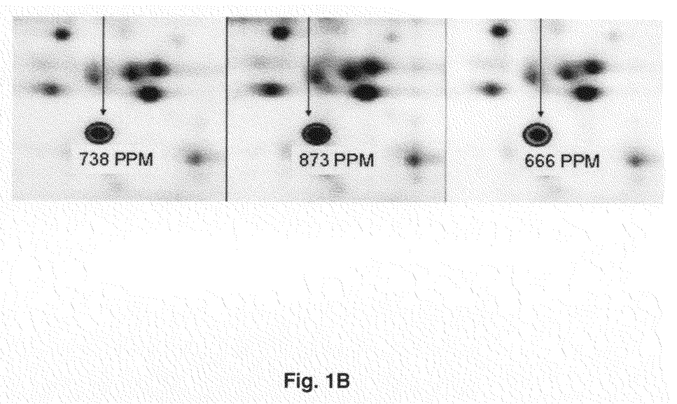

Table 2 illustrates the reproducibility of quantitation of protein spots over the dynamic range of the 2D gel assay of human serum depicted in FIG. 1A. Shown are replicate (14×) 2D gel analyses each of the quantitation of 13 different protein spots ranging from 13,542 ppm to 72 ppm with a coefficient of variation of ≦20% (n=14) where 72 ppm is approximately 10 fold higher than the limit of detection (LOD=5-10 ppm) of the assay.

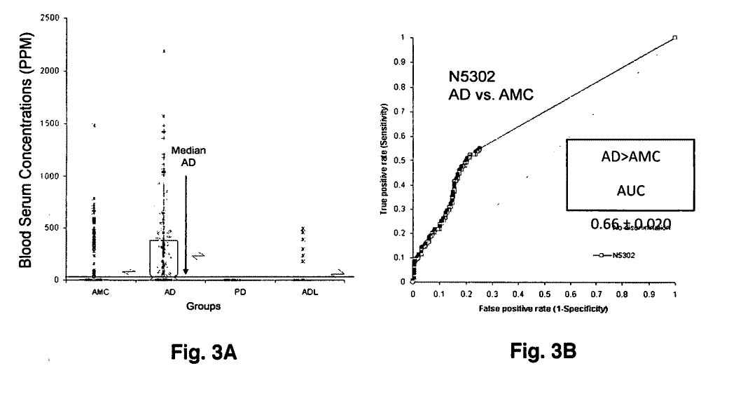

Table 3 illustrates the summary statistics for the graph depicted in FIG. 3.

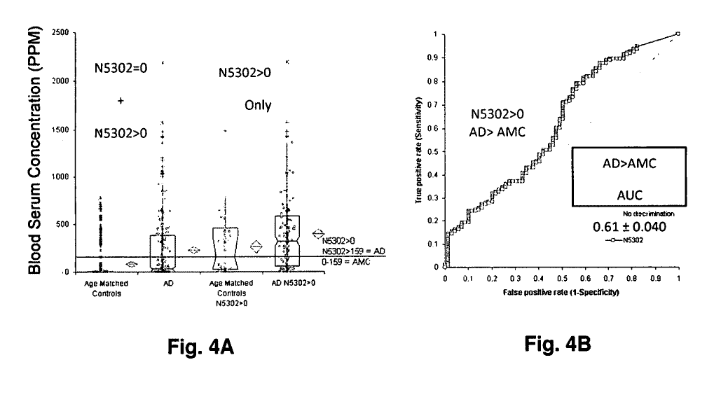

Table 4 illustrates the summary statistics for the graph depicted in FIG. 4.

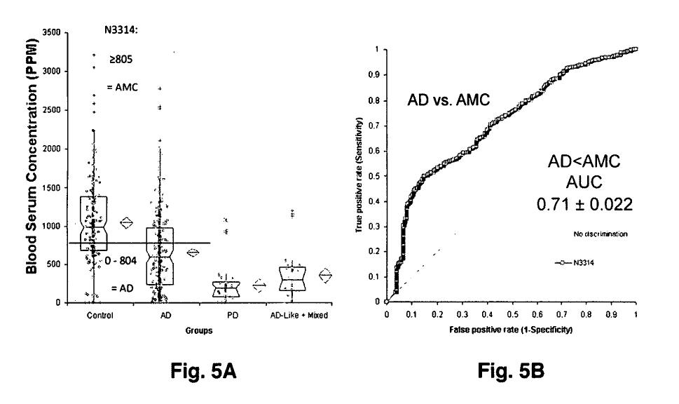

Table 5 illustrates the summary statistics for the graph depicted in FIG. 5.

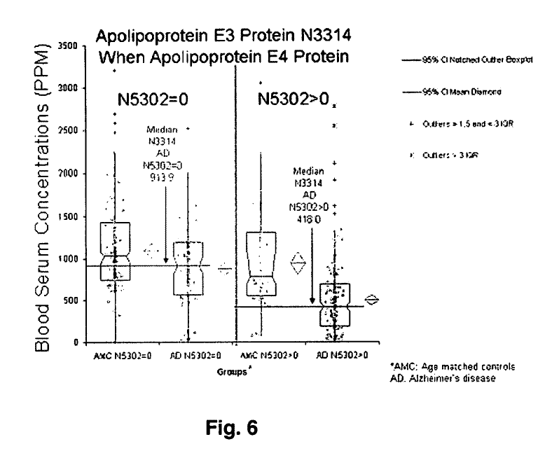

Table 6 illustrates the summary statistics for the graph depicted in FIG. 6.

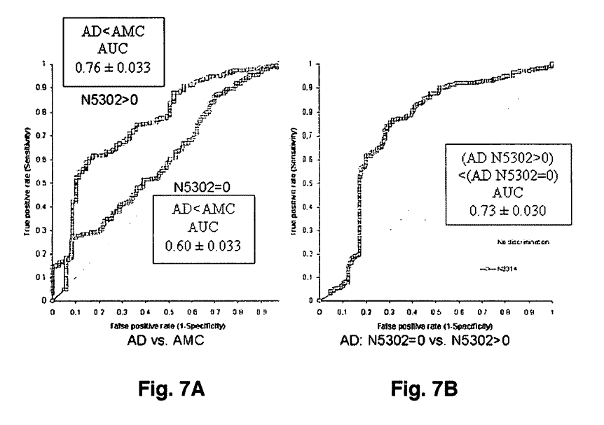

Table 7 illustrates the summary statistics for the graph depicted in FIG. 7.

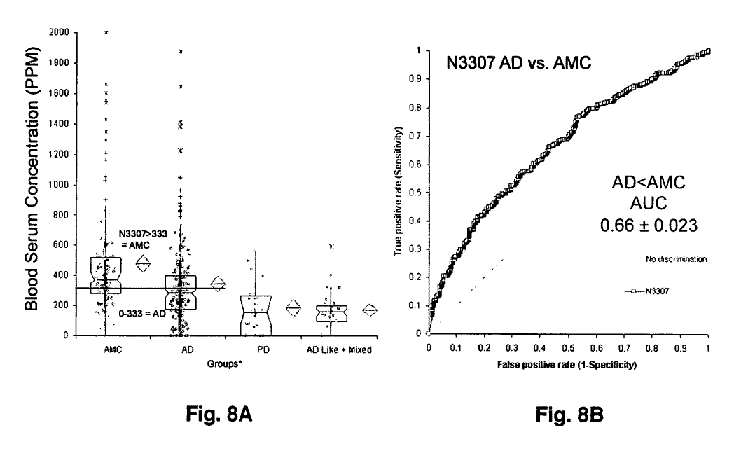

Table 8 illustrates the summary statistics for the graphs depicted in FIG. 8.

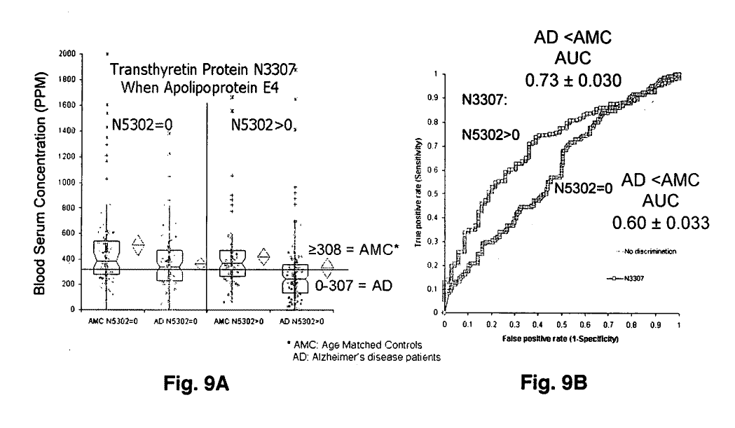

Table 9 illustrates the summary statistics for the graphs depicted in FIG. 9.

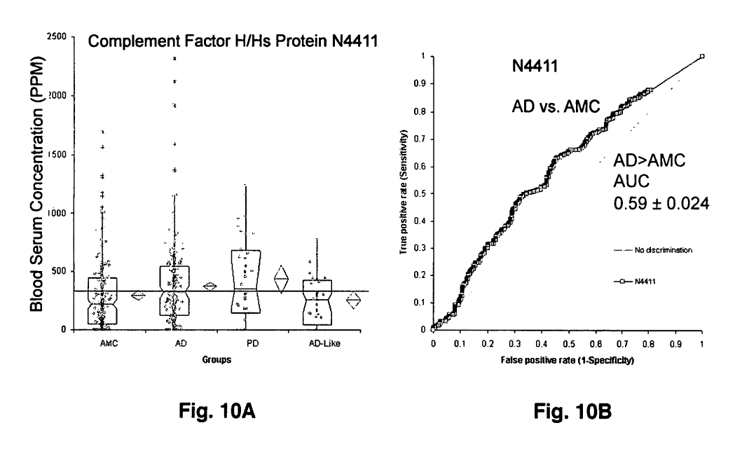

Table 10 illustrates the summary statistics for the graphs depicted in FIG. 10.

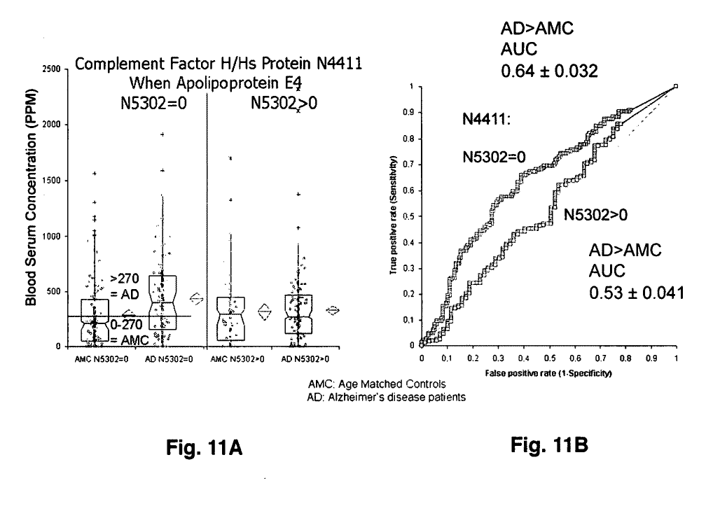

Table 11 illustrates the summary statistics for the graph depicted in FIG. 11.

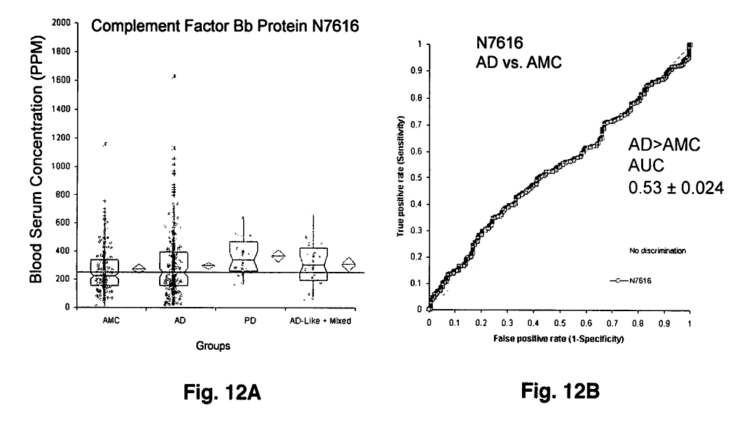

Table 12 illustrates the summary statistics for the graph depicted in FIG. 12.

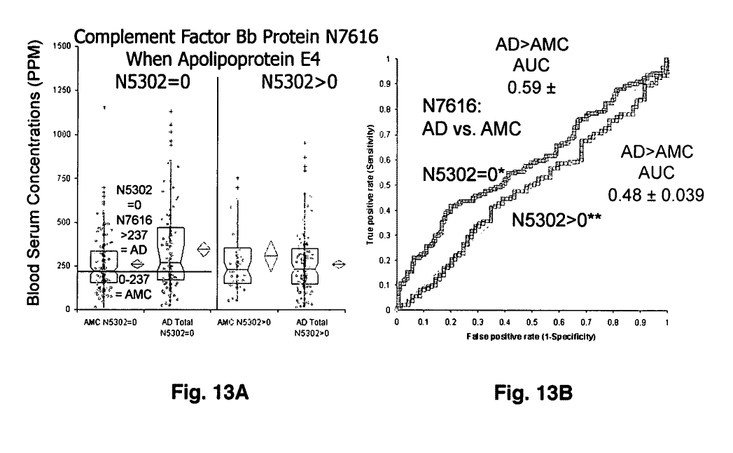

Table 13 illustrates the summary statistics for the graph depicted in FIG. 13.

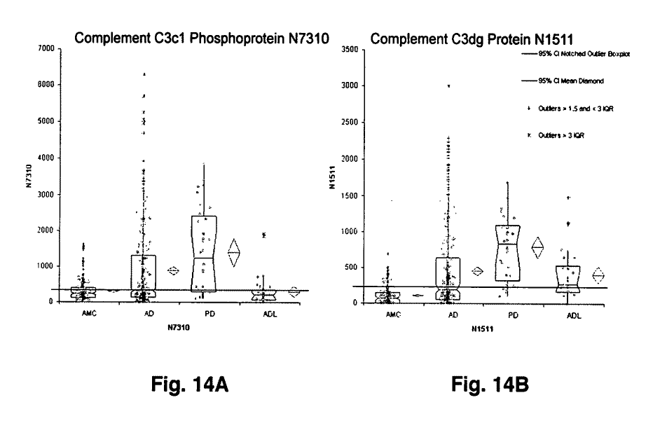

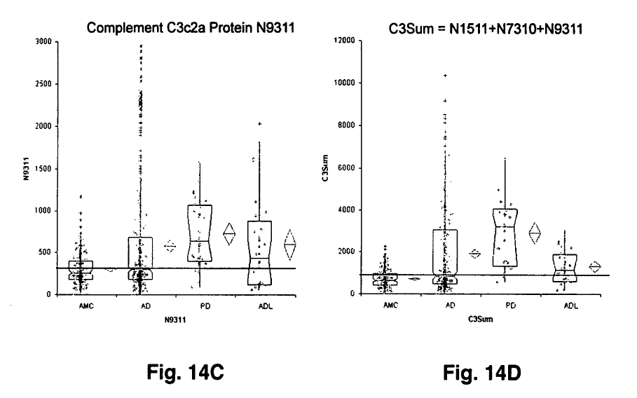

Table 14 illustrates the summary statistics for the graph depicted in FIG. 14.

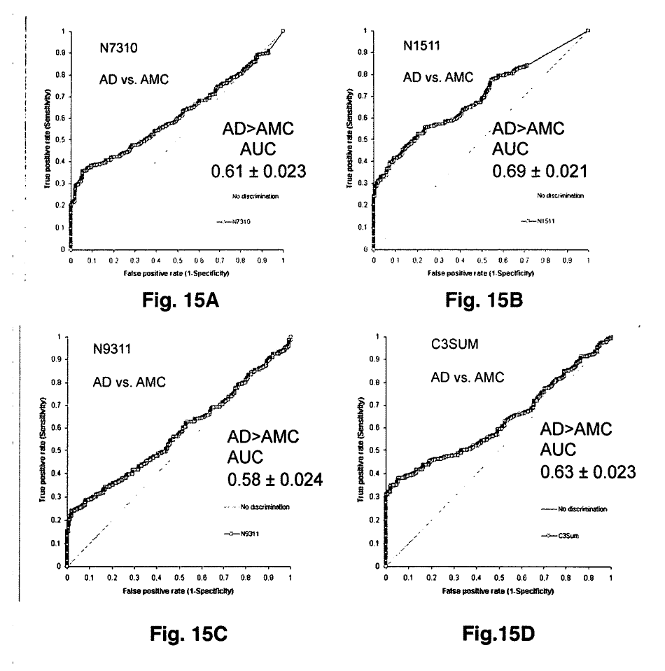

Table 15 illustrates the summary statistics for the graphs depicted in FIG. 15.

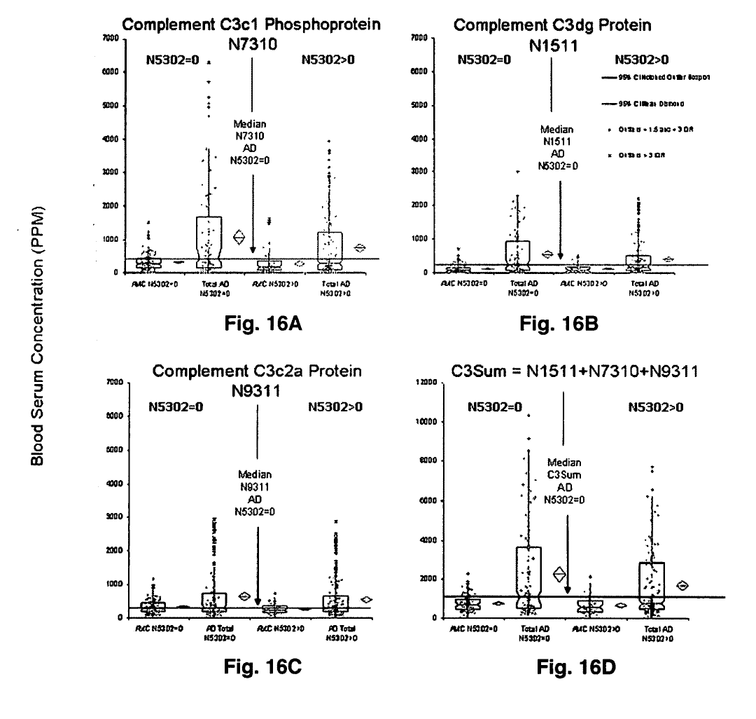

Table 16 illustrates the summary statistics for the graphs depicted in FIG. 16.

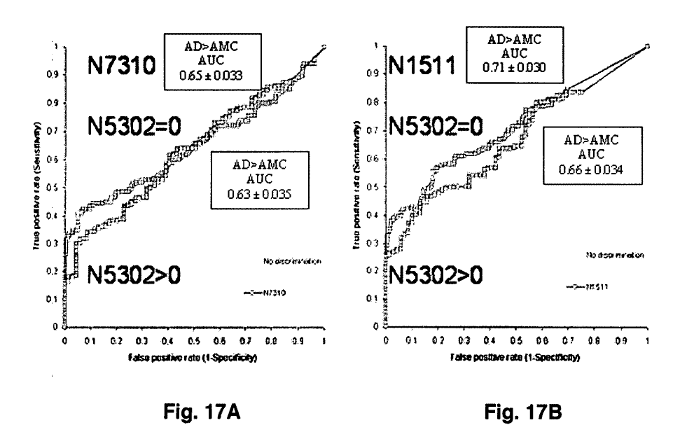

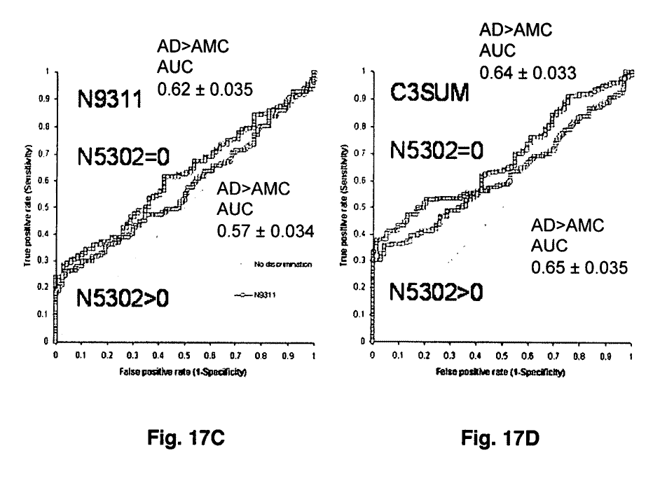

Table 17 illustrates the summary statistics for the graph depicted in FIG. 17.

Table 18 illustrates the summary statistics for the graph depicted in FIG. 22.

Table 19 illustrates the summary statistics for the graphs depicted in FIG. 25.

Table 20 illustrates the summary statistics for the graph depicted in FIG. 26.

Table 21 illustrates the summary statistics for the graph depicted in FIG. 27.

Table 22 illustrates the summary statistics for the graph depicted in FIG. 29.

Table 23 illustrates the summary statistics for the graph depicted in FIG. 30.

Table 24 illustrates the summary statistics for the graph depicted in FIG. 31.

Table 25 illustrates the summary statistics for the graphs depicted in FIG. 32.

Table 26: illustrates the summary statistics of multivariate linear discriminant analysis (constructed using SAS software) for the graph in FIG. 33.

Table 27 illustrates the different disease mechanisms of familial and sporadic neurodegenerative diseases revealed by the patients' blood serum biomarkers

Table 28 illustrates the different disease mechanisms of PD and ALS neuronal degeneration revealed by patients' blood serum biomarkers.

Table 29 illustrates the general applications of the invention.

SEQ ID NO. 1 illustrates the identification of the amino acid sequence of the Apolipoprotein E4 protein precursor of protein spot N5203 wherein amino acids 1-17 are the signal peptide or leader sequence which is removed to make the mature protein.

SEQ ID NO. 2 illustrates the identification of the amino acid sequence of protein spot N5302 as the full size mature Apolipoprotein E4 protein after trimming the signal peptide off the amino terminal end of the molecule.

SEQ ID NO. 3 illustrates the identification of the amino acid sequence of the Apolipoprotein E3 protein precursor of protein spot N3314 wherein amino acids 1-17 are the signal peptide or leader sequence which is removed to make the mature protein.

SEQ ID NO. 4 illustrates the identification of the amino acid sequence of protein spot N3314 as the full size mature Apolipoprotein E3 protein after trimming the signal peptide off the amino terminal end of the molecule.

SEQ ID NO. 5 illustrates the identification of the amino acid sequence of Transthyretin “Dimer” Protein spot N3307, whose molecular weight by 2D gel electrophoresis is twice that of the molecular weight estimated using the amino acid sequence.

SEQ ID NO. 6 illustrates the identification of the amino acid sequence of Complement C3, the parent precursor protein of Complement C3c1 protein spot N7310 (Tyrosine Phosphorylated, amino acids 749-951); C3c2a protein spot N9311 (not tyrosine phosphorylated, amino acids 749-951); and C3dg protein spot N1511 (amino acids 955-1303).

SEQ ID NO. 7 illustrates the identification of the amino acid sequence of tyrosine phosphorylated Complement C3c1 Protein spot N7310, derived from the tyrosine phosphorylated variant of Complement C3 (SEQ ID NO. 6, amino acids 749-951).

SEQ ID NO. 8 (identical to SEQ ID NO. 7 but not tyrosine phosphorylated) illustrates the identification of the amino acid sequence of Complement C3c2a protein spot N9311, derived from the non tyrosine phosphorylated variant of Complement C3 (SEQ ID NO. 6, amino acids 749-951).

SEQ ID NO. 9 illustrates the identification of the amino acid sequence of Similar to C3, alternative parent precursor for an alternative C3dg isoform of protein spot N1511 (amino acids 902-1256), but not for C3c1 protein spot N7310 nor for C3c2a protein spot N9311.

SEQ ID NO. 10 illustrates the identification of the amino acid sequence of Complement C3dg protein spot N1511, derived from Complement C3 (SEQ ID NO. 6, amino acids 955-1303).

SEQ ID NO. 11 illustrates the identification of the amino acid sequence of Complement C3dg alternate isoform for protein spot N1511, derived from Similar to C3 (amino acid SEQ ID NO. 9; amino acids 902-1256).

SEQ ID NO. 12 illustrates the identification of the amino acid sequence of Complement Factor Bb protein spot N7616.

SEQ ID NO. 13 illustrates the identification of the amino acid sequence of Complement Factor H Parent Protein precursor of Complement Factor H/Hs protein spot N4411.

SEQ ID NO. 14 illustrates the identification of the amino acid sequence of Complement Factor Hs (Short Splice Form) alternate parent of Complement Factor H/Hs protein spot N4411.

SEQ ID NO. 15 illustrates the amino acid sequence of Complement Factor H/Hs protein spot N4411, derived from either SEQ ID NO. 13 and/or SEQ ID NO. 14.

SEQ ID NO. 16 illustrates the identification of the amino acid sequence of Inter alpha trypsin inhibitor heavy (H4) chain related protein, parent of the 35 KD protein spot N2307.

SEQ ID NO. 17 illustrates the identification of the amino acid sequence of Inter alpha trypsin inhibitor heavy (H4) chain related 35 KD protein isoform 1, protein spot N2307.

SEQ ID NO. 18 illustrates the identification of the amino acid sequence of Inter alpha trypsin inhibitor heavy (H4) chain related protein 35 KD isoform 2, alternate protein of spot N2307.

SEQ ID NO. 19 illustrates the identification of the amino acid sequence of Haptoglobin HP-1 Protein spots N1514; N2401; N2407; N3409.

SEQ ID NO. 20 illustrates the identification of the amino acid sequence of Complement Factor I Protein spot N1416.

SEQ ID NO. 21 illustrates the identification of the amino acid sequence of Immunoglobulin Light Chain Protein spot N6224.

SEQ ID NO. 22 illustrates the identification of the amino acid sequence of Apolipoprotein A-IV Protein spot N2502.

### DESCRIPTION OF THE PREFERRED EMBODIMENTS

The present invention relates to protein biomarkers for Alzheimer's disease, whereby lack of detection, detection, and/or the quantity of a first protein biomarker in a biological sample from Alzheimer's disease patients correlates with significant differences in the quantities of other protein biomarkers of Alzheimer's disease. When Alzheimer's disease patients and age-matched normal control subjects are each placed into separate categories based on whether they do or do not have detectable quantities of the first protein biomarker, the protein identities of, and the differences in the quantities of the first protein biomarker and/or one or more other protein biomarkers in the biological sample provide opportunities: to improve sensitivity and specificity of differential diagnosis. To measure disease severity and monitor drug response. To monitor drug clinical trial stratification of patients. To indicate differences in neuronal degeneration mechanisms in the patients. To measure the activity of these mechanisms. To determine which of these mechanisms predominates. To determine which biomarkers and mechanisms measure the severity of Alzheimer's disease in the patients. To discover new targeted therapies. To develop companion diagnostics.

More particularly, a preferred embodiment of the present invention relates to the identification of the relationships between two or more of an Apolipoprotein E4 protein, an Apolipoprotein E3 protein, an Apolipoprotein A-IV Protein, a Transthyretin protein, a Complement C3c1 protein, a Complement C3c2a protein, a Complement C3dg protein, a Complement Factor Bb protein, A Complement Factor H/Hs protein, a Complement Factor I protein, an Immunoglobulin protein, a Haptoglobin protein, and/or an Inter-alpha Trypsin Inhibitor Heavy Chain (H4) related 35 KD protein, as biomarkers for distinguishing between different categories or types of Alzheimer's disease, and for early detection, screening, diagnosis, differential diagnosis, and monitoring of disease severity and disease mechanisms of patients with Alzheimer's disease (AD), Alzheimer's disease Like (AD-Like) dementias, Amyotrophic Lateral Sclerosis (ALS, Lou Gehrig's disease), and Parkinson's disease. In this embodiment, the lack of detection, detection, and/or the quantity of the first protein biomarker, an Apolipoprotein E4 protein, and the quantities of one or more of an Apolipoprotein E3 protein, an Apolipoprotein A-IV Protein, a Transthyretin protein, a Complement C3c1 protein, a Complement C3c2a protein, a Complement C3dg protein, a Complement Factor Bb protein, A Complement Factor H/Hs protein, a Complement Factor I protein, an immunoglobulin protein, a Haptoglobin protein, and/or an Inter-alpha Trypsin Inhibitor Heavy Chain (H4) related 35 KD protein, are employed for distinguishing between different categories or types of Alzheimer's disease, and for early detection, screening, diagnosis, differential diagnosis, and monitoring of disease severity and disease mechanisms of patients with Alzheimer's disease (AD), Alzheimer's disease like (AD-Like) dementias, Amyotrophic Lateral Sclerosis (ALS, Lou Gehrig's disease), and Parkinson's disease (PD).

The method for identification of an Apolipoprotein E4 protein as a biomarker for Alzheimer's disease is based on the comparison of 2D gel electrophoretic images of serum obtained from human subjects with and without diagnosed Alzheimer's disease.

2D gel electrophoresis has been used in research laboratories for biomarker discovery since the 1970′s (7-16). In the past, this method has been considered highly specialized, labor intensive and non-reproducible. Only recently with the advent of integrated supplies, robotics, and software, combined with bioinformatics, has progression of this proteomics technique in the direction of diagnostics become feasible. The promise and utility of 2D gel electrophoresis is based on its ability to detect changes in expression of intact proteins and to separate and discriminate between specific intact protein isoforms that arise due to variations in amino acid sequence and/or post-synthetic protein modifications such as phosphorylation, ubiquitination, conjugation with ubiquitin-like proteins, acetylation, glycosylation, and proteolytic processing. These are critical features in cell regulatory processes that are differentially expressed in blood serum biomarkers in neurodegenerative diseases, including Alzheimer's and Parkinson's diseases, and ALS (Goldknopf, I. L. et al. U.S. Utility patent application Ser. No. 11/507,337, and 17-19).

There are few comparable alternatives to 2D gel electrophoresis for tracking changes in intact protein expression patterns related to disease. Furthermore, the introduction of high sensitivity fluorescent staining for ultra high sensitivity visualization of characteristic, recognizable protein spot patterns, digital image processing, and computerized quantitative image analysis has greatly amplified and simplified the detection of unique species and the quantification of proteins. By using known protein standards as landmarks within each gel run, computerized analysis can detect unique differences in protein expression and modifications between two samples from the same individual or between several individuals.

Separated intact protein spots in the 2D gels that of interest can be excised from the gels and the proteins can then be identified by in-gel proteolytic digestion followed by mass spectrometric analysis. This includes matrix assisted laser desorption time of flight mass spectroscopy (MALDI-TOF MS) based peptide mass fingerprinting and database searching, and/or liquid chromatography with tandem mass spectrometry (LC MS/MS) to provide partial sequencing of individual peptides to confirm identification of the proteins

The identification of an Apolipoprotein E4 protein, an Apolipoprotein E3 protein, an Apolipoprotein A-IV Protein, a Transthyretin protein, a Complement C3c1 protein, a Complement C3c2a protein, a Complement C3dg protein, a Complement Factor Bb protein, A Complement Factor H/Hs protein, a Complement Factor 1 protein, a Haptoglobin protein, an immunoglobulin protein, and an Inter-alpha Trypsin Inhibitor Heavy Chain (H4) related 35 KD protein as biomarkers of neurodegenerative disease was based on a quantitative comparison of the 2D gel electrophoretic images of blood serum samples obtained from 75 normal/Controls, 115 Alzheimer's disease patients (AD), 12 Parkinson's disease patients (PD), and 12 patients with AD-Like and Mixed dementias including Frontotemporal dementia (FTD); Lewy body dementia (LBD); Corticalbasal Ganglionic degeneration (CBGD); Alcohol related dementia; Semantic dementia; Vascular (Multi-infarct) dementia; Stroke (CVA); Post-irradiation Encephalopathy and Seizures; Alzheimer's disease combined with Vascular (Multi-Infarct) dementia; Alzheimer's disease combined with Lewy body dementia; Parkinson's disease combined with Lewy body dementia; Alzheimer's and Parkinson's disease combined with Lewy body dementia; Frontotemporal dementia combined with Chronic Inflammatory Demyelinating Polyneuropathy; and Thalamic CVA combined with HX of Lung CA.

### Sample Collection and Preparation

Sample collection and storage have been performed in many different ways depending on the type of sample and the conditions of the collection process. In the present study, serum samples were collected, aliquoted and stored in a −80° C. freezer before analysis.

In a preferred embodiment of the invention, the serum samples were removed from −80° C. and placed on ice for thawing. To each 100 μL of sample, 100 μL of LB-2 buffer (7M urea. 2M Thiourea, 1% DTT, 1% Triton X-100, 1× Protease inhibitors, and 0.5% Ampholyte pH 3-10) was added and the mixture vortexed. The sample was incubated at room temperature for about 5 minutes.

### Two Dimensional Gel Electrophoresis of Samples

Separation of the proteins in the serum samples was then performed using 2D gel electrophoresis. The 2D gel electrophoretic images were obtained, compared and analyzed as described in the U.S. Utility patent application Ser. No. 11/411,659 filed Apr. 26, 2006 and entitled “Assay for Neuromuscular Diseases” by inventors Goldknopf I L, et al., and as described in the U.S. Utility patent application Ser. No. 11/4487,715 filed Jul. 17, 2006 and entitled “Assay for ALS and ALS-like Disorders” by inventors Goldknopf IL, et al., and as described in the U.S. Utility patent application Ser. No. 11/503,881 filed Aug. 14, 2006 and entitled “Assay for Differentiating Alzheimer's and Alzheimer's-like Disorders” by inventors Goldknopf I L, et al., incorporated herein by reference. A protein assay was performed on the sample to determine total protein content in μg.

Based on the total protein content in the sample, an aliquot of approximately 100 μg of the protein was suspended in a total volume of 184 μL of IEF loading buffer containing 1 μL Bromophenol Blue as a marker to trace the progress of the electrophoresis. Each sample was loaded onto an 11 cm IEF strip (Bio-Rad), pH 5-8, and overlaid with 1.5-3.0 ml of mineral oil to minimize the sample buffer evaporation. Using the PROTEAN® IEF Cell, an active rehydration was performed at 50V and 20° C. for 12-18 hours.

IEF strips were then transferred to a new tray and focused for 20 min. at 250V followed by a linear voltage increase to 8000V over 2.5 hours. A final rapid focusing was performed at 8000V until 20,000 volt-hours were achieved. Running the IEF strip at 500V until the strips were removed finished the isoelectric focusing process.

Isoelectric focused strips were incubated on an orbital shaker for 15 mm with equilibration buffer (2.5 ml buffer/strip). The equilibration buffer contained 6M urea, 2% SDS, 0.375M HCl, and 20% glycerol, as well as freshly added DTT to a final concentration of 30 mg/ml. An additional 15 mm incubation of the IEF strips in the equilibration buffer was performed as before, except freshly added iodoacetamide (C₂H₄INO) was added to a final concentration of 40 mg/ml. The IPG strips were then removed from the tray using clean forceps and washed five times in a graduated cylinder containing the Bio Rad running buffer 1× Tris-Glycine-SDS.

The washed IEF strips were then laid on the surface of Bio Rad pre-cast CRITERION SDS-gels 8-16%. The IEF strips were fixed in place on the gels by applying a low melting agarose. A second dimensional separation was applied at 200V for about one hour. After electrophoresis, the gels were carefully removed and placed in a clean tray and washed twice for 20 minutes in 100 ml of pre-staining solution containing 10% methanol and 7% acetic acid.

### Staining and Analysis of the 2D Gels

The gels were stained with SyproRuby™ (Bio-Rad Laboratories) fluorescent protein stain and subjected to fluorescent digital image analysis in an FX Imager (Bio-Rad Laboratories). The protein patterns of the serum samples were analyzed using PDQUEST™ (Bio-Rad Laboratories) image analysis software.

The 2D gel patterns of the 75 serum samples collected from normal control subjects were compared with each other pursuant to the methodology described in the U.S. Utility patent application Ser. No. 11/411,659 filed Apr. 26, 2006 and entitled “Assay for Neuromuscular Diseases” by inventors Goldknopf I L, et al., and as described in the U.S. Utility patent application Ser. No. 11/4487,715 filed Jul. 17, 2006 and entitled “Assay for ALS and ALS-like Disorders” by inventors Goldknopf I L, et al., and as described in the U.S. Utility patent application Ser. No. 11/503,881 filed Aug. 14, 2006 and entitled “Assay for Differentiating Alzheimer's and Alzheimer's-like Disorders” by inventors Goldknopf I L, et al., incorporated herein by reference. The 75 normal individual blood serum samples all gave similar 2D gel protein patterns.

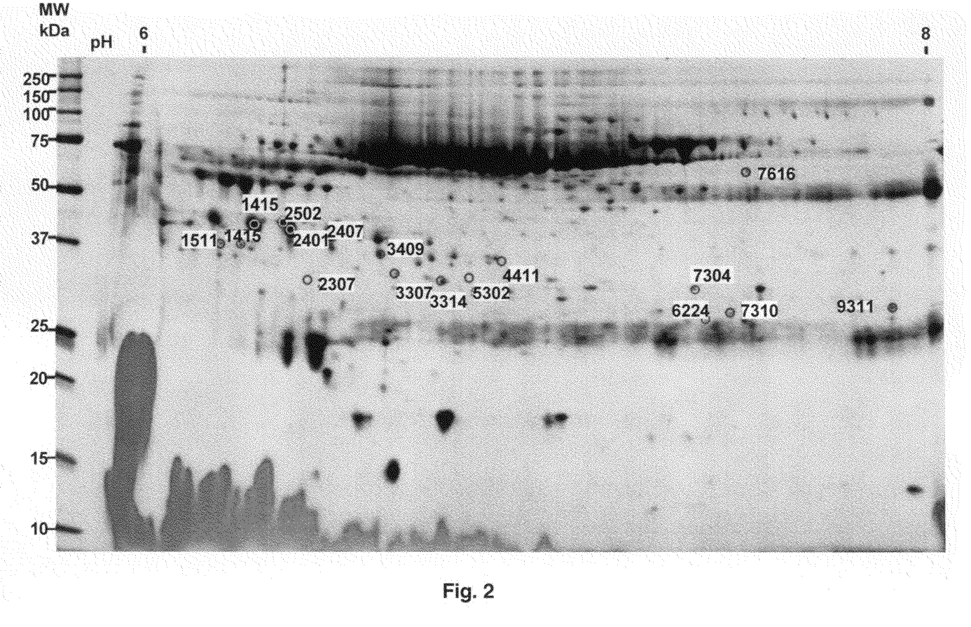

These normal protein expression patterns were then compared to the gel patterns obtained with blood serum samples from the 115 Alzheimer's disease (AD) patients, 12 Parkinson's disease patients (PD), and 12 patients with AD-Like and Mixed dementias including: Frontotemporal dementia (FTD); Lewy body dementia (LBD); Alcohol related dementia; Semantic dementia; Vascular (Multi-infarct) dementia; Stroke (CVA); Post-irradiation Encephalopathy and Seizures; Alzheimer's disease combined with Vascular (Multi-Infarct) dementia; Alzheimer's disease combined with Lewy body dementia; Parkinson's disease combined with Lewy body dementia; Alzheimer's and Parkinson's disease combined with Lewy body dementia; Frontotemporal dementia combined with Chronic Inflammatory Demyelinating Polyneuropathy; and Thalamic CVA combined with HX of Lung CA. When the gel patterns of AD patients were compared to the gel patterns of normal subjects, protein spots N5302, N3314, N3307, N7310, N9311, N1511, N7616, N4411, N 1416, N1514, N2401, N2407, N3409, N6224, N2502, and N2307, of particular interest, were identified as shown in FIG. 2, and selected for further investigation. Protein spots N5302, N3314, N3307, N7310, N9311, N1511, N7616, N4411, N1416, N1514, N2401, N2407, N3409, N6224, N2502, and N2307 were quantitated by stain intensity in each of the normal and disease patient groups of serum samples.

In order to assess the reproducibility of the 2D gels and staining, 75 nanograms of bovine serum albumin (BSA) was run on 9 separate 2D gels. The gels were stained with SYPRO RUBY and the 5 spots resolved in the BSA region of the gel were then subjected to quantitative analysis using PDQUEST™ and the Gaussian Peak Value method. The results shown in Table 1 illustrate that the electrophoretic patterns were reproducible and the reproducibility (% Coefficient of Variation=% CV) was independent of the spot amount over the range tested (2.9-38.6 ng/spot).

TABLE 1
Reproducible Quantitation of Bovine Serum Albumin
(BSA) Standard (n = 9)

| | Spot # | | | | |
|---|---|---|---|---|---|
| Replicate # | 9901 | 9902 | 9904 | 9905 | 9906 |
| 1 | 332 | 1152 | 2612 | 739 | 229 |
| 2 | 246 | 974 | 2694 | 513 | 167 |
| 3 | 336 | 1065 | 2354 | 668 | 225 |
| 4 | 311 | 1272 | 3482 | 713 | 198 |
| 5 | 351 | 1168 | 2724 | 733 | 245 |
| 6 | 268 | 1059 | 2753 | 622 | 184 |
| 7 | 452 | 1630 | 4000 | 946 | 281 |
| 8 | 405 | 1195 | 2752 | 870 | 274 |
| 9 | 258 | 1050 | 2716 | 699 | 189 |
| AVG | 329 | 1174 | 2899 | 723 | 221 |
| STDEV | 68 | 193 | 510 | 127 | 40 |
| % CV | 21% | 16% | 18% | 18% | 18% |
| ng/spot | 4.4 | 15.6 | 38.6 | 9.6 | 2.9 |

### Reproducibility of Quantitation in 9 Gels—PDQuest Gaussian Peak Value of the Major Components of BSA

As shown in FIG. 1A, 2D gel electrophoresis of human blood serum, fluorescent staining with SyproRuby, and digital imaging provides a broad dynamic concentration range of protein spots, which are illustrated by the indicated spots with concentrations ranging from a low of 55 ppm spot density to a high of 15,789 ppm spot density (white arrows). Triplicate analysis with the same blood serum sample shows good reproducibility, with a coefficient of variation=13.8% for the triplicate analysis of the indicated spot (FIG. 1B). Table 2 illustrates the reproducibility of quantitation of 13 different spots from 2D gel electrophoresis of human blood serum, with decreasing concentrations over the full dynamic range of the assay, illustrated with protein spots ranging in spot density from a low of 72 ppm to a high of 13,542 ppm, with a coefficients of variation ≦20% for replicates of 14 gels run on different days with different technicians, independent of the concentrations of the protein spots within that range. The limit of detection (LOD) is at a 10 fold lower concentration than the bottom of that range, or 100 pg/spot˜5-10 PPM

TABLE 2
Reproducible Quantitation of 13 Different Protein Spots
(n = 14, Range 72 ppm-13,542 ppm)

| | | | | | Coefficient | |
|---|---|---|---|---|---|---|
| | | | | Std | of | |
| Biomarker | N | Mean | +/− | Error | Variation | ≦20% |
| M1 | 14 | 13542 | | 711 | 20 | \| |
| M2 | 14 | 3853 | | 140 | 14 | \| |
| M3 | 14 | 14 [symbol] 3 | | 52 | 14 | \| |
| M4 | 14 | 10 [symbol] 5 | | 49 | 18 | \| |
| M5 | 14 | 678 | | 28 | 15 | \| |
| M6 | 14 | 655 | | 33 | 19 | \| |
| M7 | 14 | 595 | | 31 | 19 | \| |
| M8 | 14 | 469 | | 26 | 20 | \| |
| M9 | 14 | 359 | | 16 | 17 | \| |
| M10 | 14 | 209 | | 11 | 20 | \| |
| M11 | 14 | 129 | | 5 | 15 | \| |
| M12 | 14 | 106 | | 6 | 20 | \| |
| M13 | 14 | 72 | | 4 | 19 | ⇓ |

LOD = 100 pg/spot = ~5-10 ppm [symbol]
indicates data missing or illegible when filed

### The Isolation and Identification of the Protein Spots

Protein spots N5302 N3314, N3307, N7616, N4411, N1416, N7310, N9311, N1511, N1514, N2401, N2407, N3409, N6224, N2502, and N2307, were carefully excised, in-gel digested with trypsin, and subjected to mass fingerprinting/sequence analysis by high performance liquid chromatography/tandem mass spectrometry (LC-MS/MS) and expert database searching.

Tandem mass spectrometry provides a powerful means of determining the structure and identity of proteins and peptides. The unknown tryptic peptide is first separated and purified by liquid chromatography and then the effluent from the separation is vaporized by electrospray, separated in a mass spectrometer and then bombarded with high-energy electrons causing it to fragment in a characteristic manner, indicative of its amino acid sequence. The fragments, which are of varying mass and charge, are then passed through a magnetic field and separated according to their mass/charge ratios. The resulting characteristic fragmentation pattern of the unknown peptide is used to identify its amino acid sequence.

A protein can often be unambiguously identified by an LC MS/MS analysis of its constituent peptides (produced by either chemical or enzymatic treatment of the sample).

Following differential expression analysis, protein spots N5302 N3314, N3307, N7616, N4411, N1416, N7310, N9311, N1511, N1514, N2401, N2407, N3409, N6224, N2502, and N2307, were carefully excised from the gel for identification. Excised gel spots of proteins N5302 N3314, N3307, N7616, N4411, N1416, N7310. N9311, N1511, N1514, N2401, N2407, N3409, N6224 N2502, and N2307, were de-stained by washing the gel spots twice in 100 mM NH₄HCO₃ buffer, followed by soaking the gel spots in 100% acetonitrile for 10 minutes. The acetonitrile was aspirated before adding the trypsin solution. Typically, a small volume of trypsin solution (approximately 5-15 μg/ml trypsin is added to the de-stained gel spots and incubated at 3 hours at 37° C. or overnight at 30° C. The digested peptides were extracted, washed, desalted and subjected to liquid chromatography followed by tandem mass spectral analysis to identify the protein spots.

Tandem mass spectrometry of tryptic peptides provides a powerful means of determining the structure and identity of proteins. The unknown tryptic peptides from the digestion are extracted from the gel and first separated and purified by liquid chromatography and then the effluent from the separation is vaporized by electrospray, separated in a mass spectrometer and then bombarded with high-energy electrons causing the peptides to fragment it in a characteristic manner, indicative of their amino acid sequences. The fragments, which are of varying mass and charge, are then passed through a magnetic field and separated according to their mass/charge ratios. The resulting characteristic fragmentation patterns of the unknown peptides are used to identify the amino acid sequence of the protein spot from which they were obtained. Those of skill in the art are familiar with mass spectral analysis of digested peptides. The mass spectral analysis was conducted on a Micromass LC QTOF (Waters). Peptide fragmentation patterns were obtained from the tryptic in-gel digests of the protein spots and the patterns were subjected to public database searches using the GenBank and dbEST databases maintained by the National Center for Biotechnology Information (hereinafter referred to as the NCBI database). Those of skill in the art are familiar with searching databases, like the NCBI database. The NCBI database search results were displayed with the best matched amino acid sequences of the identified peptides and the protein accession of number the protein sequence they were derived from. Biomarkers identified by LC-MS/MS of the in-gel tryptic peptide digests are listed.

The NCBI database search results were displayed with the best matched amino acid sequences of the identified tryptic peptides and the protein accession numbers of the proteins sequences they were derived from. For protein spots N5302 N3314, N3307, N7616, N4411, N1416, N7310, N9311, N1511, N1514, N2401, N2407, N3409, N6224, N2502, and N2307, the proteins identified by the NCBI database search were: N5302, is an Apolipoprotein E4 protein (Precursor SEQ ID NO. 1, N5302 SEQ ID NO. 2); N3314, an Apolipoprotein E3 (Precursor SEQ ID NO. 3, N3314 SEQ ID NO. 4); N3307, a Transthyretin “Dimer” protein (N3307 SEQ ID NO. 5); 3 Complement C3 proteins; N7310, a Complement C3c1 protein (Precursor SEQ ID NO. 6, N7310 SEQ ID NO. 7); N9311, a Complement C3c2a protein (Precursor SEQ ID NO. 6, N9311 SEQ ID NO. 8,); and N1511, a Complement C3dg protein (Precursor SEQ ID NO. 6, N1511 SEQ ID NO. 10, alternate precursor SEQ ID NO. 9, N1511 alternate SEQ ID NO. 11); N7616, a Complement Factor Bb protein (N7616 SEQ ID NO. 12); N4411, a Complement Factor H/Hs protein (Precursor SEQ ID NO. 13, alternate precursor SEQ ID NO. 14, N4411 SEQ ID NO. 15); and N2307, An Inter-alpha Trypsin Inhibitor protein (Heavy Chain H4 Related Precursor Protein SEQ ID NO. 16, N2307 Heavy Chain H4 isoform 1 SEQ ID NO. 17, N2307 Heavy Chain H4 alternate isoform 2 SEQ ID NO. 18); Four Haptoglobin proteins; N1514 N2401, N2407, and N3409, electrophoretic variants of a Haptoglobin HP-1 protein (N1514 N2401, N2407, N3409 SEQ ID NO. 19); N1416, Complement Factor I protein (N1416 SEQ ID NO. 20); N6224, an Immunoglobulin Light Chain protein (N6224 SEQ ID NO. 21); and N2502, an Apolipoprotein A-IV protein (N2502 SEQ ID NO. 22).

### Biostatistical Analysis

Statistical significance of differences in individual biomarker blood serum concentrations between different patient and control groups is performed using methods well known in the art, Dot Box and Whiskers plots, analysis of variance, and Receiver Operator Characteristics, employing a standard off the shelf software package, “Analyze-it” in Microsoft XL. Box and Whisker plots give a visual representation of non-parametric descriptive statistics. The central “box” (FIG. 37) represents the distance between the first and third quartiles (inter quartile range or IQR), with the median marked as the horizontal line inside the box. The notch in the box represent the 95^{th} % confidence interval around the median (the 50th percentile); thus groups that display non-overlapping notches can be considered statistically different (p<0.05). The minimum value is the origin of the leading “whisker” and the maximum value is the limit of the trailing “whisker”. All values are plotted individually (Dots) and those values outside the whiskers are considered possible outliers, presented either as circle (far outlier) or plus sign (near outliers).

### Receiver Operating Characteristic (ROC) Curve

The diagnostic performance of a test or the accuracy of a test to discriminate diseased cases from normal cases is evaluated using Receiver Operating Characteristic (ROC) curve analysis. ROC curves can also be used to compare the diagnostic performance of two or more laboratory or diagnostic tests. In ROC curve the true positive rate (Sensitivity) is plotted in function of the false positive rate (1—Specificity) for different cut-off points. Each point on the ROC plot represents a sensitivity/specificity pair corresponding to a particular decision threshold. A test with perfect discrimination (no overlap in the two distributions) has a ROC plot that passes through the upper left corner (100% sensitivity, 100% specificity). Therefore the closer the ROC plot is to the upper left corner, the higher the overall accuracy of the test (64).

Differential Expression of Protein spots N5302, N3314, N3307, N7616, N4411, N1416, N7310, N9311, N1511, N1514, N2401, N2407, N3409, N6224, N2502, and N2307 in Age Matched Normal Control Subjects, and Patients Diagnosed with Alzheimer's Disease, with Parkinson's disease, and with AD-Like, and/or Mixed Disorders

The blood serum concentrations of Apolipoprotein E4 protein spot N5302 (FIG. 3, Table 3), Apolipoprotein E3 protein spot N3314 (FIG. 5, Table 5), Transthyretin “Dimer” protein N3307 (FIG. 8, Table 8), Complement Factor H/Hs protein spot N4411 (FIG. 10, Table 10), Complement Factor Bb protein spot N7616 (FIG. 12, Table 12), Complement C3c1 protein spot N7310 (FIG. 14, Table 14), Complement C3c2a protein spot N9311 (FIG. 14, Table 14), Complement C3dg protein spot N1511 (FIG. 14, Table 14), C3Sum=N7310+N9311+N1511 (FIG. 14, Table 14), Haptoglobin HP-1 proteins N1514, N2401, N2407, and N3409 (FIGS. 23, 24), Total Haptoglobin HP-1=N1514+N2401+N2407+N3409 (FIG. 25, Table 19), Inter-alpha Trypsin Inhibitor Heavy Chain H4 related 35 KD protein N2307 (FIG. 26, Table 20), Immunoglobulin Light Chain Protein N6224 (FIG. 29, Table 22), and Apolipoprotein A-IV protein N2502 (FIG. 31, Table 24) were all determined by triplicate 2D gel analysis of individual blood serum samples from 75 age matched normal controls, 115 Alzheimer's disease (AD) patients, 12 Parkinson's disease patients (PD), and 12 patients with AD-Like or Mixed dementias, 12 patients with AD-Like and Mixed disorders, including: Frontotemporal dementia (FTD), Frontotemporal dementia combined with Chronic Inflammatory Demyelinating polyneuropathy, Lewy body dementia (LBD), Vascular (Multi-infarct) dementia, Thalamic CVA combined with HX or Lung CA, Post-irradiation Encephalopathy, Seizures, Alcohol related dementia, Semantic dementia, Memory Dysfunction, neuro exam “Normal,” and Corticalbasal Ganglionic Degeneration (CBGD).

### Apolipoprotein E4 and Apolipoprotein E3

Shown in FIG. 3 and Table 3 are the differences in the blood serum concentration of Apolipoprotein E4 protein N5302 in AD, PD and ADL (Alzheimer-like disorders) as percent difference from age matched normal controls (AMC). The application of Apolipoprotein E4 (spot N5302) as a single biomarker to differentiate between Alzheimer's disease patients and age-matched control (AMC) subjects shows more trend towards specificity (74.7%).

The Apolipoprotein E4 protein N5302 is the protein product of the Apo E ε4 gene allele. The Apo E ε4 gene allele is known to be associated with increased risk of dementia, and is inherited as one of three Apo E gene alleles, termed ε2, ε3, and ε4, with mean frequencies in the general population of about 8%, 78%, and 14%, respectively (3). The degree of risk of dementia conferred by Apo E ε4 allele rises in a “gene dose” dependent manner (4), increasing with the number of Apo E ε4 alleles inherited, from: ε4 non-carriers; to ε4/ε3 and ε4/ε2 hetero-zygotes; to ε4/ε4 homo-zygotes (5), all capable of developing Alzheimer's disease, although those lacking Apo E ε4 allele have the least risk of developing AD, and also may tend to get the disease at a later age of onset (6). In a preferred embodiment of the invention, those Alzheimer's disease patients and age matched normal controls who have detectable levels of Apolipoprotein E4 protein in their blood serum (N5302>0) are assumed to be either Apo E ε4/ε3 or ε4/ε2 hetero-zygotes, or ε4/ε4homo-zygotes, and to not be Apo E ε4 non-carriers. Also in a preferred embodiment of the invention those Alzheimer's disease patients and age matched normal controls who have no detectable levels of Apolipoprotein E4 protein in their blood serum (N5302=0) are assumed to be Apo E ε4 non-carriers, although there may be some individuals in this group who have the Apo E ε4 allele in their genome but it is unexpressed as protein or expressed below the level of detection of the 2D gel electrophoresis method employed.

In the preferred embodiment of the invention, the detection, or a lack of detection of Apolipoprotein E4 protein N5302 expression, as measured in blood serum, whether Apolipoprotein E4 protein concentration is detected (N5302>0), or is not detected (N5302=0), is determined and its effect upon the expression of other blood serum biomarkers of Alzheimer's disease, measured as changes in blood serum concentration, are used to measure differences in the form that Alzheimer's disease takes in the patient.

As shown in FIGS. 3A and 4A, and accompanying Tables 3 and 4, AD patients have significantly higher blood serum concentrations of Apolipoprotein E4 protein spot N5302 than age matched normal controls (ANOVA-P<0.0001), Parkinson's disease patients, and patients with Alzheimer's disease-like dementias. In the case of Alzheimer's disease (AD) vs. age matched normal controls (AMC), using a cutoff of N5302>0; the separation between the AD and AMC groups is less sensitive for detection of Alzheimer's disease (FIG. 3b, Table 3, Receiver Operator Characteristics, ROC Sensitivity=55.1%, ROC-P<0.0001) and more specific for detection of age matched normal controls (FIG. 3b, Table 3, ROC Specificity=74.7%, ROC-P<0.0001), reflecting the increased risk of Alzheimer's disease in those who have the Apo E ε4 allele and who express the allele as protein in blood serum (Apolipoprotein E4 protein spot N5302>0). Moreover, in AD and AMC individuals who have detectable levels of Apolipoprotein E4 protein spot N5302 in their blood serum (N5302>0), the level of Apolipoprotein E4 protein spot N5302 is significantly higher in Alzheimer disease patients than in the age matched controls (FIG. 4a, N5302>0, ANOVA-P<0.0001). When Apolipoprotein E4 protein N5302 is detected in blood serum (N5302>0), the separation between the AD and AMC groups is sensitive for detection of Alzheimer's disease (FIG. 4B, Table 4, ROC Sensitivity=64.2%, ROC-P<0.0030) but not specific for age matched normal controls ((FIG. 4B, Table 4, ROC Specificity=50.7%, ROC-P<0.0030). This indicates that in addition to its detection or lack of detection, the level of expression of Apolipoprotein E4 protein spot N5302 is also a significant factor, in that an increased level of Apolipoprotein E4 protein spot N5302 demonstrates significant sensitivity for detection of Alzheimer's disease. Also, the reduced specificity reflects increased risk and/or undiagnosed Alzheimer's disease in the age matched normal controls who express the Apo E ε4 allele product Apolipoprotein E4 protein N5302 in blood serum (N5302>0).

As shown in FIG. 5A and Table 5, age matched normal control subjects have the highest blood serum concentrations of the Apo E ε3 allele protein product, Apolipoprotein E3 protein spot N3314. Alzheimer's disease patients, patients with AD-Like and Parkinson's disease patients have significantly lower concentrations of Apolipoprotein ε3 protein spot N3314 (ANOVA-P<0.0001), than age-matched normal control (AMC). The reduced level of Apolipoprotein E3 protein spot N3314 in AD is equally sensitive for detection of Alzheimer's disease and specific for age matched normal controls (FIG. 5B, Table 5, ROC Sensitivity=64.1%, ROC Specificity=64.0, ROC-P<0.0001).

However, as shown in FIG. 6A and Table 6, when Alzheimer's disease patients and age matched normal controls are compared on the basis of whether or not Apolipoprotein E4 protein is detected in blood serum (N5302>0 vs. N5302=0, respectively), the Alzheimer's disease patients with detectable blood serum levels of Apolipoprotein E4 protein (N5302>0) have significantly lower expression of Apolipoprotein E3 protein N3314 in blood serum than the Alzheimer's disease patients with no detectable blood serum levels of Apolipoprotein E4 protein (FIG. 6a N5302=0).

When the potential utility for diagnosis of Alzheimer's disease is measured by plotting Receiver Operator Characteristics of blood serum concentrations of Apolipoprotein E3 protein N3314 as a function of whether Apolipoprotein E4 protein N5302 is detected (N5302>0) or not detected (N5302=0) in blood serum (FIG. 7A, Table 7a, b), it is readily apparent that when Apolipoprotein E4 protein spot N5302 was detected (N5302>0) in the blood serum, the distinguishing of Alzheimer's disease patients from age matched normal controls on the basis of reduced blood serum concentration of Apolipoprotein E3 protein spot N3314 was accomplished with significant sensitivity and specificity (Table 7b, Sensitivity=68.2%, Specificity=68.1%, ROC-P<0.0001; AUC=0.76±0.033). Conversely, when Apolipoprotein E4 protein spot N5302 was not detected (N5302=0) in the blood serum, significantly less sensitivity and specificity was obtained by measuring the concentration of Apolipoprotein E3 spot N3314 (Table 7a, Sensitivity=54.2% Specificity=53.8% ROC-P<0.0004; AUC=0.60±0.033).

Thus, in active Alzheimer's disease, decreased expression of Apo E ε3 wild type allele gene product, the Apolipoprotein E3 protein spot N3314, in blood serum has clinical diagnostic utility, when the detection or lack of detection in blood serum of the Alzheimer's disease risk gene allele Apo E ε4 protein product, Apolipoprotein E4 protein spot N5302 is also taken into account.

Results similar to that obtained for Apolipoprotein E3 protein spot N3314 were also obtained for Transthyretin “Dimer” protein spot N3307 (see FIG. 8, Table 8; and FIG. 9, Table 9).

In a preferred embodiment of the invention, the lack of detection or the detection, and the quantity of Apolipoprotein E4 protein spot N5302, is employed combined with the concentrations of Apolipoprotein E3 protein spot N3314 and Transthyretin “Dimer” protein N3307 in blood serum wherein: Concentrations of Apolipoprotein E3 protein spot N3314 and Transthyretin “Dimer” protein spot N3307 in blood serum that are significantly below the ranges of age matched normal controls, with detection of Apolipoprotein E4 protein spot N5302, and with concentrations of Apolipoprotein E4 protein spot N5302 significantly above the range of age matched normal controls, are indicated for sensitive and specific detection of Alzheimer's disease.

For the purposes of the preferred embodiment of this invention, the known association of Apolipoprotein E protein and Transthyretin protein into neurofibrillary tangles and senile plaques, as well as the neuroprotective role of Apolipoprotein E3 against oxidative stress and related signals for apoptosis, indicate significant differences in the mechanisms of neuronal degeneration between these two forms of Alzheimer's disease (wherein either N5302=0 or N5302>0, FIGS. 34-36).

### Complement Factor Bb Protein and Complement Factor H/Hs Protein

Also in a preferred embodiment of the invention, a lack of detection N5302=0), or the detection (N5302>0) and the quantity of Apolipoprotein E4 protein N5302 as measured in blood serum, is determined and its effect upon the expression of Complement Factor H/Hs protein N4411 and Complement Factor Bb protein N7616 is also determined.

Complement Factor H/Hs protein N4411 is significantly up-regulated in the blood serum of patients with Alzheimer's disease and Parkinson's disease, but not in patients with AD-like and Mixed dementias, as compared to age matched normal controls (AMC) (FIG. 10A, Table 10, ANOVA-P<0.0040). Complement Factor Bb protein N7616 is also up-regulated in the blood serum of patients with Alzheimer's disease and Parkinson's disease, and in patients with AD-like and Mixed dementias as well, when compared to age matched normal controls, but the up-regulation in Alzheimer's disease lacks statistical significance (FIG. 12A, Table 12, ANOVA-P>0.110).

In the case of Alzheimer's disease (AD) vs. age matched normal controls (AMC), using a cutoff for N4411 of AD>261 ppm, the separation between AD and AMC groups is equally sensitive and specific (FIG. 10B, Table 10, Sensitivity=57.4%, Specificity=57.3%, ROC-P<0.0002), whereas using a cutoff value of AD>233ppm, Complement Factor Bb protein N7616 demonstrated significantly less sensitivity and specificity (FIG. 12B, Table 12, Sensitivity=52.5%, Sensitivity=52.4%, ROC-P>0.09).

However, when Alzheimer's disease patients and age matched normal controls are compared on the basis of whether or not Apolipoprotein E4 protein spot N5302 is detected in blood serum (N5302>0 vs. N5302=0, respectively), an opposite effect to that on Apolipoprotein E3 protein spot N3314, and Transthyretin “Dimer” protein N3307 was seen. The Alzheimer's disease patients without detectable blood serum levels of Apolipoprotein E4 (N5302=0) had significantly higher expression of both Complement Factor H/Hs protein spot N4411 (FIG. 11A, Table 11, ANOVA-P<0.0001) and Complement Factor Bb protein spot N7616 (FIG. 13A, Table 13, ANOVA-P<0.0006) in blood serum than age matched normal controls. Alzheimer's disease patients with detectable blood serum levels of Apolipoprotein E4 protein (N5302>0) did not have significantly different levels of expression of either Complement Factor H/Hs protein spot N4411 (FIG. 11A, Table 11, ANOVA-P>0.80) nor of Complement Factor Bb protein spot N7616 (FIG. 12A, Table 12, ANOVA-P>0.17) in blood serum than age matched normal controls.

When the potential utility for diagnosis of Alzheimer's disease is measured by plotting Receiver Operator Characteristics of blood serum concentrations of Complement Factor H/Hs protein spot N4411 (FIG. 11B, Table 11) and Complement Factor Bb protein spot N7616 (FIG. 13B, Table 13) as a function of whether Apolipoprotein E4 protein spot N5302 is detected in blood serum, it was found that when Apolipoprotein E4 protein spot N5302 was not detected (N5302=0) in the blood serum, the distinguishing of Alzheimer's disease patients from age matched normal controls on the basis of elevated blood serum concentrations of the Complement Factor H/Hs protein spot N4411 was accomplished with significantly higher sensitivity and specificity (FIG. 11B, Table 11, Sensitivity=62.5%, Specificity=62.1%, cutoff value AD>270 ppm, ROC-P<0.0001). Conversely, when Apolipoprotein E4 protein spot N5302 was detected (N5302>0) in the blood serum, essentially no sensitivity and no specificity was obtained by measuring the concentration of Complement Factor H/Hs protein spot N4411 (FIG. 11B, Table 11, Sensitivity=49.3% Specificity=49.3%, cutoff value AD>273 ppm, ROC-P<0.22). This is also an opposite effect to what was observed for Apolipoprotein E3 protein N3314 and Transthyretin “Dimer” protein spot N3307. Furthermore, similar results were obtained with Complement Factor Bb protein spot N7616 (FIG. 13B, Table 13, N5302=0, Sensitivity 55.6%, Specificity=55.8%, cutoff value AD>237 ppm ROC-P<0.0040; vs. N5302>0, no Sensitivity 50.7%, no Specificity=49.3%, cutoff value AD>229 ppm ROC-P>0.06).

Thus, in active Alzheimer's disease, increased expression of Complement Factor H/Hs protein N4411 and Complement Factor Bb protein N7616 in blood serum has clinical diagnostic utility, when the detection or lack of detection in blood serum of the Alzheimer's disease risk gene allele Apo E ε4 protein product, Apolipoprotein E4 protein spot N5302 is also taken into account. Furthermore, the significantly up-regulated levels of Complement Factor H/Hs protein spot N4411 and Complement Factor Bb protein spot N7616 in Alzheimer's disease patients above age matched normal controls are found only in patients with no detectable Apolipoprotein E4 protein spot N5302 expression (N5302=0), is opposite to the effect of Apolipoprotein E4 protein spot N5302 expression on Apolipoprotein E3 protein spot N3314 and Transthyretin “Dimer” Protein spot N3307. This indicates that reduced levels of Apolipoprotein E3 protein spot N3314 and Transthyretin “Dimer” Protein spot N3307 reflect characteristics of one form of Alzheimer's disease (N5302>0), whereas increased levels of Complement Factor H/Hs protein spot N4411 and Complement Factor Bb protein spot N7616 are characteristics of the other form (N5302=0) of Alzheimer's disease, and this provides for complimentary diagnostic utilities.

In a preferred embodiment of the invention, combining the lack of detection or the detection, and the quantity of Apolipoprotein E4 protein spot N5302, with the concentrations of Apolipoprotein E3 protein spot N3314, Transthyretin “Dimer” protein spot N3307, Complement Factor H/Hs protein spot N4411, and Complement Factor Bb protein spot N7616 in blood serum wherein: Concentrations of Apolipoprotein E3 protein spot N3314 and Transthyretin “Dimer” protein spot N3307 in blood serum that are significantly below the ranges of age matched normal controls when Apolipoprotein E4 protein spot N5302 is detected (N5302>0) and the concentrations of Apolipoprotein E4 protein spot N5302, are indicated for sensitive and specific detection of one form of Alzheimer's disease; and concentrations of Complement Factor H/Hs protein spot N4411 and Complement Factor Bb protein spot N7616 that are significantly above the range of age matched normal controls when Apolipoprotein E4 protein spot N5302 is not detected (N5302=0) are indicated for sensitive and specific detection of another form of Alzheimer's disease (N5302=0); and by detecting both of the types of Alzheimer's disease (wherein N5302=0 and N5302>0) with complementary characteristics, greater sensitivity and specificity is obtained for detection of Alzheimer's disease.

For the purposes of the preferred embodiment of this invention, the known activity of Complement Factor H/Hs protein spot N4411 in releasing Complement Factor Bb protein spot N7616 from the alternate Complement C3 Convertase, indicate significant differences in the mechanisms of neuronal degeneration between the two forms of Alzheimer's disease (N5302=0, N5302>0, FIG. 36).

Complement C3c1, C3c2a, and C3dg

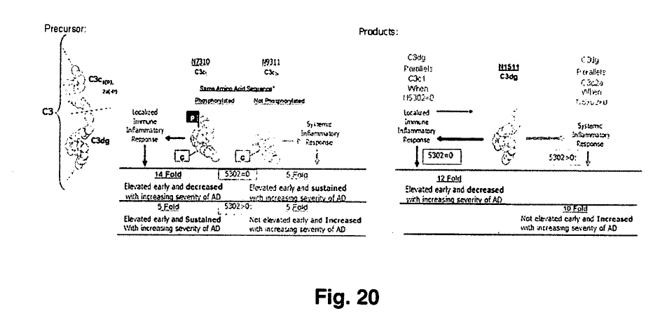

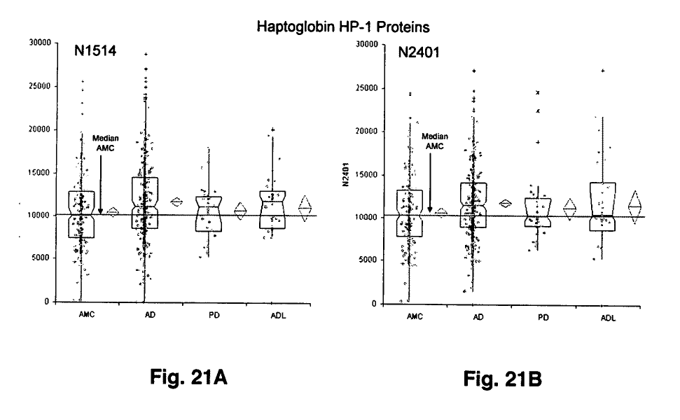

In the preferred embodiment of the invention, lack of detection, detection, detection and/or quantity of Apolipoprotein E4 protein spot N5302, as measured in blood serum (whether Apolipoprotein E4 protein spot N5302 concentration is >0 or=0), is determined and its effect upon the expression of Complement C3c1 phosphoprotein spot N7310, Complement C3c2a protein spot N9311, Complement C3dg protein spot N1511, and C3Sum (N7310+N9311+N1511), are also measured in blood serum. The two isoforms of Complement C3c protein (C3c1 and C3c2a) have the same amino acid sequence (ref. 17) derived from the same locus of Complement C3 parent precursor (SEQ ID NO. 6, FIG. 20, amino acids 749-951). Phosphorylated Complement C3c1 protein spot N7310 (SEQ ID NO. 7) is derived from Complement C3 parent precursor tyrosine phosphorylated during its translation in response to auto-immune antibody stimulation of the neuronal Fc receptor, and non-phosphorylated Complement C3c2a protein spot N9311 (SEQ ID NO. 8) is derived from non-phosphorylated Complement C3 parent precursor in the Classical Complement Pathway of innate inflammation (refs. 17-37, FIGS. 20, 34-37).

Complement C3dg protein spot N1511 is also derived from the Complement C3 parent precursor, but downstream of the locus for Complement C3c1 and C3c2a (SEQ ID NO. 6, amino acids 955-1303). It arises when Complement iC3b is cleaved to make Complement C3c and Complement C3dg (SEQ ID NO. 10). Alternately, Complement C3dg protein spot N1511 (SEQ ID NO. 11) arises from alternate parent protein Similar to C3 (SEQ ID NO. 9, amino acids 902-1256).

As shown in FIG. 14, Table 14, and FIG. 20, Complement C3c1 phosphoprotein N7310 (FIG. 14A, Table 14a), Complement C3c2a protein N9311 (FIG. 14C, Table 14c), Complement C3dg protein N1511 (FIG. 14B, Table 14b), and the C3Sum (N7310+N9311+N1511) (FIG. 14D, Table 14d), were significantly up-regulated (ANOVA-P<0.0001, Table 14) in the blood serum of Alzheimer's and Parkinson's disease patients, compared to age matched normal controls. Complement C3c1 phosphoprotein spot N7310 is not up-regulated in blood serum of patients with AD-like and mixed dementias (FIG. 14A, Table 14a), whereas Complement C3c2a protein spot N9311 is up-regulated in the blood serum of these patients (FIG. 14C, Table 14c), and Complement C3dg protein spot N1511 is up-regulated to a lesser extent than Complement C3c2a protein spot N9311 in the blood serum of these patients (FIG. 14B, Table 14b).

By ROC analysis, Complement C3c1 phosphoprotein spot N7310 (FIG. 15A, Table 15a) Complement C3c2a protein spot N9311 (FIG. 15C, Table 15c), Complement C3dg protein spot N1511 (FIG. 15B, Table 15b), and the C3Sum (N7310+N9311+N1511) (FIG. 15D, Table 15d), showed sensitivities and specificities of discrimination between 115 Alzheimer's disease patients and 75 age matched normal controls as follows:
1. N7310: 55.9% sensitivity, 55.6% specificity (Table 15a, AD>273 ppm, ROC-P<0.0001);
2. N1511: 60.9% sensitivity, 60.9% specificity (Table 15b, AD>105 ppm, ROC-P<0.0001);
3. N9311: 53.9% sensitivity, 53.8% specificity (Table 15c, AD>272 ppm, ROC-P<0.0007);
4. C3Sum: 55.1% sensitivity, 55.1% specificity (Table 15d, AD>710 ppm, ROC-P<0.0001).

Furthermore, in Alzheimer's disease patients, significantly up-regulated levels of Complement C3c1 phosphoprotein spot N7310, C3c2a protein spot N9311 and Complement C3dg protein spot N1511, and C3Sum, above age matched normal controls are found regardless of the detection (N5302>0), or lack of detection (N5302=0), of Apolipoprotein E4 protein spot N5302 expression (FIG. 16; Table 16, ANOVA-P<0.0001). Also, the up-regulation was more pronounced with the Alzheimer's disease patients and age matched controls when Apolipoprotein E4 protein spot N5302 was not detected (N5302=0) in their blood serum than with the Alzheimer's disease patients and age matched controls when Apolipoprotein E4 protein spot N5302 was detected (N5302>0) in their blood serum.
1. N7310: N5302=0, AD=323% of AMC; N5302>0, AD=269% of AMC (Table 16a)
2. N1511: N5302=0, AD=511% of AMC; N5302>0, AD=338% of AMC (Table 16b)
3. C3Sum: N5302=0, AD=295% of AMC; N5302>0, AD=256% of AMC (Table 16b)

The one exception was Complement C3c2a protein spot N9311, where the up-regulation was essentially to the same extent, regardless of whether Apolipoprotein E4 protein spot N5302 was detected in their blood serum.
N9311: N5302=0, AD=196% of AMC; N5302>0, AD=206% of AMC (Table 16a)

Using ROC analysis (FIG. 17, Table 17), these protein biomarkers demonstrate discrimination of AD from age matched normal controls with:
1. N7310: N5302=0; 59.0% sensitivity, 59.0% specificity (Table 17a, AD>309 ppm, ROC-P<0.0006);
2. N7310: N5302>0; 59.2% sensitivity, 59.4% specificity (Table 17a, AD>273 ppm, ROC-P<0.0002);
3. N1511: N5302=0, 63.2% sensitivity, 62.8% specificity (Table 17b, AD>107 ppm, ROC-P<0.0001);
4. N1511: N5302>0, 58.2% sensitivity, 58.0% specificity (Table 17b, AD>106 ppm, ROC-P<0.0001).
5. C3Sum: N5302=0, 56.3% sensitivity, 56.4% specificity (Table 17b, AD>743 ppm, ROC-P<0.0001);
6. C3Sum: N5302>0, 58.2% sensitivity, 58.0% specificity (Table 17b, AD>635 ppm, ROC-P<0.0001).

Again, the only exception in Complement C3c2a protein spot N9311, which only showed sensitivity and specificity, when N5302>0:
1. N9311: N5302=0, 52.1% no sensitivity, 51.9% no specificity (Table 17b, AD>107 ppm, ROC-P<0.03);
2. N9311: N5302>0, 58.2% sensitivity, 58.0% specificity (Table 17b, AD>106 ppm, ROC-P<0.0006).

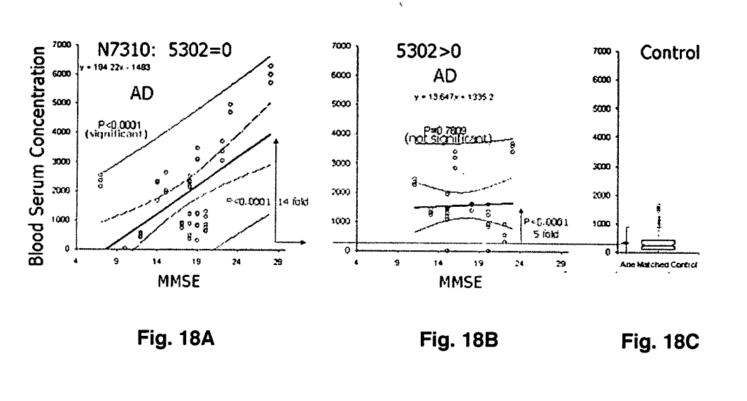

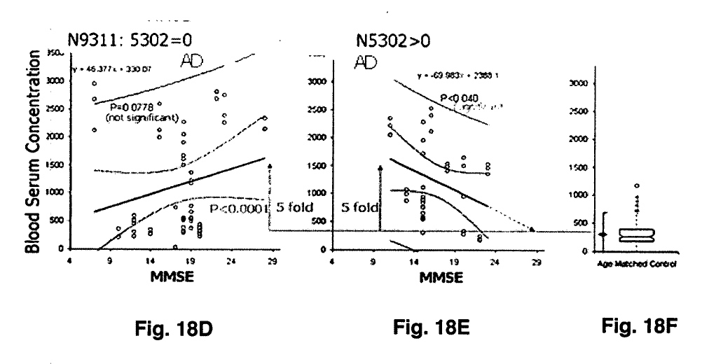

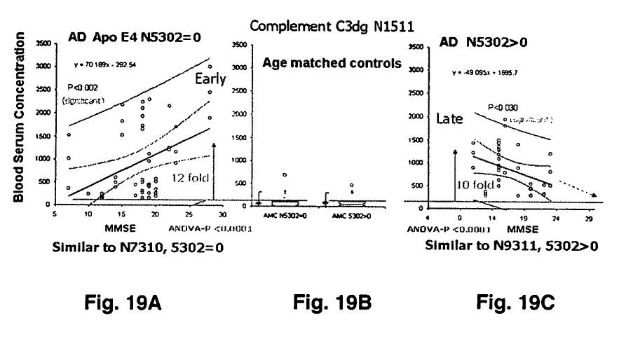

When the severity of Alzheimer's disease is taken into account (MMSE scores) (FIGS. 18, 19), differences in blood serum concentration vs. Alzheimer's disease severity were found between Complement C3c1 protein spot N7310 and Complement C3c2a protein spot N9311, and between the detection, or the lack of detection, of Apolipoprotein E4 protein spot N5302.

In patients with no detectable levels of Apolipoprotein E4 protein spot N5302, blood serum concentration of Complement C3c1 protein spot N7310 is 14 fold higher than age matched normal controls (FIG. 18A, N5302=0, ANOVA-P<0.0001, vs. FIG. 18C). Furthermore, that level declines in a statistically significant fashion with increasing of AD severity (FIG. 18A, Decreasing MMSE=Increasing severity of dementia, Linear Regr.-P<0.0001).

Similarly, in patients with no detectable levels of Apolipoprotein E4 protein spot N5302, (FIG. 19A; N5302=0) expression of Complement C3dg protein spot N1511 in blood serum is 12 fold higher than age matched normal controls (FIG. 19A, N5302=0, ANOVA-P<0.0001, vs. FIG. 19B) and the level declines in a statistically significant fashion with increasing of AD severity (FIG. 19a, Decreasing MMSE=Increasing severity of dementia, Linear Regr.-P<0.002).

On the other hand, in patients with no detectable Apolipoprotein E4 protein spot N5302, the expression of Complement C3c2a protein spot N9311 is higher (5 fold) than age matched normal controls (FIG. 18D, N5302=0, ANOVA-P<0.0001 vs. 18f), but there is no statistically significant correlation in expression levels of N9311 with increasing of AD severity (FIG. 18D, N5302=0, Linear Regr.-P>0.080).

In patients with detectable Apolipoprotein E4 protein spot N5302, expression of Complement C3c1 protein spot N7310 is also higher (5 fold) than age matched normal controls (FIG. 18B, N5302>0, ANOVA-P<0.0001, vs. FIG. 18C), but there is no statistically significant correlation in expression levels of N7310 with increasing AD severity (FIG. 19A, Decreasing MMSE=Increasing severity of dementia, Linear Regr.-P>0.80).

However, expression of Complement C3c2a protein spot N9311 is not significantly higher than age matched controls in mild AD (FIG. 18E, N5302>0, vs. FIG. 18F) but in moderate and severe AD, the levels are 5 fold higher than age matched normal control and is in a significant correlation with increasing of AD severity in patients with detectable Apolipoprotein E4 protein spot N5302, (FIG. 18E, N5302>0, ANOVA-P<0.0001, Linear Regr.-P<0.040, vs. FIG. 18F).

Similar to Complement C3c2a protein spot N9311, in patients with detectable Apolipoprotein E4 protein spot N5302, expression of Complement C3dg protein spot N1511 is not significantly higher than age matched controls in mild AD (FIG. 19C, N5302>0, vs. FIG. 19B) but the levels are 12 fold higher in moderate and severe AD in a statistically significant correlation with increasing AD severity (FIG. 19C, N5302>0, ANOVA-P<0.0001, Linear Regr.-P<0.030, vs. FIG. 19B).

In a preferred embodiment of the invention, Complement C3c1 protein N7310 blood serum concentration significantly above age matched normal controls is an indication for:
1. Early detection of AD and monitoring of AD severity, in patients with no detectable Apolipoprotein E4 protein spot N5302 (N5302=0), and for
2. Early detection of AD but not for monitoring of AD severity, in patients with detectable Apolipoprotein E4 protein spot N5302 (N5302>0).

Also in the preferred embodiment of the invention, concentrations of Complement C3c2a protein spot N9311 significantly above the level of age matched normal controls is an indication for:
3. Early detection of AD but not for monitoring of AD severity, in patients with no detectable Apolipoprotein E4 protein spot N5302 (N5302=0), and for
4. Monitoring of AD severity but not for early detection of AD, in patients with detectable Apolipoprotein E4 protein spot N5302 (N5302>0).

Also in the preferred embodiment of the invention, the effect of detection, or a lack of detection of Apolipoprotein E4 protein spot N5302 expression, as measured in blood serum (whether Apolipoprotein E4 protein spot N5302 concentration is >0 or =0), in association with the expression of Complement C3dg protein spot N1511 is determined. Complement C3dg protein spot N1511 (Table 12, SEQ ID NO. 10) consists of a different amino acid sequence derived from a sequence domain downstream of the locus shared by Complement C3c1 protein spot N7310 and C3c2a protein spot N9311, of Complement C3 (Table 8, SEQ ID NO. 6) parent precursor and also derived from as an alternative isoform (Table 13, SEQ ID NO. 11) derived from an alternate parent precursor Similar to C3 (Table 11, SEQ ID NO. 9).

Thus, in a preferred embodiment of the invention, the significantly higher level of blood serum concentration of Complement C3dg protein spot N1511 in Alzheimer's disease patients than that of aged matched normal controls is an indication for:
1. Early detection of AD and monitoring of AD severity, in patients with no detectable Apolipoprotein E4 protein spot N5302 (N5302=0; decreasing blood serum concentration of Complement C3dg protein spot N1511 with increasing Alzheimer's disease severity), and for
2. Monitoring of AD severity but not for early detection of AD, in patients with detectable Apolipoprotein E4 protein spot N5302 (N5302>0; increasing blood serum concentration of Complement C3dg protein spot N1511 with increasing Alzheimer's disease severity).

### The Haptoglobin HP-1 Proteins N1514, N2401, N2407, and N3409

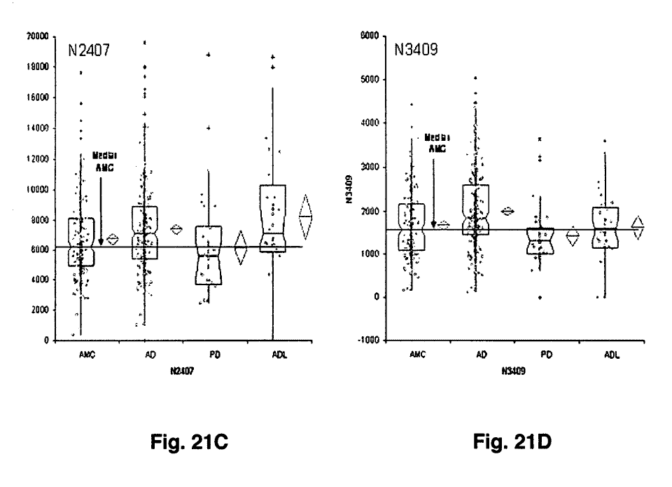

Haptoglobin HP-1 Protein spots N1514, N2401, N2407, and N3409 contain the same amino acid sequence (SEQ ID NO. 19), but differ in their electrophoretic mobility in 2D gel electrophoresis (FIG. 2). They are up-regulated in parallel in the blood serum of patients with Alzheimer's disease and patients with AD-like and mixed dementias, but not in patients with Parkinson's disease, as compared to age matched normal controls (FIG. 21).

As shown in FIG. 21D, Differential expression of Haptoglobin HP-1 protein spot N3409 between Alzheimer's disease patients and Parkinson's disease patients is particularly pronounced. In Alzheimer's disease patients, Haptoglobin HP-1 protein spot N3409 is up-regulated from age matched normal controls, whereas in Parkinson's disease patients, Haptoglobin HP-1 protein spot N3409 is down regulated from age matched normal controls. This provides for significantly higher sensitivity and specificity for distinguishing between these two diseases based on the concentration of Haptoglobin HP-1 protein spot N3409 (71.9% and 72.8%, respectively, FIG. 21E)

In a preferred embodiment of the invention, the concentrations of Haptoglobin HP-1 protein spots N1514, N2401, N2407, and N3409 and their sum (HP-1 Total Proteins, FIG. 22) are employed. HP-1 Total Proteins are up-regulated in a statistically significant manner in the blood serum of patients with Alzheimer's disease, and patients with AD-like and Mixed dementias, but not in patients with Parkinson's disease, as compared to age matched normal controls (FIG. 22A, ANOVA-P<0.0030).

Using the ROC analysis, the Total of HP-1 Protein spots showed sensitivities and specificities of discrimination between 115 Alzheimer's disease patients and 75 age matched normal control individuals as follows:

HP-1 Total Proteins: 56.2% sensitivity, 56.0% specificity (Table 18, AD>30136 ppm, ROC-P<0.0001).

Furthermore, in Alzheimer's disease patients with no detectable Apolipoprotein E4 protein spot N5302 (N5302=0), blood serum concentrations of Haptoglobin HP-1 protein spots N1514, N2401, N2407, N3409, and HP-1 Total Proteins are significantly higher than age matched normal controls (FIG. 23, FIG. 25A, N5302=0, ANOVA-P<0.0001).

On the other hand, in Alzheimer's disease patients and age matched normal controls with detectable blood serum levels of Apolipoprotein E4 protein spot N5302 (N5302>0), Haptoglobin HP-1 Proteins spots N1514, N2401, N2407, N3409, and HP-1 Total Proteins are not significantly different from the levels of age matched controls (FIG. 23, FIG. 25A, N5302=0, ANOVA-P>0.7). However, the concentrations of Haptoglobin HP-1 Proteins N1514, N2401, N2407, N3409, and HP-1 Total Proteins in Alzheimer's disease patients and age matched normal controls with detectable levels of Apolipoprotein E4 protein spot N5302 (N5302>0 are both significantly higher than age matched normal controls with no detectable levels of Apolipoprotein E4 protein N5302 (N5302=0) (FIGS. 23-25).

ROC analysis demonstrated specificity and sensitivity for separation between Alzheimer's disease patients and age matched normal controls with no detectable Apolipoprotein E4 protein spot N5302, and no specificity nor sensitivity for separation between Alzheimer's disease patients and age matched normal controls with detectable Apolipoprotein E4 protein spot N5302 (FIG. 25b) as follows:
1. HP-1 Total Proteins: N5302=0, 64.6% sensitivity, 64.7% specificity (Table 19, AD>30216 ppm, ROC-P<0.0001);
2. HP-1 Total Proteins: N5302>0, 44.8% sensitivity, 44.9% specificity (Table 19, AD>30216 ppm, ROC-P<0.0001);

Thus, in a preferred embodiment of the invention, the significantly higher level of blood serum concentration of Haptoglobin HP-1 Protein spots N1514, N2401, N2407, N3409, and HP-1 Total Proteins (N1514+N2401+N2407+N3409) in Alzheimer's disease patients than that of aged matched normal controls is an indication for:
3. Detection of AD in patients with no detectable Apolipoprotein E4 protein spot N5302 (N5302=0), and
4. Discrimination of patients with AD from patients with PD.
5. But not for detection of AD in patients with detectable levels of Apolipoprotein E4 protein spot N5302 (N5302>0), Inter-alpha-trypsin inhibitor heavy chain H4 related 35 KD protein spot N2307

As shown in FIG. 26 and Table 20, Inter alpha trypsin inhibitor heavy chain H4 related 35 KD protein spot N2307 is significantly up-regulated in the blood serum of patients with Alzheimer's disease (ANOVA-P<0.0001), Parkinson's disease, and with Stroke related, and Mixed dementias, and conversely was significantly down regulated in blood serum of patients with non-stroke related dementias, including: Frontotemporal dementia, Lewy body dementia, Corticalbasal Ganglionic degeneration, alcohol related dementia, and semantic dementia, as compared to age matched normal controls (FIG. 26A, Table 20).

Using ROC analysis (FIG. 26B, Table 20), the blood serum concentration of Inter alpha trypsin inhibitor heavy chain H4 related 35 KD protein spot N2307 distinguishes between 115 patients with Alzheimer's disease and 75 age matched normal controls (FIG. 26B) as follows: N2307: 58.0% sensitivity, 58.2% specificity (Table 20, AD>210 ppm, ROC-P<0.0001).

In Alzheimer's disease patients and age matched controls with and without detectable blood serum levels of Apolipoprotein E4 protein spot N5302, the expression levels of Inter alpha trypsin inhibitor heavy chain H4 related 35 KD protein spot N2307 in blood serum is significantly higher than complementary age matched normal controls (FIG. 27A, N5302=0, Table 21a, ANOVA-P<0.0001; ANOVA-P>0.06).

Using an ROC analysis (FIG. 27B, Table 21b), blood serum concentration of Inter alpha trypsin inhibitor heavy chain H4 related 35 KD protein spot N2307 demonstrated sensitivity and specificity for diagnosis of AD from age matched normal controls when Apolipoprotein E4 protein spot N5302 was not detected (N5302=0) but not when Apolipoprotein E4 protein spot N5302 was detected (N5302>0) in blood serum:
1. N2307: N5302=0; 61.8% sensitivity, 61.5% specificity (Table 21b, AD>211 ppm, ROC-P<0.0001);
2. N2307: N5302>0, 50.7% sensitivity, 50.7% specificity (Table 21b, AD>224 ppm, ROC-P>0.14).

Furthermore, as shown in FIG. 28, expression of Inter alpha trypsin inhibitor heavy chain H4 related 35 KD protein spot N2307 in blood serum is significantly higher (2.7 fold) than the age matched normal controls in mild Alzheimer's disease patients without detectable blood serum level of Apolipoprotein E4 protein spot N5302 (FIG. 28A, N5302=0; ANOVA-P<0.0001 vs. FIG. 28B), in this group of patients, there is statistically significant decline in expression levels of Inter alpha trypsin inhibitor heavy chain H4 related 35 KD protein spot N2307, in correlation with increasing the severity of AD. These results are similar to that of Complement C3c1 protein spot N7310 (FIG. 28A Linear Regr-P vs. FIG. 28B, compare with FIG. 18A, N7310, N5302=0).

Also, as shown in FIG. 28, expression of Inter alpha trypsin inhibitor heavy chain H4 related 35 KD protein spot N2307 in blood serum is also significantly higher (2.2 fold) than the age matched normal controls in mild Alzheimer's disease in patients with detectable Apolipoprotein E4 protein spot N5302. In this group of patients, there is no significant correlation with the increased severity of AD (FIG. 28C, N5302>0 vs. ANOVA-P<0.0001, Linear Regr.-P>0.20 vs. FIG. 28B). These results are also similar to that of Complement C3c1 protein N7310 (compare with FIG. 18B, N5302>0).

Thus in a preferred embodiment of the invention, as in the case of Complement C3c1 protein N7310, the significantly higher level of the blood serum concentrations of Inter alpha trypsin inhibitor heavy chain H4 related 35 KD protein spot N2307, in Alzheimer's disease patients than that of the age matched normal controls, is an indication for:
1. Early detection of AD and monitoring of AD severity, in patients with no detectable Apolipoprotein E4 protein spot N5302 (N5302=0), and for
2. Early detection of AD but not for monitoring of AD severity, in patients with detectable Apolipoprotein E4 spot protein N5302 (N5302>0); Immunoglobulin Light Chain Protein N6224 and Apolipoprotein A-IV Protein spot N2502

As shown in FIGS. 29 and 31, Tables 22 and 24, Immunoglobulin Light Chain protein spot N6224 and Apolipoprotein A-IV protein spot N2502 are both significantly down-regulated in the blood serum of patients with Alzheimer's disease, Parkinson's disease, and AD-Like and Mixed dementias, as compared to age matched normal controls (FIG. 29A, Table 22, N6224, ANOVA-P<0.0002; FIG. 31A, Table 24, N2502, ANOVA-P<0.0001).

Using the ROC analysis (FIG. 29B, Table 22, FIG. 31B, Table 24), the blood serum concentrations of both Immunoglobulin Light Chain protein spot N6224 and Apolipoprotein A-IV protein spot N2502 distinguish between 115 patients with Alzheimer's disease and 75 age matched normal controls (FIG. 29B, N6224, FIG. 31B, N2502) as follows:
1. N6224: 59.7% sensitivity, 59.6% specificity (Table 22b, AD<368 ppm, ROC-P<0.0001).
2. N2502: 59.1% sensitivity, 59.1% specificity (Table 23b, AD<2465 ppm, ROC-P<0.0001).

Down-regulated blood serum levels of Immunoglobulin Light Chain protein spot N6224 and Apolipoprotein A-IV Protein spot N2502 in Alzheimer's disease patients below that of age matched normal controls are found regardless of whether Apolipoprotein E4 protein spot N5302 was detected or not in blood serum; although more significant in the case of N5302>0 for Immunoglobulin Light Chain protein spot N6224 (FIG. 30A, N6224, Table 23, N5302=0, ANOVA-P>0.08; N5302>0, ANOVA-P<0.0007), and more significant in the case of N5302=0 for Apolipoprotein A-IV protein spot N2502 (FIG. 32A, N2502, Table 25, N5302=0, ANOVA-P<0.0003; N5302>0, ANOVA-P<0.03).

Using the ROC analysis (FIG. 30B, Table 23, FIG. 32B, Table 25), blood serum levels of Immunoglobulin Light Chain protein spot N6224 and Apolipoprotein A-IV protein spot N2502 distinguish between Alzheimer's disease patients and age matched normal controls whether Apolipoprotein E4 protein spot N5302 was detected or not in blood serum as follows:
N6224: N5302=0; 56.9% sensitivity, 57.1% specificity (Table 23, AD<368 ppm, ROC-P<0.0003);
N6224: N5302>0, 62.7% sensitivity, 62.3% specificity (Table 23, AD<378 ppm, ROC-P<0.0001).
N2502: N5302=0; 58.3% sensitivity, 58.3% specificity (Table 25, AD<2412 ppm, ROC-P<0.0001);
N2502: N5302>0; 62.2% sensitivity, 62.3% specificity (Table 25, AD<2588 ppm, ROC-P<0.0003);

Thus in a preferred embodiment of the invention, the significantly low blood serum levels of Immunoglobulin Light Chain protein spot N6224 and Apolipoprotein A-IV protein spot N2502 in Alzheimer's disease patients than that of the age matched normal controls is an indication for: Detection of AD in patients with no detectable Apolipoprotein E4 protein spot N5302 (N5302=0), and for Detection of AD in patients with detectable Apolipoprotein E4 protein spot N5302 (N5302>0);

As illustrated in Table 26, in a preferred embodiment of the invention, when the blood serum concentrations of Apolipoprotein E4 protein spot N5302 and Apolipoprotein E3 protein spot N3314, Complement Factor H/Hs protein spot N4411, Complement Factor Bb protein spot N7616, Complement C3c1 phosphoprotein spot N7310, Complement C3c2a protein spot N9311, Complement C3dg protein spot N1511, Haptoglobin HP-1 Total Proteins (N1514+N2401+N2407+N3409), Inter alpha trypsin inhibitor heavy chain (H4) related 35 KD protein spot N2307, Immunoglobulin Light Chain Protein spot N6224 and Apolipoprotein A-IV protein spot N2502 are all combined into a multivariate linear discriminant function to distinguish between all 115 Alzheimer's disease patients and all 75 age matched normal controls, a sensitivity of 69.6% and a specificity of 84.4% are obtained. However, when the Alzheimer's disease patients and age-matched normal control subjects are separated into two groups, based on whether Apolipoprotein E4 protein spot N5302 is detected or not in the blood serum, a sensitivity of 82.3% and a specificity of 82.7% are obtained when the results are combined after the discriminant analysis (Table 26). These results underscore the importance of differentiation between two types of Alzheimer's disease patients for the purpose of better sensitivity during diagnosis of the disease.

In a preferred embodiment of the invention separate linear discriminant functions are performed for those in whom Apolipoprotein E4 protein spot N5302 is detected in blood serum (N5302>0) and those in whom Apolipoprotein E4 protein spot N5302 is not detected in blood serum (N5302=0). In each linear discriminant function, Alzheimer's disease patients and Age matched normal controls are distinguished from one another. Also in the preferred embodiment of the invention, the linear discriminant function is generated with the addition of concentrations of other blood serum protein biomarkers, for example, one or more of Apolipoprotein E3 protein spot N3314, Complement Factor H/Hs protein spot N4411, Complement Factor Bb protein spot N7616, Complement C3c1 phosphoprotein spot N7310, Complement C3c2a protein spot N9311, Complement C3dg protein spot N1511, Haptoglobin HP-1 individual and Total of protein spots (N1514+N2401+N2407+N3409), Inter alpha trypsin inhibitor heavy chain (H4) related 35 KD protein spot N2307, Immunoglobulin Light Chain protein spot N6224 and Apolipoprotein A-IV protein spot N2502.

When separate discriminant functions are performed in the manner of the invention (Table 26) and the results are then combined by adding the true positives together, the true negatives together, the false positives together, and the false negatives together, that were generated by the separate discriminant functions, this results in clinically significant sensitivity and specificity (Table 26, Sensitivity 82.3%, Specificity 82.7%).

Each step of sensitivity and specificity improvements for diagnosis of Alzheimer's disease attained by the invention are shown in FIG. 33. Furthermore, the invention is built by leveraging individual biomarkers with individual utilities that fall into types as illustrated in FIG. 34 (Types 1-5) based on their relationship to the Alzheimer's disease and the ways in which the disease manifests.

FIGS. 35-36 illustrate the disease pathways indicated by the abnormal changes in concentration of some of these blood serum protein biomarkers: Apolipoprotein E3 (Apo E3); Transthyretin Dimer (TTD); Inter-alpha-trypsin inhibitor heavy chain (H4) related 35 KD protein (ITI(H4)RP 35 KD); Complement C3c1 tyrosine phosphoprotein (C3c1(p)); Complement C3c2a protein (C3c2a); Complement C3dg protein (C3dg); Complement Factor H protein (Factor H), and Complement Factor Bb protein (Factor Bb); all of which have been disclosed before in connection with inventions for diagnosis and monitoring of neurodegenerative diseases (refs. 17-19; U.S. Utility patent application Ser. No. 11/507,337 filed Aug. 22, 2006 and entitled “Assay for Diagnosis and Therapeutics Employing Similarities and Differences in Blood Serum Concentrations of 3 forms of Complement C3c and Related Protein Biomarkers between Amyotrophic Lateral Sclerosis and Parkinson's Disease” by inventors Ira L. Goldknopf et al., U.S. Provisional Patent Application Ser. No. 60/901,467 filed Feb. 16, 2007 and entitled “Forty Seven (47) Protein Biomarkers for Neurodegenerative Diseases,” by inventors Ira L. Goldknopf et al., U.S. Utility patent application Ser. No. 12/069,807 filed Feb. 14, 2008 and entitled “Forty Seven (47) Protein Biomarkers for Neurodegenerative Diseases,” by inventors Ira L. Goldknopf, U.S. Utility patent application Ser. No. 11/602,814 filed 11/21/06 and entitled “An Inter-Alpha Trypsin Inhibitor Heavy Chain (H4) Related Protein as a Biomarker of Alzheimer's Disease,” by inventors Ira L. Goldknopf, et al, U.S. Utility patent application Ser. No. pending filed Aug. 29, 2007 and entitled “A Complement Factor H Protein as a Biomarker of Parkinson's Disease,” by inventors Ira L. Goldknopf, et al., U.S. Utility patent application Ser. No. pending filed Sep. 5, 2007 and entitled “An Apolipoprotein E3 Protein as a Biomarker of Parkinson's Disease,” by inventors Ira L. Goldknopf, et al., and herein all incorporated by reference).

In this preferred embodiment of the invention we have compared these changes as a function of the detection (FIG. 35A, B; FIG. 36B) or lack of detection (FIG. 35C, D; FIG. 36A) of Apolipoprotein E4 protein in the blood serum of the patients. We have found parallel, specific differences between Alzheimer's disease patients and age matched normal controls in the blood serum concentrations of two biomarkers that are closely related to the Apo E ε4 gene allele protein product, Apolipoprotein E4 protein spot N5302: Apo E ε3 gene allele protein product, Apolipoprotein E3 protein spot N3314; and Transthyretin “Dimer” protein spot N3307.

When both the AD patients and controls had detectable blood serum levels of Apolipoprotein E4 protein spot N5302, the protein biomarkers Apolipoprotein E3 protein spot N3314 and Transthyretin “Dimer” protein spot N3307 were markedly reduced in blood serum concentration in the Alzheimer's disease patients (FIG. 34 Type 1, FIG. 35A, FIG. 36B; N5302>0). However, the reductions in blood serum concentrations of these two protein biomarkers were much less pronounced when both AD patients and controls had no detectable blood serum levels of Apolipoprotein E4 protein (FIG. 34 Type 1, FIG. 35C, FIG. 36A, N5302=0).

Those patients for which the Apolipoprotein E4 protein spot N5302 was detected in their blood serum (Apo E4>0, FIG. 35A, B, FIG. 36B) must have at least 1 copy of the Apo E ε4 gene allele in their genome, and the allele is expressed as a protein (spot N5302). To uncover the significance of these differences, we incorporate the well established findings that persons who carry the gene allele Apo E ε4 have substantially higher risk of developing Alzheimer's disease and other dementias (6, 38-39), and also have higher levels of the Amyloid plaque forming Aβ-42 and 1-40 peptides, higher levels of the Amyloid plaques, of the neurofibrillary tangle forming hyper-phosphorylated Tau, and of the neurofibrillary tangles than individuals without the Apo E ε4 allele. All of these data correlate with the development of AD. Furthermore, these differences are reflected in normal controls and even greater in Alzheimer's disease patients (40-42).

Neuronal Degeneration in Alzheimer's Disease Patients with Detectable Apolipoprotein E4 Protein in Blood Serum

The marked reduction in the blood serum concentration of soluble Apolipoprotein E3 protein spot N3314 and Transthyretin “Dimer” protein spot N3307, in patients with detectable blood serum Apolipoprotein E4 protein spot N5302 (FIG. 35A, FIG. 36B, N5302>0) is attributable to their known incorporation into insoluble Amyloid plaques and neurofibrillary tangles (17, 18, 43), which are also known to be increased in Apo E ε4 gene allele positive AD patients (40-42). The reduced level (46%, compared to AMC) of soluble Apolipoprotein E3 protein in this type of patients would also attenuate the known neuro-protective mechanisms against oxidative stress that are also known to be facilitated by soluble Apolipoprotein E3. These include: 1) maintenance of intra-neuronal cholesterol; and metabolism of peroxidized lipids; both mediated by the Apo E receptor; and NMDA receptor mediated glutamate/calcium homeostasis, (6, 17-19, 40-46; FIGS. 35A, 36B, 37B).

Such diminished neuroprotection, is coincident with known markedly increased oxidative stress in Apo E ε4 allele positive AD (47-52), resulting in uncontrolled neuronal oxidative stress and apoptosis as the primary neurodegenerative pathway driving AD in Apolipoprotein E4 protein spot N5302 positive patients (FIGS. 35A; 36B).

In Apolipoprotein E4 protein spot N5302 positive patients (N5302>0), we have also found elevated blood serum levels of other protein biomarkers (FIGS. 34, 35B; 36B) indicative of a secondary neurodegenerative pathway of inflammation. Two of these proteins were previously found associated with localized acquired auto-immune inflammation in sporadic ALS and Parkinson's disease (17-19, and U.S. Utility patent application Ser. No. 11/507,337 filed Aug. 22, 2006 and entitled “Assay for Diagnosis and Therapeutics Employing Similarities and Differences in Blood Serum Concentrations of 3 forms of Complement C3c and Related Protein Biomarkers between Amyotrophic Lateral Sclerosis and Parkinson's Disease” by inventors Ira L. Goldknopf et al. and herein incorporated by reference). This included elevated blood serum levels of Complement C3c1 tyrosine phosphoprotein spot N7310 and Complement Factor Bb protein spot N7616. In addition, in Apolipoprotein E4 protein spot N5302 positive Alzheimer's disease patients, there were delayed elevations of blood serum concentrations, i.e. in severe AD, of Complement C3c2a protein (C3c2a) spot N9311; Complement C3dg protein (C3dg) spot N1511; and Inter-alpha-trypsin inhibitor heavy chain (H4) related 35 KD protein (ITI (H4) RP 35 KD) spot N2307, known systemic innate inflammatory response associated proteins (Ref. 17-19). In AD, this secondary innate inflammatory pathway is most likely due to the known enhanced induction and secretion of inflammatory cytokines, particularly IL-6 in response to increased Amyloidosis and neurofibrillary tangles in Apo E ε4 positive AD (FIGS. 35B, 36B; refs. 21-25, 31-37, 53, 54).

Thus in the preferred embodiment of the invention, in Alzheimer's disease patients with detectable Apolipoprotein E4 protein spot N5302 in their blood serum (FIG. 35 A, B; N5302>0), the predominant or primary mechanism driving neurodegeneration is Aβ/NFT-induced oxidative stress leading to neuronal apoptosis, with a secondary immune inflammatory response due to delayed Aβ/NFT-induced pro-inflammatory cytokine induction.

Neuronal Degeneration in Alzheimer's Disease Patients with No Detectable Apolipoprotein E4 Protein in Blood Serum

A different pattern emerged when AD patients with no detectable Apolipoprotein E4 protein spot N5302 in their blood serum were compared to a group of normal controls, also with no detectable Apolipoprotein E4 protein spot N5302 in their blood serum (FIG. 35 C, D; N5302=0). In these AD patients, there was little reduction (20%, compared to AMC) in blood serum concentration of Apolipoprotein E3 protein spot N3314, leaving intact the neuroprotective maintenance of cholesterol homeostasis and attenuation of oxidative stress, known to be associated with increased concentration of Apolipoprotein E (6, 17-19, 40-46, 55-59). It is also well known that accumulation of Aβ and NFTs, and the concomitant generation of oxidative stress intermediates is much less in AD patients lacking the Apo E ε4 allele than in those that have the allele (40-42, 47-51). This combination of factors should markedly attenuate the oxidative stress related apoptosis and the Aβ/NFT-induced pro-inflammatory cytokine induction of inflammation demonstrated in Apolipoprotein E4 protein spot N5302 positive Alzheimer's patients (FIG. 35B).

Nevertheless, Apolipoprotein E4 protein spot N5302 positive Alzheimer's disease patients are also undergoing neurodegeneration. The answer lies in the additional biomarkers of acquired immune and innate inflammation (FIG. 1D). These include a pattern of pronounced elevation in the blood serum concentration of: Complement C3c1 phosphoprotein spot N7310 (8, 9, 38-40); paralleled by similar elevations in Complement C3dg protein spot N1511 and Inter-a-trypsin inhibitor heavy chain H4 related 35 KD protein spot N2307; all three of which are at maximally high levels in mild AD, somewhat less high in moderate AD, and slightly high in severe AD patients' blood serum (FIG. 35D; N5302=0).

Also the blood serum levels of innate immune inflammatory biomarkers (8, 9, 38-40) Complement C3c2a protein spot N9311, Complement Factor H/Hs protein spot N4411 and Complement Factor Bb protein spot N7616 were all elevated to moderately high levels in mild, moderate and severe AD.

Thus, in the preferred embodiment of the invention, in the Alzheimer's disease patients with no detectable Apolipoprotein E4 protein spot N5302 in their blood serum (N5302=0), the apoptosis pathway is inhibited and auto-immune inflammation is the predominant pathway driving neuronal degeneration in these patients.

Analogies with Other Neurodegenerative Diseases

As illustrated in Table 27, our findings with blood serum biomarkers in Alzheimer's disease were analogous to our previous findings with the same blood serum protein biomarkers in ALS (17, 18): familial ALS resembles Apolipoprotein E4 protein spot N5302 positive Alzheimer's disease; and sporadic ALS resembles Apolipoprotein E4 protein spot N5302 negative Alzheimer's disease. Thus the expression of an Apo E ε4 allele protein (N5302>0; a single amino acid mutation in 14% of the population), which signifies higher risk of Alzheimer's disease (5, 6, 38, 39), as well as cognitive deficits in “normal” aged individuals (38), leads to a primary oxidative stress driven apoptotic Alzheimer's disease phenotype, just as does the expression of the ALS risk genetic mutant Superoxide dismutase protein in familial ALS (17, 18) Similarly, in Alzheimer's disease patients not expressing the Apo E ε4 allele protein (N5302=0), an immune inflammatory mechanism is responsible for driving neurodegeneration, just as is the case in the absence of the Superoxide dismutase mutations in sporatic ALS (17, 18) and in Parkinson's disease (17, 18, 63).

### Applications for Similarities and Differences in Neurodegenerative Diseases

Proteins in the blood serum can tell us what disease pathways and mechanisms of neuronal degeneration are active in the patients. We have illustrated this with mechanistic differences, as indicated by blood serum proteomics, between two different types of Alzheimer's disease, and previously between two different types of ALS (17, 18). The mechanisms of neurodegeneration that display variations between two forms of each disease are oxidative stress, apoptosis, and immune inflammatory phagocytosis. These familial vs. sporadic disease variations in mechanisms are demonstrated both by Alzheimer's disease and ALS (Table 28). However, when additional blood serum proteins are brought into the analysis, disease specific differences emerge, with capabilities for differential diagnosis between diseases with similar symptoms (Table 29, ref. 19), implying additional disease specific mechanistic differences, which will ultimately lead to differential treatment and personalized medicine (Table 28, ref. 19).

### Additional Embodiments

The blood serum samples may also be subjected to various other techniques known in the art for separating and quantitating proteins. Such techniques include, but are not limited to: gel filtration chromatography, ion exchange chromatography, reverse phase chromatography, affinity chromatography (typically in an HPLC or FPLC apparatus), affinity capture, one dimensional gel or capillary electrophoresis, or any of the various centrifugation techniques well known in the art. Certain embodiments would also include a combination of one or more chromatography; electrophoresis or centrifugation steps combined via electrospray or nanospray with mass spectrometry or tandem mass spectrometry of the proteins themselves, or of a total digest of the protein mixtures. Certain embodiments may also include surface enhanced laser desorption mass spectrometry or tandem mass spectrometry, or any protein separation technique that determines the pattern of proteins in the mixture, either as a one-dimensional, two-dimensional, three-dimensional or multi-dimensional protein pattern, and/or the pattern of protein post synthetic modifications or different isoforms of an Apolipoprotein E4 protein, an Apolipoprotein E3 protein, a Transthyretin protein, a Complement C3c1 protein, a Complement C3c2a protein, a Complement C3dg protein, a Complement Factor Bb protein, A Complement Factor H protein, and/or an Inter-alpha Trypsin Inhibitor protein, are used.

Quantitation of a protein by antibodies directed against that protein is well known in the field. The techniques and methodologies for the production of one or more antibodies to an Apolipoprotein E4 protein, an Apolipoprotein E3 protein, a Transthyretin protein, a Complement C3c1 protein, a Complement C3c2a protein, a Complement C3dg protein, a Complement Factor Bb protein, A Complement Factor H protein, and/or an Inter-alpha Trypsin Inhibitor Heavy Chain (H4) related protein, are routine in the field and are not described in detail herein.

As used herein, the term antibody is intended to refer broadly to any immunologic binding agent such as IgG, IgM, IgA, IgD and IgE. Generally, IgG and/or IgM are preferred because they are the most common antibodies in the physiological situation and because they are most easily made in a laboratory setting.

Monoclonal antibodies (MAbs) are recognized to have certain advantages, e.g., reproducibility and large-scale production, and their use is generally preferred. The invention thus provides monoclonal antibodies of human, murine, monkey, rat, hamster, rabbit, chicken, or other animal origin. Due to the ease of preparation and ready availability of reagents, murine monoclonal antibodies are generally preferred. However, human auto antibodies or “humanized” antibodies are also contemplated, as are chimeric antibodies from mouse, rat, or other species, bearing human constant and/or variable region domains, bispecific antibodies, recombinant and engineered antibodies and fragments thereof.

The term “antibody” thus also refers to any antibody-like molecule that has a 20 amino acid antigen binding region, and includes antibody fragments such as Fab′, Fab, F(ab′)2, single domain antibodies (DABS), Fv, scFv (single chain Fv), and the like. The techniques for preparing and using various antibody-based constructs and fragments are well known in the art. Means of preparing and characterizing antibodies are also well known in the art (See, e.g., Antibodies: A Laboratory Manual, Cold Spring Harbor Laboratory, 1988; incorporated herein by reference).

Antibodies to an Apolipoprotein E4 protein, an Apolipoprotein E3 protein, a Transthyretin protein, a Complement C3c1 protein, a Complement C3c2a protein, a Complement C3dg protein, a Complement Factor Bb protein, A Complement Factor H protein, and/or an Inter-alpha Trypsin Inhibitor Heavy Chain (H4) related protein may be used in a variety of assays in order to quantitate the protein in serum samples, or other fluid or tissue samples. Well known methods include immunoprecipitation, antibody sandwich assays, ELISA and affinity chromatography methods that include antibodies bound to a solid support. Such methods also include micro arrays of antibodies or proteins contained on a glass slide or a silicon chip, for example.

It is contemplated that arrays of antibodies to an Apolipoprotein E3 protein, or peptides derived from an Apolipoprotein E4 protein, an Apolipoprotein E3 protein, a Transthyretin protein, a Complement C3c1 protein, a Complement C3c2a protein, a Complement C3dg protein, a Complement Factor Bb protein, A Complement Factor H protein, and/or an Inter-alpha Trypsin Inhibitor Heavy Chain (H4) related protein, may be produced in an array and contacted with the serum samples or protein fractions of serum samples in order to quantitate the blood serum concentrations of an Apolipoprotein E4 protein, an Apolipoprotein E3 protein, a Transthyretin protein, a Complement C3c1 protein, a Complement C3c2a protein, a Complement C3dg protein, a Complement Factor Bb protein, A Complement Factor H protein, and/or an Inter-alpha Trypsin Inhibitor Heavy Chain (H4) related protein. The use of such micro arrays is well known in the art and is described, for example in U.S. Pat. No. 5,143,854 incorporated herein by reference.

The present invention includes a screening assay for neurodegenerative disease based on the up-regulation and/or down-regulation of an Apolipoprotein E4 protein, an Apolipoprotein E3 protein, a Transthyretin protein, a Complement C3c1 protein, a Complement C3c2a protein, a Complement C3dg protein, a Complement Factor Bb protein, A Complement Factor H protein, and/or an Inter-alpha Trypsin Inhibitor Heavy Chain (H4) related protein expression. One embodiment of the assay will be constructed with antibodies to an Apolipoprotein E4 protein, an Apolipoprotein E3 protein, a Transthyretin protein, a Complement C3c1 protein, a Complement C3c2a protein, a Complement C3dg protein, a Complement Factor Bb protein, A Complement Factor H protein, and/or an Inter-alpha Trypsin Inhibitor Heavy Chain (H4) related protein. One or more antibodies targeted to antigenic determinants of an Apolipoprotein E4 protein, an Apolipoprotein E3 protein, a Transthyretin protein, a Complement C3c1 protein, a Complement C3c2a protein, a Complement C3dg protein, a Complement Factor Bb protein, A Complement Factor H protein, and/or an Inter-alpha Trypsin Inhibitor Heavy Chain (H4) related protein, will be spotted onto a surface, such as a polyvinyl membrane or glass slide. As the antibodies used will each recognize an antigenic determinant of an Apolipoprotein E4 protein, an Apolipoprotein E3 protein, a Transthyretin protein, a Complement C3c1 protein, a Complement C3c2a protein, a Complement C3dg protein, a Complement Factor Bb protein, A Complement Factor H protein, and/or an Inter-alpha Trypsin Inhibitor Heavy Chain (H4) related protein, incubation of the spots with patient samples will permit attachment of an Apolipoprotein E4 protein, an Apolipoprotein E3 protein, a Transthyretin protein, a Complement C3c1 protein, a Complement C3c2a protein, a Complement C3dg protein, a Complement Factor Bb protein, A Complement Factor H protein, and/or an Inter-alpha Trypsin Inhibitor Heavy Chain (H4) related protein, to the antibody.

The binding of an Apolipoprotein E4 protein, an Apolipoprotein E3 protein, a Transthyretin protein, a Complement C3c1 protein, a Complement C3c2a protein, a Complement C3dg protein, a Complement Factor Bb protein, A Complement Factor H protein, and/or an Inter-alpha Trypsin Inhibitor Heavy Chain (H4) related protein, can be reported using any of the known reporter techniques including radioimmunoassay (RIA), stains, enzyme linked immunosorbant assays (ELISA), and sandwich ELISAs with a horseradish peroxidase (HRP)-conjugated second antibody also recognizing an Apolipoprotein E4 protein, an Apolipoprotein E3 protein, a Transthyretin protein, a Complement C3c1 protein, a Complement C3c2a protein, a Complement C3dg protein, a Complement Factor Bb protein, A Complement Factor H protein, and/or an Inter-alpha Trypsin Inhibitor Heavy Chain (H4) related protein, the pre-binding of fluorescent dyes to the proteins in the sample, or biotinylating the proteins in the sample and using an HRP-bound streptavidin reporter. The HRP can be developed with a chemiluminescent, fluorescent, or colorimetric reporter. Other enzymes, such as luciferase or glucose oxidase, or any enzyme that can be used to develop light or color can be utilized at this step.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods, and in the steps or in the sequence of steps of the methods described herein without departing from the concept, spirit and scope of the invention.

More specifically, it is well recognized in the art that the statistical data, including but not limited to the mean, standard error, standard deviation, median, interquartile range, 95% confidence limits, results of analysis of variance, non-parametric median tests, discriminant analysis, etc., will vary as data from additional patients are added to the database or antibodies are utilized to determine concentrations of an Apolipoprotein E4 protein, an Apolipoprotein E3 protein, a Transthyretin protein, a Complement C3c1 protein, a Complement C3c2a protein, a Complement C3dg protein, a Complement Factor Bb protein, A Complement Factor H protein, and/or an Inter-alpha Trypsin Inhibitor Heavy Chain (H4) related protein, or any biomarker. Therefore changes in the range of concentrations of an Apolipoprotein E4 protein, an Apolipoprotein E3 protein, a Transthyretin protein, a Complement C3c1 protein, a Complement C3c2a protein, a Complement C3dg protein, a Complement Factor Bb protein, A Complement Factor H protein, and/or an Inter-alpha Trypsin Inhibitor Heavy Chain (H4) related protein, do not depart from the concept, spirit and scope of the invention.

Also more specifically, it is disclosed (in cross referenced U.S. Utility patent application by Goldknopf, I. L. et al. Ser. Nos. 11/507,337 and 11/503,881, U.S. Provisional Patent Applications by Goldknopf et al. Ser. Nos. 60/708,992 and 60/738,710, and referenced in Goldknopf, I. L. et al. 2006 and E. A. Sheta et al, 2006, hereby incorporated as reference) that blood serum concentrations of protein biomarkers, including an Apolipoprotein E4 protein, an Apolipoprotein E3 protein, a Transthyretin protein, a Complement C3c1 protein, a Complement C3c2a protein, a Complement C3dg protein, a Complement Factor Bb protein, A Complement Factor H protein, and/or an Inter-alpha Trypsin Inhibitor Heavy Chain (H4) related protein, protein spot N3314, can be used in combination with other biomarkers for diagnosis, differential diagnosis, and screening. Consequently, the use of an Apolipoprotein E4 protein, an Apolipoprotein E3 protein, a Transthyretin protein, a Complement C3c1 protein, a Complement C3c2a protein, a Complement C3dg protein, a Complement Factor Bb protein, A Complement Factor H protein, and/or an Inter-alpha Trypsin Inhibitor Heavy Chain (H4) related protein, in conjunction with one or more additional biomarkers does not depart from the concept, spirit and scope of the invention.

It is also well recognized in the art that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

It is also well recognized in the art that there are other Non-Alzheimer's neurological disorders related to those already mentioned that are hereby included within the scope of the invention including but not limited to Mild Cognitive Impairment, Cortical basal Ganglionic Degeneration, Amyotrophic Lateral Sclerosis, and any neurological disease or disorder, injury, depression or other psychiatric condition, or any other AD-Like disorder with symptoms similar to Alzheimer's disease that results from any other cause.

### Additional Tables

TABLE 3a

| Apolipoprotein E4 | | | | | | |
|---|---|---|---|---|---|---|
| Protein N5302 | n | Mean | ± | SE | % AMC | ANOVA-P |
| AMC | 75 | 81.4 | ± | 13.43 | 100% | |
| AD | 115 | 229.9 | ± | 18.74 | 282% | <0.0001 |
| PD | 12 | 0.0 | ± | — | 0% | |
| AD-Like + Mixed | 12 | 57.6 | ± | 23.20 | 71% | |

Table 3a: Mean level (ppm)±standard error (SE), percent change from the mean level of age-match normal control (AMC) and statistical differences from AMC (ANOVA-P) of blood serum Apolipoprotein E4 (spot N5302). Each sample (n) was run in triplicate on three separate 2D gels. These results are illustrated graphically in FIG. 3A.

TABLE 3b

| Apolipoprotein | ROC | | | | | |
|---|---|---|---|---|---|---|
| E4 Protein | N5302 [symbol] | | | | | |
| N5302 | cutoff | Sensitivity | Specificity | Area | SE | ROC-P |
| AMC | | | 74.7% | 0.66 | 0.020 | |
| AD | 0 | 55.1% | | | | <0.0001 |

Table 3b: Receiver Operator Characteristics (ROC) of blood serum Apolipoprotein E4 protein N5302 to distinguish between Alzheimer's disease patients and age-matched normal control (AMC) subjects, as reflected by sensitivity, specificity, and area under the curve, using biomarker N5302 cutoff concentration value of zero ppm. These results are illustrated graphically in FIG. 3B.

TABLE 4a

| Apolipoprotein | | | | | | |
|---|---|---|---|---|---|---|
| E4 Protein | | | | | | |
| N5302 | n | Mean | ± | SE | % N5302 > 0 | ANOVA-P |
| Age Matched | 75 | 81.4 | ± | 13.43 | 31% | |
| Controls | | | | | | |
| (AMC) | | | | | | |
| AD | 115 | 229.9 | ± | 18.74 | 58% | <0.0001 |
| Age Matched | 23 | 265.6 | ± | 34.91 | | |
| Controls | | | | | | |
| (AMC) | | | | | | |
| N5302 > 0 | | | | | | |
| AD N5302 > 0 | 67 | 394.7 | ± | 26.67 | | <0.0001 |

Table 4a: Mean level (ppm)±standard error (SE) and statistical differences from AMC (ANOVA-P) of blood serum Apolipoprotein E4 protein spot N5302 in all individuals and in individuals with detectable levels of Apolipoprotein E4 protein spot N5302 in the blood serum (N5302>0). The proportion of individuals with detectable levels of Apolipoprotein E4 protein spot N5302 (N5302>0) is presented as percentage of the total number of individuals in each category. Each sample (n) was run in triplicate on three separate 2D gels. These results are illustrated graphically in FIG. 4A.

TABLE 4b

| Apolipoprotein | | | | | | |
|---|---|---|---|---|---|---|
| E4 Protein | | | | | | |
| N5302 > | ROC | | | | | |
| 0:AD > | N5302 [symbol] | | | | | |
| AMC | cutoff | Sensitivity | Specificity | Area | SE | ROC-P |
| AMC | | | 50.7% | 0.61 | 0.040 | <0.0030 |
| AD | 159 | 64.2% | | | | |

Table 4b: Receiver Operator Characteristics (ROC) of the difference in blood serum concentrations of Apolipoprotein E4 protein N5302 to distinguish between Alzheimer's disease patients and age-matched normal control (AMC) subjects with detectable levels of Apolipoprotein E4 protein (N5302>0), as reflected by sensitivity, specificity, and area under the curve, using biomarker N5302 cutoff concentration value of 159 ppm. These results are illustrated graphically in FIG. 4B.

TABLE 5a

| Apolipoprotein | | | | | | |
|---|---|---|---|---|---|---|
| E3 Protein | | | | | | |
| N3314 | n | Mean | ± | SE | % AMC | ANOVA-P |
| Age Matched | 75 | 1047.7 | ± | 36.88 | 100% | |
| Controls (AMC) | | | | | | |
| Alzheimer's | 115 | 656.0 | ± | 27.01 | 63% | <0.0001 |
| Disease (AD) | | | | | | |
| Parkinson's | 12 | 230.8 | ± | 42.58 | 22% | |
| Disease (PD) | | | | | | |
| AD-Like + Mixed | 12 | 355.4 | ± | 48.89 | 34% | |

Table 5a: Mean level (ppm)±standard error (SE), percent change from the mean level of age-match normal controls (AMC) and statistical differences from AMC (ANOVA-P) of blood serum Apolipoprotein E3 protein spot N3314. Each sample (n) was run in triplicate on three separate 2D gels. These results are illustrated graphically in FIG. 5A.

TABLE 5b

| Apolipoprotein | ROC | | | | | |
|---|---|---|---|---|---|---|
| E3 Protein | N3314 [symbol] | | | | | |
| AD < AMC | cutoff | Sensitivity | Specificity | Area | SE | ROC-P |
| AMC | | | 64.0% | 0.71 | 0.022 | <0.0001 |
| AD | 804 | 64.1% | | | | |

Table 5b: Receiver Operator Characteristics (ROC) of the difference in blood serum concentrations of Apolipoprotein E3 protein spot N3314 to distinguish between Alzheimer's disease patients and age-matched normal control (AMC) subjects, as reflected by sensitivity, specificity, and area under the curve, using biomarker N3314 cutoff concentration value of AD<804 ppm. These results are illustrated graphically in FIG. 5B.

TABLE 6

| Apolipoprotein | | | | | | |
|---|---|---|---|---|---|---|
| E3 Protein | | | | | % AMC | |
| N3314 | n | Mean | ± | SE | N5302 = 0 | ANOVA-P |
| AMC N5302 = 0 | 52 | 1094.9 | ± | 44.10 | 100% | |
| AD N5302 = 0 | 48 | 872.9 | ± | 42.35 | 80% | P < 0.0004 |
| AMC N5302 > 0 | 23 | 940.9 | ± | 65.92 | 86% | |
| AD N5302 > 0 | 67 | 500.6 | ± | 30.75 | 46% | P < 0.0001 |

Table 6: Mean level (ppm)±standard error (SE), percent change from the mean level of age-match normal controls (AMC) and statistical differences from AMC (ANOVA-P) of blood serum Apolipoprotein E3 protein spot N3314 when Apolipoprotein E4 protein spot N5302 is detected (N5302>0) and not detected (N5302=0) in blood serum. Each sample (n) was run in triplicate on three separate 2D gels. These results are illustrated graphically in FIG. 6A.

TABLE 7
Apolipoprotein E3 protein N3314

| | AD vs. AMC | N3314 | n | Mean | SE |
|---|---|---|---|---|---|
| 7a. AD < AMC | ANOVA | AMC N5302 = 0 | 52 | 1084.8 | 44.10 |
| (N5302 = 0) | P < 0.0004 | AD N5302 = 0 | 48 | 872.9 | 42.35 |
| ANOVA | | | | | |
| | AD vs. AMC | N3314 | N3314 [symbol] | Sensitivity | Specificity |
| ROC | ROC | AMC N5302 = 0 | | | 53.8% |
| | | AD N5302 = 0 | 981 | 54.2% | |
| | | | Area | SE | ROC-P |
| | AD N5302 = 0 | [symbol] values | 0.50 | 0.030 | P < 0.0020 |
| | AD vs. AMC | N3314 | n | Mean | SE |
| 7b. AD < AMC | ANOVA | AMC N5302 = 0 | 23 | 940.9 | 55.92 |
| (N5302 > 0) | P < 0.0001 | AD N5302 = 0 | 87 | 502.0 | [symbol] .75 |
| ANOVA | | | | | |
| | AD vs. AMC | N3314 | N3314 [symbol] | Sensitivity | Specificity |
| ROC | ROC | AMC N5302 = 0 | | | 88.1% |
| | | AD N5302 > 0 | 807 | 58.2% | |
| | | | Area | SE | ROC-P |
| | AD N5302 > 0 | [symbol] values | 0.76 | 0.093 | <0.0001 |
| | AD vs. AD | N3314 | n | Mean | SE |
| 7c. AD (N5302 = 0) > | ANOVA | AD N5302 = 0 | 48 | 877.9 | 42.35 |
| AD (N5302 > 0) | P < 0.0001 | AD N5302 > 0 | 87 | 533.0 | 33.75 |
| ANOVA | | | | | |
| | AD vs. AD | N3314 | N3314 [symbol] | Sensitivity | Specificity |
| ROC | ROC | AD N5302 = 0 | | | 31.5% |
| | | AD N5302 > 0 | 651 | 21.6% | |
| | | | Area | SE | ROC-P |
| | AD N5302 > 0 | [symbol] values | 0.78 | 0.080 | <0.0001 |

[symbol]

indicates data missing or illegible when filed

Table 7: Receiver Operator Characteristics (ROC) of the difference in blood serum concentrations of Apolipoprotein E3 protein N3314 to distinguish between Alzheimer's disease patients and age-matched normal control (AMC) subjects, as reflected by sensitivity, specificity, and area under the curve for the ROC. Values of the concentration of Apolipoprotein E3 protein N3314 are calculated as AD<AMC for (a) individuals when Apolipoprotein E4 protein N5302 is not detected in the blood serum (N5302=0), using biomarker N3314 cutoff concentration value of AD<981 ppm; (b) individuals when Apolipoprotein E4 protein N5302 is detected in the blood serum (N5302>0), using biomarker N3314 cutoff concentration value of AD<607 ppm; and (c) differentiation between two types of Alzheimer's disease patients with Apolipoprotein E4 protein N5302 detected (N5302>0) vs. not detected (N5302=0) in the blood serum, using biomarker N3314 cutoff concentration value of AD (N5302>0)<651 ppm. These results are illustrated graphically in FIG. 7.

TABLE 8a

| Transthyretin Protein | | | % of | |
|---|---|---|---|---|
| N3307 | n | Mean ± SE | AMC | ANOVA-P |
| Age Matched | 75 | 481.9 ± 30.24 | 100% | |
| Controls (AMC) | | | | |
| AD | 115 | 347.0 ± 25.74 | 72% | <0.0001 |
| PD | 12 | 186.0 ± 29.83 | 39% | |
| AD-Like + Mixed | 11 | 171.3 ± 20.76 | 36% | |

Table 8a: Mean level (ppm)±standard error (SE), percent change from the mean level of age-match normal controls (AMC) and statistical differences from AMC (ANOVA-P) of blood serum Transthyretin Dimer (spot N3307). Each sample (n) was run in triplicate on three separate 2D gels. These results are illustrated graphically in FIG. 8A.

TABLE 8b

| Transthyretin | ROC | | | | | |
|---|---|---|---|---|---|---|
| Protein | N3307 < | | | | | |
| N3307 | cutoff | Sensitivity | Specificity | Area | SE | ROC-P |
| AMC | | | 60.9% | 0.66 | 0.023 | <0.0001 |
| AD | 333 | 60.9% | | | | |

Table 8b: Receiver Operator Characteristics (ROC) of the difference in blood serum concentrations of Transthyretin Dimer protein spot N3307 to distinguish between Alzheimer's disease patients and age-matched normal controls (AMC) subjects, as reflected by sensitivity, specificity, and area under the curve, using biomarker N3307 cutoff concentration value of AD<333 ppm. These results are illustrated graphically in FIG. 8B.

TABLE 9
a: Transthyretin “Dimer” Protein N3307

| Transthyretin Protein | | | | |
|---|---|---|---|---|
| N3307 | n | Mean ± SE | % of AMC | ANOVA-P |
| AMC N5302 = 0 | 52 | 508.3 ± 40.87 | 100% | |
| AD N5302 = 0 | 48 | 366.7 ± 23.74 | 72% | <0.0040 |
| AMC N5302 > 0 | 23 | 422.3 ± 33.72 | 83% | |
| AD N5302 > 0 | 67 | 332.9 ± 40.80 | 65% | >0.21 |

b: Transthyretin “Dimer” Protein N3307

| Transthyretin Protein | ROC | | | | | |
|---|---|---|---|---|---|---|
| N3307 AD < AMC | N3307 < cutoff | Sensitivity | Specificity | Area | SE | ROC-P |
| AMC N5302 = 0 | | | 55.1% | 0.60 | 0.033 | |
| AD N5302 = 0 | 352 | 54.9% | | | | <0.0020 |
| AMC N5302 > 0 | | | 65.2% | 0.73 | 0.030 | |
| AD N5302 > 0 | 308 | 65.2% | | | | <0.0001 |

Table 9: (a) Mean level (ppm)±standard error (SE), percent change from the mean level of age-match normal controls (AMC) and statistical differences from AMC (ANOVA-P) of blood serum Transthyretin Dimer (spot N3307), when N5302 is detected (N5302>0) and not detected (N5302=0) in the blood serum. (b) Receiver Operator Characteristics (ROC) of the difference in blood serum concentrations of Transthyretin Dimer protein N3307 to distinguish between Alzheimer's disease patients and age-matched normal control (AMC) subjects, as reflected by sensitivity, specificity, and area under the curve for the ROC. Values are calculated for individuals when Apolipoprotein E4 protein N5302 is detected (N5302>0) and not detected (N5302=0) in the blood serum, using biomarker N3307 cutoff values of AD<308 and AD<352 ppm, respectively. These results are illustrated graphically in FIGS. 9A and B, respectively.

TABLE 10a

| Complement Factor | | | % | |
|---|---|---|---|---|
| H/HS Protein N4411 | n | Mean ± SE | AMC | ANOVA-P |
| Age Matched Controls | 75 | 296.8 ± 20.85 | 100% | |
| (AMC) | | | | |
| Alzheimer's Disease (AD) | 115 | 374.5 ± 17.66 | 126% | <0.0040 |
| Parkinson's Disease (PD) | 12 | 435.9 ± 59.48 | 147% | |
| AD-Like + Mixed | 12 | 258.8 ± 36.75 | 87% | |

Table 10a: Mean level (ppm)±standard error (SE), percent change from the mean level of age-match normal controls (AMC) and statistical differences from AMC (ANOVA-P) of blood serum Complement Factor H/Hs protein (spot N4411). Each sample (n) was run in triplicate on three separate 2D gels. These results are illustrated graphically in FIG. 10A.

TABLE 10b

| Complement | | | | | | |
|---|---|---|---|---|---|---|
| Factor H/HS | | | | | | |
| Protein | ROC | | | | | |
| N4411 | N4411 > | | | | | |
| AD > AMC | cutoff | Sensitivity | Specificity | Area | SE | ROC-P |
| AMC | | | 57.3% | 0.59 | 0.024 | |
| AD | >261 | 57.4% | | | | <0.0002 |

Table 10b: Summary statistics for the graph in FIG. 10b; Receiver Operator Characteristics (ROC) of the differences in concentration in blood serum of Complement Factor H/Hs protein spot N4411, where AD>AMC (AUC=0.59±0.024) to distinguish between Alzheimer's disease patients and age-matched normal control (AMC) subjects, as reflected by sensitivity, specificity, and area under the curve, using biomarker N4411 cutoff concentration value of AD>261 ppm. These results are illustrated graphically in FIG. 10B.

TABLE 11
a:

| Complement Factor H | | | | |
|---|---|---|---|---|
| Protein N4411 | n | Mean ± SE | % of AMC | ANOVA-P |
| AMC N5302 = 0 | 52 | 287.3 ± 24.65 | 100% | |
| AD N5302 = 0 | 48 | 438.2 ± 29.04 | 153% | <0.0001 |
| AMC N5302 > 0 | 23 | 319.3 ± 39.10 | 111% | |
| AD N5302 > 0 | 67 | 328.9 ± 21.54 | 114% | >0.80 |

b:

| Complement Factor H Protein | ROC | | | | | |
|---|---|---|---|---|---|---|
| N4411 AD > ASMC | N4411 > cutoff | Sensitivity | Specificity | Area | SE | ROC-P |
| AMC N5302 = 0 | | | 62.2% | 0.64 | 0.032 | |
| AD N5302 = 0 | 270 | 62.5% | | | | <0.0001 |
| AMC N5302 > 0 | | | 49.3% | 0.53 | 0.041 | |
| AD N5302 > 0 | 273 | 49.3% | | | | P > 0.22 |

Table 11: (a) Mean level (ppm)±standard error (SE), percent change from the mean level of age-match normal controls (AMC) and statistical differences from AMC (ANOVA-P) of blood serum Complement Factor H/Hs protein spot N4411, when N5302 is detected (N5302>0) and not detected (N5302=0) in the blood serum. (b) Receiver Operator Characteristics (ROC) of the difference in blood serum concentrations of Complement Factor H/Hs protein spot N4411 to distinguish between Alzheimer's disease patients and age-matched normal control (AMC) subjects, as reflected by sensitivity, specificity, and area under the curve for the ROC. Values are calculated for individuals when Apolipoprotein E4 protein N5302 is detected (N5302>0) and not detected (N5302=0) in the blood serum, using biomarker N4411 cutoff concentration values of AD>273 and AD>270 ppm, respectively. These results are illustrated graphically in FIGS. 11A and B, respectively.

TABLE 12a

| Complement Factor | | | % AMC | |
|---|---|---|---|---|
| Bb Protein N7616 | n | Mean ± SE | N5302 = 0 | ANOVA-P |
| AMC | 75 | 276.4 ± 16.59 | 100% | |
| AD | 115 | 298.7 ± 11.41 | 108% | P > 0.110 |
| PD | 12 | 368.2 ± 22.34 | 133% | |
| AD-Like + Mixed | 12 | 311.0 ± 25.81 | 113% | |

Table 12a: Mean level (ppm)±standard error (SE), percent change from the mean level of age-match normal controls (AMC) and statistical differences from AMC (ANOVA-P) of blood serum Complement Factor Bb protein (spot N7616). Each sample (n) was run in triplicate on three separate 2D gels. These results are illustrated graphically in FIG. 12A.

TABLE 12b

| Complement | | | | | | |
|---|---|---|---|---|---|---|
| Factor Bb | | | | | | |
| Protein | ROC | | | | | |
| N7616 | N5302 < | | | | | |
| AD > AMC | cutoff | Sensitivity | Specificity | Area | SE | ROC-P |
| AMC | | | 52.4% | 0.53 | 0.024 | |
| AD | 233 | 52.5% | | | | >0.09 |

Table 12b: Summary statistics for the graph in FIG. 12b; Receiver Operator Characteristics (ROC) of the differences in concentration in blood serum of Complement Factor Bb protein (spot N7616) where AD>AMC (AUC=0.53±0.024), to distinguish between Alzheimer's disease patients and age-matched normal control (AMC) subjects, as reflected by sensitivity, specificity, and area under the curve, where AD>AMC using biomarker N7616 cutoff concentration value of AD>233 ppm. These results are illustrated graphically in FIG. 12B.

TABLE 13
a

| Complement Facto | | | | |
|---|---|---|---|---|
| Bb Protein N7616 | n | Mean ± SE | % of AMC | ANOVA-P |
| AMC N5302 = 0 | 52 | 262.2 ± 12.88 | 100% | |
| AD N5302 = 0 | 48 | 347.9 ± 21.14 | 133% | <0.0006 |
| AMC N5302 > 0 | 23 | 308.4 ± 45.60 | 118% | |
| AD N5302 > 0 | 67 | 263.4 ± 11.85 | 100% | >0.17 |

b

| Complement Facto Bb | ROC | | | | | |
|---|---|---|---|---|---|---|
| Protein N7616 AD > 0 | N7616 > cutoff | Sensitivity | Specificity | Area | SE | ROC-P |
| AMC N5302 = 0 | | | 55.8% | 0.59 | 0.033 | |
| AD N5302 = 0 | 237 | 55.6% | | | | <0.0040 |
| AMC N5302 > 0 | | | 49.3% | 0.48 | 0.039 | |
| AD N5302 > 0 | 229 | 50.7% | | | | P > 0.6 |

Table 13: (a) Mean level (ppm)±standard error (SE), percent change from the mean level of age-match normal control (AMC) and statistical differences from AMC (ANOVA-P) of blood serum Complement Factor Bb protein biomarker spot N7616, when Apolipoprotein E4 protein N5302 is detected (N5302>0) and not detected (N5302=0) in the blood serum. (b) Receiver Operator Characteristics (ROC) of the difference in blood serum concentrations of Complement Factor Bb protein biomarker spot N7616 to distinguish between Alzheimer's disease patients and age-matched normal control (AMC) subjects, as reflected by sensitivity, specificity, and area under the curve for the ROC. Values are calculated for individuals when N5302 is detected (N5302>0) and not detected (N5302=0) in the blood serum, where AD>AMC using biomarker N7616 cutoff concentration values of AD>229 and AD>237 ppm, respectively. These results are illustrated graphically in FIGS. 13A and B, respectively.

TABLE 14

| | Complement C3c1 Phosphoprotein N7310 | | | | | |
|---|---|---|---|---|---|---|
| | Acquired Auto-Immune Inflammation | | | | | |
| | | | | | | Mean % |
| | N7310 | n | Mean | Median | SE | AMC |
| 14a: | Age Matched | 75 | 315.8 | 241.7 | 10.57 | 100% |
| | Control | | | | | |
| | AD | 115 | 884.5 | 330.0 | 80.81 | 280% |
| | PD | 12 | 1380.7 | 1230.0 | 190.20 | 443% |
| | AD-Like and Mixed | 12 | 300.2 | 213.3 | 72.23 | 07% |
| | Complement C3dg Protein N1511 | | | | | |
| | Innate + Acquired Auto-Immune Inflammation | | | | | |
| | | | | | | Mean % |
| | N1511 | n | Mean | Median | SE | AMC |
| 14b: | Age Matched | 75 | 400.5 | 74.1 | 8.65 | 100% |
| | Control | | | | | |
| | AD | 115 | 457.9 | 190.1 | 31.51 | 416% |
| | PD | 12 | 792.3 | 642.7 | 75.30 | 723% |
| | AD-Like and Mixed | 12 | 400.7 | 250.2 | 50.23 | 371% |
| | Complement C3c2a Protein N9311 | | | | | |
| | Innate Immune Inflammation | | | | | |
| | | | | | | Mean % |
| | N9311 | n | Mean | Median | SE | AMC |
| 14c: | Age Matched | 75 | 305.7 | 250.1 | 12.03 | 100% |
| | Control | | | | | |
| | AD | 115 | 580.5 | 258.8 | 35.07 | 100% |
| | PD | 12 | 728.1 | 635.3 | 5.37 | 238% |
| | AD-Like and Mixed | 12 | 802.1 | 437.8 | 92.01 | 197% |
| | Complement C3Sum = N7310 + N9311 + N1511 | | | | | |
| | Innate + Acquired Auto-Immune Inflammation | | | | | |
| | | | | | | Mean % |
| | C3Sum | n | Mean | Median | SE | AMC |
| 14d: | Age Matched | 75 | 731.1 | 843.4 | 29.19 | 100% |
| | Control | | | | | |
| | AD | 115 | 1922.0 | 929.6 | 112.72 | 283% |
| | PD | 12 | 2920.2 | 3220.0 | 259.80 | 399% |
| | AD-Like and Mixed | 12 | 1314.0 | 1155.0 | 133.23 | 180% |

Table 14: Mean level (ppm)±standard error (SE), percent change from the mean level of age-match normal controls (AMC) and Median (50^{th} percentile) of blood serum a) Complement C3c1 phosphoprotein (spot N7310); b) Complement C3dg protein spot N1511; c) Complement C3c2a protein (spot N9311); Complement C3Sum (N7310+N1511+N9311). Each sample (n) was run in triplicate on three separate 2D gels. These results are illustrated graphically in FIG. 14.

TABLE 15

| | N1511 | | | | | |
|---|---|---|---|---|---|---|
| | ROC | N1511 > cutoff | Sensitivity | Specificity | ROC-P | ANOVA-P |
| 15a | AD > AMC | >105 | 60.9% | 60.9% | <0.0001 | <0.0001 |
| | N7310 | | | | | |
| | ROC | N7310 > cutoff | Sensitivity | Specificity | ROC-P | ANOVA-P |
| 15b | AD > AMC | >273 | 65.9% | 55.5% | <0.0001 | <0.0001 |
| | N9311 | | | | | |
| | ROC | N9311 > cutoff | Sensitivity | Specificity | ROC-P | ANOVA-P |
| 15c | AD > AMC | >273 | 53.9% | 53.8% | <0.0001 | <0.0001 |
| | C3Sum | | | | | |
| | ROC | C3Sum > cutoff | Sensitivity | Specificity | ROC-P | ANOVA-P |
| 15d | AD > AMC | >710 | 55.1% | 55.1% | <0.0001 | <0.0001 |

Table 15: Receiver Operator Characteristics (ROC) of the differences in blood serum concentrations of a) Complement C3dg protein (spot N1511); b) Complement C3c1 phosphoprotein (spot N7310); Complement C3c2a protein (spot N9311); and C3Sum (N1511+N7310+N9311) to distinguish between Alzheimer's disease patients and age-matched normal control (AMC) subjects, as reflected by sensitivity, specificity, and statistical significance, where AD>AMC using cutoff concentration values for each at Receiver Operator Characteristics (ROC) of the difference in blood serum concentrations of AD>105, AD>273, AD>272, and AD>710 ppm, respectively. These results are illustrated graphically in FIG. 15.

TABLE 16
16a

| | Complement C3c1 Phosphoprotein N7310 | | | | | | Complement C3c2a Protein N9311 | | | | | |
|---|---|---|---|---|---|---|---|---|---|---|---|---|
| Summary | Acquired AutoImmune Inflammation | | | | | | Innate Inflammation | | | | | |
| Statistics | N7310 | n | Mean | Median | SE | % Change | N9311 | n | Mean | Median | SE | % Change |
| AMC [symbol] = [symbol] | ANOVA | 62 | 332.6 | 270.3 | 2100 | 303% | ANOVA | 62 | 335.1 | 278.2 | 16.11 | 128% |
| AD [symbol] = [symbol] | P < 0.0001 | 18 | [symbol] | [symbol] | 110.16 | | P < 0.0001 | 18 | [symbol] | 298.1 | 50.15 | |
| AMC [symbol] 0 | ANOVA | 23 | [symbol] | 180.2 | 403.7 | [symbol] % | ANOVA | 23 | 251.9 | 225.1 | 10.34 | 206% |
| AD [symbol] 0 | P < 0.0001 | 67 | 743.1 | 296.3 | 666.7 | | P < 0.0001 | 61 | 540.0 | 296.7 | 41.43 | |
| AD [symbol] = 0 | ANOVA | 18 | [symbol] | [symbol] | 110.16 | 70% | ANOVA | 18 | 53.1 | 298.1 | 53.15 | 85% |
| AD [symbol] 0 | P < 0.0001 | 67 | 745.1 | 296.3 | 65.57 | | P > 0.11 | 67 | 540.0 | [symbol] | 41.43 | |

16b

| | Complement C3dg Protein N1511 | | | | | | Complement C3Sum = N1511 + N7310 + N9311 | | | | | |
|---|---|---|---|---|---|---|---|---|---|---|---|---|
| Summary | Innate + Acquired AutoImmune Inflammation | | | | | | Innate + Acquired AutoImmune Inflammation | | | | | |
| Statistics | N1511 | n | Mean | Median | SE | % Change | C3Sum | n | Mean | Median | SE | % Change |
| AMC N5302 = 0 | ANOVA | 62 | 105.8 | 69.4 | 103.0 | 511% | ANOVA | 62 | 753.4 | 604.8 | 30.59 | 295% |
| AD N5302 = 0 | P < 0.0001 | 18 | 510.3 | 210.3 | 95.53 | | P < 0.0001 | 18 | 2253.3 | [symbol] | 203.07 | |
| AMC N5302 > 0 | ANOVA | 23 | 110.0 | 199 | 1590 | 300% | ANOVA | 23 | [symbol] | [symbol] | 56.11 | 266% |
| AD N5302 > 0 | P < 0.0001 | 67 | [symbol] | 167.6 | 36.82 | | P < 0.0001 | 61 | [symbol] | 116.0 | 136.30 | |
| AD N5302 = 0 | ANOVA | 18 | 540.3 | [symbol] | 55.98 | 74% | ANOVA | 18 | [symbol] | [symbol] | 203.07 | 78% |
| AD N5302 > 0 | P < 0.0001 | 67 | [symbol] | 167.6 | 36.82 | | P < 0.0001 | 67 | 1687.0 | 715.0 | 136.38 | |

[symbol]

indicates data missing or illegible when filed

Table 16: Mean level (ppm)±standard error (SE), Median (50^{th} percentile) and percent change in blood serum levels of a) Complement C3c1 phosphoprotein (spot N7310), Complement C3c2a protein (spot N9311), and b) Complement C3dg protein spot N1511, and Complement C3Sum (N7310+N1511+N9311) when Apolipoprotein E4 protein N5302 is not detected (N5302=0) and detected (N5302>0) in the blood serum. These results are illustrated graphically in FIG. 16.

TABLE 17
17a

| | | Complement C3c1 | | | | Complement C3c2a | | | | |
|---|---|---|---|---|---|---|---|---|---|---|
| | | Phosphoprotein N7310 | | | | Protein N9311 | | | | |
| | | Acquired Auto-Immune Inflammation | | | | Innate Inflammation | | | | |
| Receiver Operator | | N7310 | | | | N9311 | | | | |
| Characteristics | | cutoff | Sensitivity | Specificity | ROC-P | cutoff | Sensitivity | Specificity | ROC-P | Class |
| AD vs. AMC | AMC [symbol] | | | [symbol] | [symbol] | | | [symbol] | [symbol] | Not AD |
| at Sensitivity [symbol] | [symbol] | [symbol] | [symbol] | | | [symbol] | [symbol] | | | AD |
| Specificity | | | | | | | | | | |
| AD vs. AMC | AMC [symbol] | | | [symbol] | [symbol] | | | [symbol] | [symbol] | Not AD |
| at Sensitivity | AD [symbol] | [symbol] | [symbol] | | | [symbol] | [symbol] | | | AD |
| Specificity | | | | | | | | | | |
| AD vs. AD | AD [symbol] | [symbol] | [symbol] | | [symbol] | [symbol] | [symbol] | | [symbol] | AD |
| at Sensitivity | AD [symbol] | | | [symbol] | | | | [symbol] | | AD |
| Specificity | | | | | | | | | | |

17b

| | | | | | | Complement C3Sum = N1511 + | | | | |
|---|---|---|---|---|---|---|---|---|---|---|
| | | Complement C3dg Protein N1511 | | | | N7310 + N9311 | | | | |
| | | Innate + Acquired Immune Inflammation | | | | Innate + Acquired Immune Inflammation | | | | |
| Receiver Operator | | N1511 | | | | C3Sum | | | | |
| Characteristics (ROC) | | cutoff | Sensitivity | Specificity | ROC-P | cutoff | Sensitivity | Specificity | ROC-P | Class |
| AD vs. AMC | AMC N5302 = 0 | | | 62.8% | <0.0001 | | | 56.4% | <0.0001 | Not AD |
| at Sensitivity [symbol] | AD N5302 = 0 | >107 | 63.2% | | | >743 | 56.3% | | | AD |
| Specifivity | | | | | | | | | | |
| AD vs. AMC | AMC N5302 > 0 | | | 58.0% | <0.0001 | | | 58.0% | <0.0001 | Not AD |
| at Sensitivity [symbol] | AD N5302 > 0 | >106 | 58.2% | | | >635 | 58.2% | | | AD |
| Specifivity | | | | | | | | | | |
| AD vs. AD | AD N5302 = 0 | >196 | 51.4% | | >0.110 | >769 | 56.3% | | <0.0009 | AD |
| at Sensitivity [symbol] | AD N5302 > 0 | | | 51.2% | | | | 56.3% | | Not AD |
| Specifivity | | | | | | | | | | |

[symbol]

indicates data missing or illegible when filed

Table 17: Receiver Operator Characteristics (ROC) of the difference in blood serum concentrations of a) Complement C3c1 phosphoprotein (spot N7310), Complement C3c2a protein (spot N9311), and b) Complement C3dg protein (spot N1511), and C3Sum (N1511+N7310+N9311) to distinguish between Alzheimer's disease patients and age-matched normal control (AMC) subjects and between two Alzheimer's disease patients, when Apolipoprotein E4 protein spot N5302 was not detected (N5302=0) and detected (N5302>0), as reflected by sensitivity, specificity, and statistical significance, using a characteristic cutoff concentration value for each biomarker. These results are illustrated graphically in FIG. 17.

TABLE 18a

| Haptoglobin HP-1 Total Proteins | | | | |
|---|---|---|---|---|
| N1514 + N2401 + N2407 + N3409 | n | Mean ± SE | % AMC | ANOVA-P |
| AMC | 75 | 29434.0 ± 703.64 | 100% | |
| AD | 115 | 32898.4 ± 618.74 | 112% | 0.0028 |
| PD | 12 | 29387.4 ± 1574.31 | 100% | |
| AD-Like + Mixed | 12 | 32316.8 ± 2522.66 | 110% | |

Table 18a: Mean level (ppm)±standard error (SE), percent change from the mean level of age-match normal control (AMC) and statistical significance of blood serum Total Haptoglobin Hp-1 proteins (Sum of spots N1514+N2401+N2407+N3409). Each sample (n) was run in triplicate on three separate 2D gels. These results are illustrated graphically in FIG. 22A.

TABLE 18b

| | ROC | | | | | |
|---|---|---|---|---|---|---|
| Haptoglobin HP-1 Total Proteins | Hp-1 Total > | | | | | |
| N1514 + N2401 + N2407 + N3409 | cutoff | Sensitivity | Specificity | Area | SE | ROC-P |
| AMC | | | 56.0% | 0.59 | 0.024 | |
| AD | 30136 | 56.2% | | | | <0.0001 |

Table 18b: Receiver Operator Characteristics (ROC) of the difference in blood serum concentrations of

Total Haptoglobin Hp-1 proteins (Sum of spots N1514+N2401+N2407+N3409) to distinguish between Alzheimer's disease patients and age-matched normal control (AMC) subjects, as reflected by sensitivity, specificity, area under the curve of ROC and statistical significance, where AD>AMC using cutoff value at AD>30136 ppm. These results are illustrated graphically in FIG. 22B.

TABLE 19a

| Total HP-1 | ROC | | | | | |
|---|---|---|---|---|---|---|
| Proteins N1514 + | Total | | | | | |
| N2401 + N2407 + | HP-1 < | Sensi- | Speci- | | | |
| N3409 AD > AMC | cutoff | tivity | ficity | Area | SE | ROC-P |
| AMC N5302 = 0 | | | 64.7% | 0.68 | 0.031 | |
| AD N5302 = 0 | 30216 | 64.6% | | | | <0.0001 |
| AMC N5302 > 0 | | | 44.9% | 0.47 | 0.040 | |
| AD N5302 > 0 | 31768 | 44.8% | | | | >0.14 |

Table 19a: Mean level (ppm)±standard error (SE), percent change from the mean level of age-match normal control (AMC) and statistical differences from AMC (ANOVA-P) of blood serum total Haptoglobin Hp-1 proteins (Sum of spots N1514+N2401+N2407+N3409), when Apolipoprotein E4 protein N5302 is detected (N5302>0) and not detected (N5302=0) in the blood serum. These results are illustrated graphically in FIG. 25A.

TABLE 19b

| Total HP-1 | ROC | | | | | |
|---|---|---|---|---|---|---|
| Proteins N1514 + | Total | | | | | |
| N2401 + N2407 + | HP-1 < | Sensi- | Specifi- | | | |
| N3409 AD > AMC | cutoff | tivity | city | Area | SE | ROC-P |
| AMC N5302 = 0 | | | 64.7% | 0.68 | 0.031 | |
| AD N5302 = 0 | 30216 | 64.6% | | | | <0.0001 |
| AMC N5302 > 0 | | | 44.9% | 0.47 | 0.040 | |
| AD N5302 > 0 | 31768 | 44.8% | | | | >0.14 |

Table 19b: Receiver Operator Characteristics (ROC) of the difference in blood serum concentrations of total Haptoglobin Hp-1 proteins (Sum of spots N1514+N2401+N2407+N3409) to distinguish between Alzheimer's disease patients and age-matched normal control (AMC) subjects, as reflected by sensitivity, specificity, and area under the curve for the ROC. Values are calculated for each group when Apolipoprotein E4 protein N5302 is detected (N5302>0) and not detected (N5302=0) in the blood serum, where AD>AMC using cutoff values of AD>31768 and AD>30216 ppm, respectively. These results are illustrated graphically in FIG. 25B.

TABLE 20a

| Inter-alpha-trypsin | | | | |
|---|---|---|---|---|
| Inhibitor Heavy Chain | | | | |
| (H4) Related 35 KD | | | | |
| Protein N2307 | n | Mean ± SE | % AMC | ANOVA-P |
| AMC | 75 | 241.0 ± 13.74 | 100% | |
| AD | 115 | 410.2 ± 20.41 | 170% | <0.0001 |
| PD | 12 | 408.6 ± 54.66 | 170% | |
| AD-Like + Mixed | 12 | 327.6 ± 51.07 | 136% | |

Table 20a: Mean level (ppm)±standard error (SE), percent change from the mean level of age-match normal control (AMC) and statistical significance of blood serum Inter-alpha-trypsin inhibitor heavy chain (H4) related 35 KD protein (spot N2307). Each sample (n) was run in triplicate on three separate 2D gels. These results are illustrated graphically in FIG. 26A.

TABLE 20b

| Inter-alpha- | | | | | | |
|---|---|---|---|---|---|---|
| trypsin Inhibitor | | | | | | |
| Heavy | | | | | | |
| Chain (H4) | | | | | | |
| Related 35 KD | ROC | | | | | |
| Protein N2307 | N2307 > | Sensi- | Speci- | | | |
| AD > AMC | cutoff | tivity | ficity | Area | SE | ROC-P |
| AMC | | | 58.2% | 0.62 | 0.023 | |
| AD | 210 | 58.0% | | | | <0.0001 |

Table 20b: Receiver Operator Characteristics (ROC) of the difference in blood serum concentrations of Inter-alpha-trypsin inhibitor heavy chain (H4) related 35 KD protein (spot N2307) to distinguish between Alzheimer's disease patients and age-matched normal control (AMC) subjects, as reflected by sensitivity, specificity, area under the curve of ROC and statistical significance, where AD>AMC using cutoff concentration value at AD>210 ppm. These results are illustrated graphically in FIG. 26B.

TABLE 21a

| Inter-alpha-trypsin | | | | |
|---|---|---|---|---|
| inhibitor heavy chain | | | | |
| (H4) Related 35 KD | | | % AMC | |
| Protein N2307 | n | Mean ± SE | N5302 = 0 | ANOVA |
| AMC N5302 = 0 | 52 | 215.9 ± 14.49 | 100% | |
| AD N5302 = 0 | 48 | 457.6 ± 36.34 | 212% | P < 0.0001 |
| AMC N5302 > 0 | 23 | 297.9 ± 29.62 | 138% | |
| AD N5302 > 0 | 67 | 376.1 ± 23.23 | 174% | P > 0.06 |

Table 21a: Mean level (ppm)±standard error (SE), percent change from the mean level of age-match normal control (AMC) and statistical differences from AMC (ANOVA-P) of blood serum Inter-alpha-trypsin inhibitor heavy chain (H4) related 35 KD protein (spot N2307), when Apolipoprotein E4 protein N5302 is detected (N5302>0) and not detected (N5302=0) in the blood serum. These results are illustrated graphically in FIG. 27A.

TABLE 21b

| Inter-alpha- | | | | | | |
|---|---|---|---|---|---|---|
| trypsin inhibitor | | | | | | |
| heavy chain (H4) | | | | | | |
| Related 35 KD | ROC | | | | | |
| Protein N2307 | N2307 < | Sensi- | Speci- | | | |
| AD > AMC | cutoff | tivity | ficity | Area | SE | ROC-P |
| AMC N5302 = 0 | | | 61.5% | 0.68 | 0.031 | |
| AD N5302 = 0 | 211 | 61.8% | | | | <0.0001 |
| AMC N5302 > 0 | | | 50.7% | 0.54 | 0.038 | |
| AD N5302 > 0 | 224 | 50.7% | | | | >0.14 |

Table 21b: Receiver Operator Characteristics (ROC) of the difference in blood serum concentrations of Inter-alpha-trypsin inhibitor heavy chain (H4) related 35 KD protein spot N2307 to distinguish between Alzheimer's disease patients and age-matched normal control (AMC) subjects, as reflected by sensitivity, specificity, and area under the curve for the ROC. Values are calculated for each group when Apolipoprotein E4 protein N5302 is detected (N5302>0) and not detected (N5302=0) in the blood serum, where AD>AMC using cutoff concentration values of AD>224 and AD>211 ppm, respectively. These results are illustrated graphically in FIG. 27B.

TABLE 22a

| Immunoglobulin | | | | |
|---|---|---|---|---|
| Light Chain Protein | | | | |
| N6224 | n | Mean ± SE | % AMC | ANOVA-P |
| AMC | 75 | 461.1 ± 16.74 | 100% | |
| AD | 115 | 369.4 ± 15.35 | 80% | <0.0002 |
| PD | 12 | 336.1 ± 24.24 | 73% | |
| AD-Like + Mixed | 12 | 329.1 ± 39.04 | 71% | |

Table 22a: Mean level (ppm)±standard error (SE), percent change from the mean level of age-match normal control (AMC) and statistical significance of blood serum Immunoglobulin light chain protein (spot N6224). Each sample (n) was run in triplicate on three separate 2D gels. These results are illustrated graphically in FIG. 29A.

TABLE 22b

| Immunoglobulin | | | | | | |
|---|---|---|---|---|---|---|
| Light Chain | ROC | | | | | |
| Protein N6224 | N6224 < | Sensi- | Speci- | | | |
| AD < AMC | cutoff | tivity | ficity | Area | SE | ROC-P |
| AMC | | | 59.6% | 0.64 | 0.023 | |
| AD | 368 | 59.7% | | | | <0.0001 |

Table 22b: Receiver Operator Characteristics (ROC) of the difference in blood serum concentrations of Immunoglobulin light chain protein (spot N6224) to distinguish between Alzheimer's disease patients and age-matched normal control (AMC) subjects, as reflected by sensitivity, specificity, area under the curve of ROC and statistical significance, where AD<AMC using cutoff concentration value at AD<368 ppm. These results are illustrated graphically in FIG. 29B.

TABLE 23a

| Immunoglobulin | | | | |
|---|---|---|---|---|
| Light Chain Protein | | | % AMC | |
| N6224 | n | Mean ± SE | N5302 = 0 | ANOVA |
| AMC N5302 = 0 | 52 | 452.4 ± 19.81 | 100% | |
| AD N5302 = 0 | 48 | 391.2 ± 30.15 | 86% | P > 0.08 |
| AMC N5302 > 0 | 23 | 480.7 ± 31.27 | 106% | |
| AD N5302 > 0 | 67 | 353.7 ± 15.06 | 78% | P < 0.0007 |

Table 23a: Mean level (ppm)±standard error (SE), percent change from the mean level of age-match normal control (AMC) and statistical differences from AMC (ANOVA-P) of blood serum Immunoglobulin light chain protein (spot N6224), when Apolipoprotein E4 protein N5302 is detected (N5302>0) and not detected (N5302=0) in the blood serum. These results are illustrated graphically in FIG. 30B.

TABLE 23b

| Immunoglobulin | | | | | | |
|---|---|---|---|---|---|---|
| Light Chain | ROC | | | | | |
| Protein N6224 | N6221 < | Sensi- | Speci- | | | |
| AD < AMC | cutoff | tivity | ficity | Area | SE | ROC-P |
| AMC N5302 = 0 | | | 57.1% | 0.62 | 0.032 | |
| AD N5302 = 0 | 368 | 56.9% | | | | <0.0003 |
| AMC N5302 > 0 | | | 62.3% | 0.68 | 0.036 | |
| AD N5302 > 0 | 378 | 62.7% | | | | <0.0001 |

Table 23b: Receiver Operator Characteristics (ROC) of the difference in blood serum concentrations of Immunoglobulin light chain protein (spot N6224) to distinguish between Alzheimer's disease patients and age-matched normal control (AMC) subjects, as reflected by sensitivity, specificity, and area under the curve for the ROC. Values are calculated for each group when N5302 is detected (N5302>0) and not detected (N5302=0) in the blood serum, where AD<AMC using cutoff values of AD<378 and AD<368 ppm, respectively. These results are illustrated graphically in FIG. 30B.

TABLE 24a

| Apolipoprotein | | | | |
|---|---|---|---|---|
| A-IV Protein | | | | |
| N2502 | n | Mean ± SE | % AMC | ANOVA-P |
| AMC | 75 | 2915.6 ± 115.15 | 100% | |
| AD | 115 | 2341.8 ± 74.61 | 80% | <0.0001 |
| PD | 12 | 1737.8 ± 180.58 | 60% | |
| AD-Like + Mixed | 12 | 1934.2 ± 191.21 | 66% | |

Table 24a: Mean level (ppm)±standard error (SE), percent change from the mean level of age-match normal controls (AMC) and statistical significance of blood serum Apolipoprotein A-IV protein (spot N2502). Each sample (n) was run in triplicate on three separate 2D gels. These results are illustrated graphically in FIG. 31B.

TABLE 24b

| Apolipoprotein | ROC | | | | | |
|---|---|---|---|---|---|---|
| A-IV Protein | N2502 < | Sensi- | Speci- | | | |
| N2502 | cutoff | tivity | ficity | Area | SE | ROC-P |
| AMC | | | 59.1% | 0.64 | 0.023 | |
| AD | 2465 | 59.1% | | | | <0.0001 |

Table 24b: Receiver Operator Characteristics (ROC) of the difference in blood serum concentrations of Apolipoprotein A-IV protein (spot N2502) to distinguish between Alzheimer's disease patients and age-matched normal control (AMC) subjects, as reflected by sensitivity, specificity, area under the curve of ROC and statistical significance, where AD<AMC using N2502 cutoff value at AD<2465 ppm. These results are illustrated graphically in FIG. 31B.

TABLE 25a

| Apolipoprotein | | | | |
|---|---|---|---|---|
| A-IV Protein | | | % AMC | |
| N2502 | n | Mean ± SE | N5302 = 0 | ANOVA |
| AMC N5302 = 0 | 52 | 2993.5 ± 155.71 | 100% | |
| AD N5302 = 0 | 48 | 2339.1 ± 131.43 | 78% | P < 0.0003 |
| AMC N5302 > 0 | 23 | 2739.7 ± 129.70 | 92% | |
| AD N5302 > 0 | 67 | 2343.8 ± 87.10 | 78% | P < 0.03 |

Table 25a: Mean level (ppm)±standard error (SE), percent change from the mean level of age-match normal control (AMC) and statistical differences from AMC (ANOVA-P) of blood serum Apolipoprotein A-IV protein (spot N2502), when Apolipoprotein E4 protein N5302 is detected (N5302>0) and not detected (N5302=0) in the blood serum. These results are illustrated graphically in FIG. 32A.

TABLE 25b

| Apolipoprotein | | | | | | |
|---|---|---|---|---|---|---|
| A-IV Protein | ROC | | | | | |
| N2502 | N2502 < | Sensi- | Speci- | | | |
| AD < AMC | cutoff | tivity | ficity | Area | SE | ROC-P |
| AMC N5302 = 0 | | | 58.3% | 0.65 | 0.032 | |
| AD N5302 = 0 | 2412 | 58.3% | | | | <0.0001 |
| AMC N5302 > 0 | | | 62.3% | 0.64 | 0.039 | |
| AD N5302 > 0 | 2588 | 62.2% | | | | <0.0003 |

Table 25b: Receiver Operator Characteristics (ROC) of the difference in blood serum concentrations of

Apolipoprotein A-IV protein (spot N2502) to distinguish between Alzheimer's disease patients and age-matched normal control (AMC) subjects, as reflected by sensitivity, specificity, and area under the curve for the ROC. Values are calculated for each group when Apolipoprotein E4 protein N5302 is detected (N5302>0) and not detected (N5302=0) in the blood serum, where AD<AMC using cutoff concentration values of AD<2588 and AD2412 ppm, respectively. These results are illustrated graphically in FIG. 32B.

TABLE 26

| Linear Discriminant | | | |
|---|---|---|---|
| Function | Biomarkers Employed in Discriminant Function | Sensitivity | Specificity |
| All Samples | N5302 N3314 N3307 N4411 N7616 HaptogI N7310 N9311 N1511 N2307 | 69.6% | 84.4% |
| Combined | N2502 N6224 | | |
| Samples Separated | N5302 N3314 N3307 N4411 N7616 HaptogI N7310 N9311 N1511 N2307 | 82.3% | 82.7% |
| N5302 = 0 + N5302 > 0 | N2502 N6224 | | |

Table 26: Enhanced sensitivity obtained by applying multivariate linear discriminant biostatistics to the blood serum concentrations of the listed protein biomarkers. The first approach employs comparing Alzheimer's disease patients and age-matched control using the listed biomarkers without sorting the compared groups. The second approach employs the separation of both Alzheimer's disease patients and age-matched control subjects into two categories based on the detection or lack of detection of Apolipoprotein E4 protein N5302 in their blood serum. A multivariate biostatistical analysis is applied to each of the 2 groups, employing all the biomarkers listed (N3314, N3317, N4411; N7616, HP-1 total [N1514+N2401+N2407+N3409], N7310, N9311, N1511, N2307, N2502, and N6224), followed by summing the separate results of the 2 multivariate biostatistical analysis of the sorted categories. As shown, this second approach provides substantial improvement in diagnostic capability over the first, non-sorted approach. These results are illustrated graphically in FIG. 33.

TABLE 27

| Mechanism | | | Amyotrophic lateral | |
|---|---|---|---|---|
| of Neuronal | Alzheimer's disease | | sclerosis (ALS)^{§} | |
| Degeneration | Apo E4 > 0 | Apo E4 = 0 | Familial | Sporadic |
| Neuronal Oxidative | Primary | Inhibited | Primary | Inhibited |
| Stress and | | | | |
| Apoptosis | | | | |
| Autoimmune/ | Secondary | Primary | Secondary | Primary |
| Innate | | | | |
| Inflammation | | | | |

^{§}From references 17, 18.

Table 27: Observed similarity in the mechanism of neuronal degeneration in Alzheimer's disease and Amyotrophic lateral sclerosis patients, drawn from the identities, functions and observed differences in blood serum concentration of the listed biomarkers.

TABLE 28^{¥}

| | Summary multivariate | | | Summary multivariate | | |
|---|---|---|---|---|---|---|
| | statistics using | | | statistics using | | |
| | 34 biomarkers | | | 24 biomarkers | | |
| Statistical | AD | PD | ALS | AD | PD | |
| Test | (n = 22) | (n = 29) | (n = 136) | (n = 44) | (n = 24) | Normal |
| Linear | 91% | 79% | 89% | 86% | 92% | 94% |

^{¥}From reference 19

Table 28^{v}: Multivariate linear discriminant analysis as indicated by percent sensitivity of classification of each disease in mixture of population, using 34 and step disc-selected 24 serum biomarkers.

TABLE 29
The examples illustrate how the invention:

```
Provides a relational perspective from the patients to functional, pre-
clinical, and clinical studies of genomic and proteomic biomarkers
Enables differential diagnostic and disease specific mechanism
discrimination between
    Similar diseases, e.g. AD vs. ALS vs. PD; AD vs. AD-Like vs.
    Normal
    Sporadic and familial disease subcategories, e.g. Apo E4 (+) AD vs.
    Apo E4 (−) AD; and sALS vs. fALS
    Disease mechanisms, e.g. oxidative stress, apoptosis, and
    autoimmune inflammatory mechanisms of neuronal degeneration.
Provides the type of information that can be employed in the monitoring
of patients for:
    Potential drug response
    Disease severity and progression
    Potential new drug targets
Will ultimately lead to personalized medicine
```

### REFERENCES

1. Petricoin E F, et al. Lancet 359, 572-577 (2002)
2. Kuerer, H M, et al. Cancer 95:2276-2282 (2002)
3. Utermann G, et al. American J. Human Genet. 32, 339-347 (1980)
4. Corder, E. H. et al. Science 261, 921-923 (1993).
5. Greenwood P M, et al. Neuropsychology, 19(2) 199-211 (2005).
6. Poirier J, J. Psychiatry Neurosci. 21, 128-134 (1996).
7. Margolis J, et al. Nature. 1969 221, 1056-1057 (1969).
8. Orrick L R, et al. Proc. Nat. Acad. Sci. USA 70, 1316-1320 (1973).
9. Goldknopf I L, et al. J. Biol. Chem. 250, 7182-7187 (1975).
10. Goldknopf I L, et al. Biochem. Biophys. Res. Commun. 65, 951-960 (1975).
11. Olson M O J, et al. J. Biol. Chem.251, 5901-5903 (1976).
12. Goldknopf I L, et al. Proc. Nat. Acad. Sci. USA 74, 864-868 (1977).
13. Goldknopf I L, et al. Proc. Nat. Acad. Sci. USA 74, 5492-5495 (1977).
14. Anderson L, et al. Proc. Nat. Acad. Sci. USA 74, 5421-5425 (1977).
15. Klose J, Human Genetic. 26, 231-243 (1975)
16. O'Farrell, P H, J. Biol. Chem. 250, 4007-4021 (1975).
17. Goldknopf I L, et al. Biochem. Biophys. Res. Commun. 342, 1034-1039 (2006).
18. Sheta E A, et al. Exp. Rev. Proteomics 3, 45-62 (2006).
19. Goldknopf I L. Exp. Rev. Mol. Diag. 7, 339-343 (2007).
20. Ekdahl C T, et al. Proc. Nat. Acad. Sci. USA 100(23) 13632-13637 (2003).
21. McGeer P L, et al. Ann. NY Acad. Sci. 1035, 104-116 (2004).
22. Licastro F, et al. Immunity & Aging 2(8) (2005).
23. Town T, et al. J. Neuroinflam. 2(24), (2005).
24. Giunta S. Immunity & Ageing 3(12), 2006.
25. Omigui S. Immunity & Ageing 4(1), (2007).
26. Shaftel S S, et al. J. Clin. Invest. 117(6), 1595-1604 (2007).
27. Lemere C A. J. Clin. Invest. 117(6), 1483-1485 (2007).
28. Forsberg P O, et al. J. Biol. Chem. 265(5), 2941-2946 (1990).
29. Eckdahl K N, et al. J. Immunol. 154(12), 6502-6510 (1995).
30. Nilsson-Eckdahl K, et al. Biochem J. 328(2), 625-633 (1997).
31. Finehout E J, et al. Dis. Markers 21(2), 93-101 (2005).
32. Alexianu M E, et al. Neurology 57, 1282-1289 (2001).
33. He Y, et al. Exp. Neurol. 176, 322-327 (2002).
34. Ballock D A, et al. Hippocampus 14(5), 649-661 (2004).
35. Sidor M M, et al. J. Neuroimmunology. 165(1-2), 104-113 (2005).
36. Daveau M, et al. Biochem J. 292, 485-492 (1993).
37. Mukhopadhyay D, et al. J. Biol. Chem. 279(12), 11119-11128 (2004).
38. Harrington C R, et al. Amer. J. Pathol. 145(6), 1472-1484 (1994).
39. Coon K D, et al. J. Clin. Psychiatry 68(4), 613-618 (2007).
40. Morishima-Kawashima M, et al. Amer. J. Pathol. 157, 2093-2099 (2000).
41. Wisniewski T, et al. Amer. J. Pathol. 145, 1030-1034 (1994).
42. Strittmatter W J, et al. Proc. Nat. Acad. Sci. USA 91, 11183-11186 (1994).
43. Gee J R, et al. Int. J. Biochem. Cell Biol. 37, 1145-1150 (2005).
44. Premkumar D R D, et al. Amer. J. Pathol. 148, 2083-2095 (1996).
45. Bondi M W, et al. Neurol. 64(3), 501-508 (2005).
46. Poirier J. J. Psychiatry Neurosci. 24, 147-153 (1999).
47. Lauderback C M, et al. Brain Res. 924, 90-97 (2002).
48. Lauderback C M, et al. Biochemistry 40, 2848-2854 (2001).
49. Butterfield D A, et al. Neurobiology. Aging 23, 655-664 (2002).
50. Butterfield D A, et al. Peptides 23, 1299-1309 (2002).
51. Varadarajan S, et al. J. Struct. Biol. 130, 184-208 (2000).
52. Yatin S M, et al. Neurobiology. Aging 20, 325-330 (1999).
53. Yao Y, et al. J. Neuroinflammation 1(21), 2004.
54. Yakovlev A G, et al. NeuroRx: J. Amer. Soc. Exp. NeuroTherapeutics 1, 5-16 (2004).
55. Nakagawa T, et al. J. Cell Biol. 150, 887-894 (2000).
56. Allen J W, et al. FASEB J. 13, 1875-1882 (1999).
57. Lavie G, et al. Brain Res. 901, 195-201 (2001).
58. Callaway J K, et al. Br. J. Pharmacology. 132, 1691-1698 (2001).
59. Callaway J K, et al. Stroke 30, 2704-2712 (1999).
60. Motine K S, et al. Amer. J Pathol. 150, 437-443 (1997).
61. Cutler R G, et al. Proc. Nat. Acad. Sci. USA 101, 2070-2075 (2004).
62. Marella M, et al. J. Biol. Chem. 282(33), 24146-24156 (2007).
63. Huber V C, et al. J. Neuroinflammation 3(1), (2006).
64. Zweig M H, et al. Clin. Chem. 39, 561-577 (1993).

What is claimed is:

1. A Method of use of protein biomarkers of neurodegenerative disease comprising two or more biomarkers in a biological sample, wherein the detection and/or the concentration of a first biomarker is employed to sort between categories of neurodegenerative disease patients and categories of normal and disease controls, and the presence and/or concentration of the first biomarker and of one or more additional biomarkers are then employed within each category for screening, diagnosis, differential diagnosis and monitoring of neurodegenerative disease severity and of disease mechanisms in the patients.

2. The method of claim 1 wherein the biological sample is blood.

3. The method of claim 2 wherein the blood sample is blood serum, or blood plasma, or whole blood, or blood cells.

4. The method of 1 wherein the biological sample is Cerebrospinal Fluid, urine, or tissue.

5. The method of claim 1 wherein the neurodegenerative disease is Alzheimer's disease (AD).

6. The method of claim 1 wherein the neurodegenerative disease is Parkinson's disease (PD).

7. The method of claim 1 wherein the neurodegenerative disease is Amyotrophic Lateral Sclerosis (ALS).

8. The method of claim 1, wherein the biomarkers comprise two or more of proteins, such as an Apolipoprotein E4 protein, an Apolipoprotein E3 protein, an Apolipoprotein A-IV protein, a Transthyretin protein, A Complement Factor H protein, A Complement Factor Hs protein, a Complement Factor Bb protein, a Complement Factor I protein, a Complement C3c1 protein, a Complement C3c2a protein, a Complement C3dg protein, a Haptoglobin HP-1 protein, an Immunoglobulin Light Chain Protein, and/or an Inter-alpha Trypsin Inhibitor protein in a blood serum sample, for distinguishing between different categories of patients with Alzheimer's disease, and for screening, diagnosis, differential diagnosis and monitoring of Alzheimer's disease severity and disease mechanisms in the patients.

9. The method of claim 8, for screening, diagnosis, differential diagnosis, and determining and monitoring of disease severity and mechanisms of Alzheimer's disease in patients, comprising:
obtaining a biological sample from a test subject;
determining whether or not a quantity of the first biomarker can be detected; and if so determining the quantity of that first biomarker in the biological sample; and
determining the quantities of one or more of the other biomarkers in the biological sample; and
determining the quantities of one or more additional biomarkers, in biological samples from normal control individuals, from patients with Alzheimer's disease, with Parkinson's disease, and with Alzheimer's disease-like (AD-like) and/or mixed disorders, wherein the detection of a quantity and/or the quantity of the first biomarker in the test subject biological sample is indicative of a particular form or variation of Alzheimer's disease or a normal condition with a potential to develop that particular form or variation of Alzheimer's disease, and the quantities of the first biomarker and of one or more additional biomarkers, in the biological sample of the test subject outside the range of that particular form or variation of Alzheimer's disease values are indicative of the absence of that form of Alzheimer's disease and the presence of a normal condition, or another neurological disorder, such as Parkinson's disease, or an Alzheimer's disease-like and/or mixed disorder, and wherein a lack of detection of a quantity and/or the quantity of the first biomarker in the test subject biological sample is indicative of another particular form or variation of Alzheimer's disease or a normal condition with a potential to develop that other particular form or variation of Alzheimer's disease, and the quantities of the first biomarker and of one or more other biomarkers in the biological sample of the test subject within the ranges of that other particular other form or variation of Alzheimer's disease values is indicative of the presence of that other particular form or variation of Alzheimer's disease, and the quantity of one or more other biomarkers, in the biological sample of the test subject outside the range of that other form of Alzheimer's disease values are indicative of the absence of that other particular form or variation of Alzheimer's disease and the presence of a normal condition, or another neurological disorder, such as Parkinson's disease, or an Alzheimer's disease-like or mixed disorder.

10. The Method of claim 9 wherein the Alzheimer's disease-like or mixed disorder is any one of a number of neurological disorders with symptoms similar to Alzheimer's disease, such as:
Frontotemporal dementia (FTD); Lewy body dementia (LBD); Corticalbasal Ganglionic degeneration, Alcohol related dementia; Semantic dementia; Vascular (Multi-infarct) dementia; Stroke (CVA); Post-irradiation Encephalopathy and Seizures; Alzheimer's disease combined with Vascular (Multi-Infarct) dementia; Alzheimer's disease combined with Lewy body dementia; Parkinson's disease combined with Lewy body dementia; Alzheimer's and Parkinson's disease combined with Lewy body dementia; Frontotemporal dementia combined with Chronic Inflammatory Demyelinating Polyneuropathy; and Thalamic CVA combined with HX of Lung CA, or Parkinson's disease or any of a number of other diseases where the disease causes symptoms similar to Alzheimer's disease.

11. The method of claim 1 wherein the detection and/or determination of quantities of biomarkers are performed by gel electrophoresis

12. The method of claim 11 wherein the detection and/or determination of quantities of biomarkers are performed by quantitative 2D gel electrophoresis.

13. The method of claim 1 wherein the detection and/or determination of quantities of biomarkers are performed by any form of immunoassay.

14. The method of claim 13 wherein the immunoassay is an ELISA assay.

15. The method of claim 14 wherein the immunoassay is an array of ELISA assays.

16. The method of claim 1 wherein the detection and/or determination of quantities of biomarkers are performed by Mass Spectrometry.

17. The method of claim 1 wherein the detection and/or determination of quantities of biomarkers are performed by chromatography
